(19) United States
(12) Patent Application Publication
(10) Pub. No.: US 2025/0313804 A1
(43) Pub. Date: Oct. 9, 2025

(54) METHODS FOR EXPANDING NATURAL KILLER CELLS (NK CELLS)

(71) Applicant: XNK THERAPEUTICS AB, Huddinge (SE)

(72) Inventor: Jan Per-Henrik HOLMQVIST, Huddinge (SE)

(21) Appl. No.: 18/705,176

(22) PCT Filed: Oct. 24, 2022
(86) PCT No.: PCT/EP2022/079547

§ 371 Date: Apr. 26, 2024

(30) Foreign Application Priority Data
Oct. 26, 2021 (GB) 2115389.5

(51) Int. Cl. C12N 5/0783; A61K 35/17; A61K 40/15

(52) U.S. Cl. CPC C12N 5/0646; A61K 35/17; A61K 40/15; C12N 2501/2302; C12N 2501/515

(57) ABSTRACT

The present invention relates to methods for simultaneously expanding and activating a population of natural killer (NK) cells, compositions obtained from those methods, and uses thereof.

The present invention relates to methods for expanding and activating a population of natural killer (NK) cells, compositions obtained from those methods, and uses thereof.

Natural killer (NK) cells are increasingly implicated in the therapeutic benefit of established cancer therapies. They represent a promising therapeutic option for patients with various types of malignant disease (Passweg et al., 2006; Rubnitz et al., 2010). One of the most experimented approaches has been adoptive transfer of autologous or allogeneic cytotoxic effectors with tumor cell killing potential to trigger a graft-vs-tumour (GvT) effect. Among the various effector populations that have a potential anti-tumor effect, NK and NK-like T cells stand out with their high cytotoxic capacity (Sutlu T and Alici E., 2009).

Despite notable progress and development of different strategies to optimize the therapeutic value of NK cells, NK cell-based immunotherapy in general had to deal with several challenges, which have limited its efficacy.

NK cells are normally present only in low numbers in peripheral blood mononuclear cells (PBMCs). An option to increase the number and function of donor-derived NK cells is to expand and activate the cells ex vivo before transfer to the patient. Therefore, NK cell expansion protocols are required that not only efficiently induce NK cell proliferation and activate NK cell function but also fulfil regulatory requirements for safety. Furthermore, compounds used during NK cell expansion must not be harmful to the patient. Different protocols have recently been established that claim to meet these requirements and allow the production of NK cells of clinical grade quality. However, the next challenge is the transfer of these protocols to clinical scale in a manageable, Good Manufacturing Practice-compliant (cGMP) way.

Methods have been developed involving GMP-compliant components that allow expansion of polyclonal NK cells in cell culture flasks using PBMCs from healthy donors (Carlens S. et al., 2001 and U.S. Ser. No. 10/242,788), as well as patients with B-cell chronic lymphocytic leukaemia (Guven H. et al, 2003), and multiple myeloma (MM) (Alici E. et al., 2008). These cells have been shown to exert specific cytotoxic activity against fresh human tumour cells in vitro and in experimental models of human tumours (Guimaraes F. et al., 2006) which opens up the possibility to be evaluated in clinical settings. However, the conventional flask-based culture is labour-intensive and cumbersome, thus limiting the cell number that can be handled practically. Previously disclosed protocols (e.g. Miller J S. et al, 1994; Pierson B A. et al, 1996; Luhrn J. et al, 2002; HG Klingemann and J Martinson Cytotherapy. 2004; 6(1):15-22) directed to effector cell preparation also include steps such as NK precursor or CD56 separation prior to culture and the use of feeder cells or cGMP-incompatible components. These disadvantages render previous protocols suboptimal and unfeasible to support large clinical studies.

A multitude of necessary hands-on steps complicate the routine use of these scaled-up manual approaches as a standard therapy. For example, expansion of NK cells in cell culture flasks has the inherent risk of exposure to external agents and contamination. Although this risk is minimized in GMP laboratory environments, the use of closed automated systems is preferred as long as it supplies sufficient amounts of cells. In contrast, partial automation of cell cultivation by use of a bioreactor has shown improved NK cell production in large scale, but because such systems require a high cell number to initiate the culture, a manual pre-cultivation step is often necessary until enough cells are generated to start the automated process. Alternatively, large volumes such as apheresis products or whole-unit peripheral blood are needed (Sutlu et al., Cytotherapy, 2010; 12:1044-1055). For example, in relation to the expansion of T cells, a 2 L perfusion Xuri Cellbag bioreactor culture requires at least 3-5×10⁷ mononuclear cells.

However, since NK cells in humans can be found circulating in the peripheral blood at low levels (representing 2-18% of lymphocytes), obtaining large numbers of NK cells to use as a starting material from fragile patients, such as cancer patients, is problematic.

Against this background the inventors have devised a simplified and semi-automated GMP-compatible method for the large-scale expansion and activation of NK cells. In the method presented here, a population of cells (including NK cells) with a concentration of less than 0.5×10⁶ cells/ml of culture media, was sufficient to initiate the current process, and so a manual pre-cultivation step is unnecessary. This is beneficial as it could reduce the blood sampling volume from fragile patients. It is also surprising that NK cell expansion could be achieved from these low starting concentrations as reduced cell-cell contacts are known to affect cell proliferation and activation.

The present method resulted in a highly pure population of NK cells in less than 25 days. The method does not require feeder cells which is advantageous since the presence of residual feeder cells in the final product is of major concern for clinical applications. Surprisingly, CD38 expression in NK cells was also reduced to a lower level in the expanded population relative to the initial population, which may be advantageous in the treatment of cancer such as multiple myeloma (MM).

On average, the method can generate 13.7×10⁹ total cells, comprising 8-9×10⁹ NK cells within a closed culture system within approximately 25 days. Approximately 5×10⁶ to 1×10⁸ NK cells/kg are needed to dose a patient suffering from a cancer such as multiple myeloma, and so this would fall within the range of NK cells typically used in NK cell immunotherapy.

In an aspect, the invention provides a method for expanding a population of natural killer (NK) cells, the method comprising:
i. providing an initial population of cells comprising NK cells;
ii. introducing the initial population of cells into a closed culture system at a concentration of less than 0.5×10⁶ cells/ml of cell culture medium; and
iii. expanding the initial population of cells in the closed culture system, under perfusion conditions effective to produce an expanded population of NK cells, preferably wherein the number of NK cells in the expanded population has increased 50-fold relative to the number of NK cells in the initial population.

By “natural killer cells (NK cells)” we include the meaning of large granular lymphocytes (LGL) that differentiate from the common lymphoid progenitor, like B and T lymphocytes. NK cells comprise 5% to 20% of human peripheral blood lymphocytes and are derived from CD34⁺ hematopoietic progenitor cells. NK cells are known to differentiate and mature in the bone marrow where they then enter the circulation. NK cells differ from natural killer T cells (NKTs) phenotypically, by origin and by respective effector functions; often, NKT cell activity promotes NK cell activity by secreting interferon-γ (IFNγ). In contrast to NKT cells, NK cells do not express the T cell marker CD3 but they usually express the surface markers CD56 in humans. Preferably, the NK cells have the phenotype CD3⁻CD56⁺.

By the terms “expanding”, “expansion” or “proliferation” we include cell growth and multiplication of cell numbers. Expansion or proliferation, as used herein, relate to increased numbers of cells, in particular NK cells, occurring during the culturing process as described herein. The term “culturing process” as used herein refers to the culturing and expansion of NK cells, wherein the starting day (starting point) of the culturing process, i.e. when the initial population of cells is seeded into the closed system, is defined as day 0. The culturing process may last as long as desired by the operator and can be performed as long as the cell culture medium has conditions which allow the cells to survive and/or grow and/or proliferate. Alternatively, the culturing process may continue until certain “release parameters” are met. Such parameters are described below in more detail and in Table 2.

It will be appreciated that when the method of the invention comprises the addition of an NK cell activating agent, the method can be considered to be a method of expanding and activating a population of NK cells. NK cells express many receptors that can activate their cytotoxic and secretory functions. Activated NK cells execute effector functions through different mechanisms. NK cells mediate direct cytotoxicity via the exocytosis pathway with release of cytotoxic granules containing granzymes and perforin, resulting in lysis of the target cell. Additionally, NK cells induce apoptosis of target cells by expression of death receptor ligands, such as Fas ligand or tumour necrosis factor-related apoptosis-inducing ligand (TRAIL). NK cells also modulate the immune response by their ability to produce pro-inflammatory cytokines, most notably Interferon-γ (IFN-γ), Tumour necrosis factor (TNF), granulocyte/monocyte colony-stimulating factor (GM-CSF), and/or chemokines (CCL1, CCL2, CCL3, CCL4, CCL5, and/or CXCL8) which facilitate the activation of T cells and other innate immune cells.

In the context of the present invention by the terms “activation” of NK cells and “activating” NK cells we include the meaning of providing NK cells with an activating signal and/or agent so that the NK cells gain cytotoxic activity. Activating signals and/or agents are discussed herein. Assays to determine whether an NK cell is activated are known in the art and are described herein. The natural cytotoxicity receptors (NCR) (NKp30, NKp44 and NKp46) are among the earliest identified NK cell-activating receptors.

By “initial population of cells” we include the starting material for the expansion which must comprise a proportion of NK cells. In an embodiment, the initial population of cells comprises or consists of NK cells. In an embodiment, the initial population of cells also comprises T cells. Due to variability between individual patient samples, the inventors have observed between 5-20% NK cells in the PBMCs used as the exemplified initial population of cells. In an embodiment, the initial population of cells is obtained from a healthy subject. In an alternative embodiment, the initial population of cells is obtained from a subject in need of cell therapy, such as a subject with cancer.

In an embodiment, step (ii) comprises introducing the initial population of cells into a closed culture system at a concentration of less than 0.5×10⁶ cells/ml, such as less 0.4×10⁶ cells/ml, 0.3×10⁶ cells/ml, 0.25×10⁶ cells/ml, 0.2×10⁶ cells/ml, 0.175×10⁶ cells/ml, 0.15×10⁶ cells/ml, 0.125×10⁶ cells/ml, 0.10×10⁶ cells/ml, 0.075×10⁶ cells/ml, or 0.05×10⁶ cells/ml of cell culture medium. In an embodiment, step (ii) comprises introducing the initial population of cells into a closed culture system at a concentration that does not exceed 0.5×10⁶ cells/ml. In an embodiment, step (ii) comprises introducing the initial population of cells into a closed culture system at a concentration that does not exceed 0.4×10⁶ cells/ml. In an embodiment, step (ii) comprises introducing the initial population of cells into a closed culture system at a concentration that does not exceed 0.3×10⁶ cells/ml. In an embodiment, step (ii) comprises introducing the initial population of cells into a closed culture system at a concentration that does not exceed 0.25×10⁶ cells/ml. In an embodiment, step (ii) comprises introducing the initial population of cells directly into a closed culture system at a concentration between 0.1×10⁶ cells/ml to 0.5×10⁶ cells/ml. In an embodiment, an initial population comprising 0.125×10⁶ cells/ml, or less, is introduced directly into the closed culture system.

As shown in the accompanying Examples, the inventors successfully expanded and activated NK cells even when starting from low concentrations of PBMCs, and without the need to grow the cells in flasks first. This was surprising for several reasons. Firstly, reduced cell-cell contacts were expected to negatively affect cell proliferation and activation. Secondly, the exemplified method comprises activating T cells in order to produce cytokines, mainly IL-2, but it was unknown whether the activation would be as effective at lower cell densities.

Accordingly, step (ii) comprises culture initiation and activation and comprises seeding the initial population of cells directly in a closed culture system comprising a suitable cell culture medium for NK cell expansion. In an embodiment, the initial population of cells was introduced into the closed culture system such that the cell concentration was less than 0.5×10⁶ cells/ml.

By “cell culture medium” we include the meaning of liquids providing the chemical conditions which are required for NK cell maintenance. Examples of chemical conditions which may support NK cell expansion are known in the art and include but are not limited to solutions, buffers, serum, serum components, nutrients, vitamins, cytokines, and other growth factors.

Suitable media for NK cells are: X-Vivo™ serum-free media (BioWhittaker, Verviers, Belgium), AIM VR serum-free medium (Thermo Fisher Scientific, Grand Island, NY, USA), CellGro Stem Cell Growth medium (SCGM) (Cell Genix, Freiburg, Germany), or complete Roswell Park Memorial Institute 1640 (BioWhittaker, Verviers, Belgium). Other media include Glycostem Basal Growth Medium (GBGM®) (Clear Cell Technologies, Beernem, Belgium), which is free of animal-derived components. Media suitable for use to cultivate NK cells as known in the art also includes, TexMACS (Miltenyi), BINKIT NK Cell Initial Medium (Cosmo Bio USA), DMEM/F12, NK Cell Culture Medium (Upcyte technologies).

Steps (ii) and (iii) of the methods described herein can be performed in a closed culture system. By “closed culture system” or “closed system” we include the meaning of a culture vessel and accessory components which reduces the risk of cell culture contamination while performing culturing processes such as the introduction of new material and performing cell culturing steps such as proliferation, differentiation, activation, and/or separation of cells. The vessels and components are utilized without breach of the integrity of the system, permit fluid transfer in and/or out while maintaining asepsis, and are connectable to other closed systems without loss of integrity. Such a system allows to operate under GMP or GMP-like conditions (“sterile”) resulting in cell compositions which are clinically applicable. The Xuri™ Cell Expansion System W25 (Cytivia) is used herein as an exemplary closed system.

By “perfusion conditions” we include the meaning of conditions allowing culture media exchange that do not involve manual handling. During a perfusion culture, waste media containing metabolic products like lactate is withdrawn from the culture and fresh complete medium is added. Accordingly, perfusion conditions allow the continuous feeding of the cells with fresh media and removal of spent media while retaining cells in culture. Typically, during perfusion, there are different ways to keep the cells in culture while removing spent media. One way is to keep the cells in the system by using capillary fibres or membranes to which the cells bind. Another method is to utilize a “lily pad” floating filter that retains the cells in the culture vessel while allowing the media to be removed. For example, a porous polyethylene-based perfusion filter that floats on the medium can be used to retain cells in the culture vessel. Another method relies on gravity settling of cell aggregates which allows for removal of spent media without the use of filtration systems. By continuously removing spent media and replacing it with new media, nutrient levels are maintained for optimal growing conditions and cell waste product is removed to avoid toxicity.

Preferably, the perfusion conditions are conditions of continuous perfusion. By “continuous perfusion” we include the meaning of a constant flow of fresh culture medium into the culture vessel (e.g. bag) while there is an equal flow of cell-free culture supernatant out of the culture vessel (e.g. bag) or as stated above “continuous “feed” of fresh medium into the closed system combined with a continuous outflow of cell free harvest”.

The terms “automated method” or “automated process” as used herein refer to any process being automated through the use of devices and/or computers and computer software which otherwise would or could be performed manually by an operator. Methods that have been automated require less human intervention. In some instances the method of the present invention is automated if at least one step of the present method is performed without any human support or intervention. Alternatively, the method of the present invention is automated if all steps of the method as disclosed herein are performed without human support or intervention. Preferentially the automated process is implemented on a closed system such as the Xuri Cell Expansion System W25.

As used herein the term “culturing” includes providing the chemical and physical conditions (e.g., temperature, gas) which are required for NK cell maintenance. Typically, the cells are incubated at between 23 and 39° C., and preferably at 37° C. Preferably the cells are cultured at a temperature of about 36-40° C., at a CO₂ concentration of about 4.7-5.1%. More preferably, at a temperature of 37° C., at a CO₂ concentration of 5.0%. Often, culturing the cells includes providing conditions for expansion (proliferation). Preferably, step (ii) comprises introducing the initial population of cells into a closed culture system at a concentration of less than 0.5×10⁶ cells/ml in a cell culture medium comprising one or more NK cell activating agent. Examples of chemical conditions which may support NK cell expansion and activation are discussed in detail herein and include those known in the art but are not limited to the use of buffers, serum, nutrients, vitamins, antibiotics, cytokines and/or growth factors which are regularly provided in the cell culture medium suited for NK cell expansion. In an embodiment, the cell culture medium comprises at least one cytokine. GMP-grade cytokines (recombinant human IL-2, IL-15, IL-12, IL-18, and IL-21), antibodies (anti-CD3-OKT3), and other ancillary reagents (nicotinamide-NAM) may serve as medium supplements for NK cell expansion. In an embodiment, the cell culture medium comprises human serum. In an embodiment, the cell culture medium comprises 5% human serum.

Illustrative examples of suitable concentrations of each cytokine or total concentration of cytokines include about 25 U/mL, about 50 U/mL, about 75 U/mL, about 100 U/mL, about 125 U/mL, about 150 U/mL, about 175 U/mL, about 200 U/mL, about 250 U/mL, about 300 U/mL, about 350 U/mL, about 400 U/mL, about 450 U/mL, about 500 U/mL, about 550 U/mL, about 600 U/mL, about 650 U/mL, about 700 U/mL, about 750 U/mL, about 800 U/mL, about 950 U/mL, or about 1000 U/mL or any amount therebetween of cytokine. In an embodiment, the suitable concentrations of each cytokine or total concentration of cytokines is about 500 U/mL.

In an embodiment, the culture medium comprises a non-ionic surfactant. In an embodiment, the non-ionic surfactant is poloxamer, such as poloxamer 188. In an embodiment, the non-ionic surfactant is Pluronic®, such as Gibco® Pluronic® F-68.

In an embodiment, the method produces an expanded cell population which comprises at least 10%, 15% or 20% NK cells, such as at least 21%, 22%, 23%, 24%, 25%, 26%, 27%, 28%, 29% or 30% NK cells. In an embodiment, the method produces an expanded cell population which comprises at least 30% NK cells, such as 35%, 40%, 45%, 50%, 55%, 60%, 65%, 70%, 75% or 80% NK cells. The method is preferably performed until at least about 20%, such as about 30, 40, 50, 60, 70, or 80% of the expanded cell population comprises NK cells. In an embodiment, the expansion is performed until at least about 30% of the expanded cell population comprises NK cells. As shown in the accompanying Examples, the percentage of NK cells at harvest was between 31 and 98% with a median of 61%.

In an embodiment, during the method of the invention the number of NK cells expands 50-fold relative to the number of NK cells in the initial population. The fold expansion of NK cells is calculated as the total cell number at harvest multiplied by the NK-cell percentage at harvest divided by the total cell number at the start of manufacturing multiplied by the NK-cell percentage at the start. During the method the number of NK cells can be increased by at least 2, 3, 4, 5, 6, 7, 8, 9, 10, 15, 20, 25, 30, 35, 40, 45, 50, 60, 70, 75, 80, 90, 100, 110, 120, 125, or 130-fold relative to the initial number of NK cells present at the beginning of the expansion. During the method, the number of NK cells can be increased by at least 200, 220, 240, 260, 280, 300, 320, 340, 360, 380, 400, 420, 440, 460, 480, 500, 520, 540, 560, 580, 600, such as 650, 700, 750, 800, 850, 900, 950 or 1000-fold relative to the number of NK cells present at the beginning of the expansion. In an embodiment, the expansion is performed until the number of NK cells has expanded 50-fold relative to the number of NK cells in the initial population.

As shown in the accompanying Examples, the number of NK cells can be increased by at least 200-fold, such as 210, 220, 230, 240, 250, 260, 270, 280, 290, or 300-fold relative to the number of NK cells present in an initial population from a healthy donor, and at least 50-fold, such as 60, 70, or 80-fold in an initial population from a cancer patient.

Preferably, the method is carried out ex vivo. By “ex vivo” we include the meaning of conditions of treating or performing a procedure on a cell(s), tissue and/or organ which has been removed from a subject's body. The cell(s), tissue and/or organ may be returned to the subject's body in a method of surgery or treatment.

Preferably, the method does not comprise the use of accessory cells as feeder cells, for example antigen-presenting “feeder” cells.

In an embodiment, the method is compatible with Good Manufacturing Practice (GMP) (see EudraLex The Rules Governing Medicinal Products in the European Union Volume 4 Good Manufacturing Practice Guidelines, part IV Good Manufacturing Practice specific to Advanced Therapy Medicinal Products).

As used herein, the term “about” or “approximately” refers to an amount, level, value, number, frequency, percentage, dimension, size, quantity, weight, or length that varies by as much as 15%, 10%, 9%, 8%, 7%, 6%, 5%, 4%, 3%, 2%, or 1% from the referenced amount, level, value, number, frequency, percentage, dimension, size, quantity, weight, or length. In particular embodiments, the term “about” or “approximately” when preceding a value, means a value plus or minus 15%, 10%, 5%, or 1% of the range.

### Preferably Step (ii) Comprises:

(a) introducing the initial population of cells into a closed culture system at a concentration of less than 0.5×10⁶ cells/ml of cell culture medium comprising one or more NK cell activating agent; and
(b) culturing the cells at a concentration that does not exceed about 1×10⁶ cells/ml.

In an embodiment, step (ii) (b) comprises culturing the cells from the initial population at a concentration that does not exceed 1.0×10⁶ cells/ml, such as a concentration that does not exceed 0.9×10⁶ cells/ml, 0.8×10⁶ cells/ml, 0.7×10⁶ cells/ml, 0.6×10⁶ cells/ml, such as 0.5×10⁶ cells/ml, 0.4×10⁶ cells/ml, 0.3×10⁶ cells/ml, 0.25×10⁶ cells/ml, 0.2×10⁶ cells/ml, 0.175×10⁶ cells/ml, 0.15×10⁶ cells/ml, or a concentration that does not exceed 0.125×10⁶ cells/ml.

In a preferred embodiment, during step (ii) the culture volume is increased to the desired volume, such as the operating volume of the culture vessel used. This may be the maximal operating volume permitted in a given culture vessel, which can be determined according to the manufacturer's instructions. By “maximum operating volume” we include the meaning of the maximum volume of cell culture that a given culture vessel can maintain viable cells. For example, the Xuri CellBag 50 L has a maximal operating volume of 25 litres (L), the Xuri CellBag 10 L has a maximal operating volume of 5 L, the Xuri CellBag 2 L has a maximal operating volume of 1 L. Accordingly, it is preferred that step (iii) begins once the culture volume is increased to the maximal operating volume permitted in a given culture vessel.

In a preferred embodiment where the culture volume is increased (for example, to the maximal operating volume permitted in a given culture vessel), during the culture volume increase the cell concentration is increased to, or maintained at, a concentration of less than or equal to 0.5×10⁶ cells/ml.

It is preferred that once the culture volume has increased (for example, to the maximal operating volume permitted in a given culture vessel), the cell concentration is also permitted to increase before perfusion commences in step (iii). Preferred cell concentrations at which perfusion may commence include those provided below and may be a cell concentration of about 1×10⁶ cells/ml.

As shown in the exemplary method, the cells were seeded directly into the bioreactor at 0.25×10⁶ cells/ml and the cell concentration was maintained at or below 0.5×10⁶ cells/ml by adding cell culture media until the culture volume reached 1 L. The cell concentration was then allowed to increase to about 1×10⁶ cells/ml. The method then progressed to step (iii). The inventors surprisingly found that this favours NK cell growth because of reduced competition for nutrients and reduced accumulation of inhibitory by-products.

### Preferably Step (ii) Comprises:

(a) introducing the initial population of cells into a closed culture system at a concentration of less than 0.5×10⁶ cells/ml in a cell culture medium comprising one or more NK cell activating agent; and optionally increasing the cell culture volume to the maximum operating volume of the cell culture vessel while increasing or maintaining the cell concentration at a concentration of less than or equal to 0.5×10⁶ cells/ml; and
(b) culturing the cells at a concentration that does not exceed about 1×10⁶ cells/ml.

In an embodiment, step (ii) comprises culturing the initial population of cells for about 4-12 days, such as about, 4, 5, 6, 7, 8, 9, 10, 11, or 12 days.

Preferably, step (ii) comprises culturing the initial population of cells for about 4-10 days, optionally 5 days.

In an embodiment, step (ii) comprises culturing the initial cell population for a time sufficient to activate one or more of the NK cells and/or for a time sufficient for the cell population to reach a concentration of about 1×10⁶ cells/ml culture medium.

By “in a cell culture medium comprising one or more NK cell activating agent” we include culturing the initial population of cells with one or more NK activating agents in an activation reaction mixture in order to generate (one or more) activated NK cells. The terms “activation” and “activated NK cells” are defined herein. Methods for determining whether an NK cell is activated, or is “cytotoxic” are described herein.

By “one or more NK cell activating agent” we include one or more agents (e.g. antibodies or functional fragments thereof) which are capable of activating NK cells. Examples of NK cell activating agents include molecules which target a T-cell stimulatory, or co-stimulatory, molecule. It will be appreciated that the NK cell activating agent may directly or indirectly activate NK cells. By directly activate NK cells we include the meaning that the agent acts on the NK cell directly (e.g. binds to a molecule on the NK cell surface) to activate it, and by indirectly activate NK cell we include the meaning that the agent activates the NK cell by virtue of having an effect on one or more molecules which in turn activate the NK cell directly. For example, an anti-CD3 antibody can induce secretion of cytokines from T cells which help to activate and expand NK cells. Any combination of one or more NK cell activating agents can be used to produce activated NK cells. Other known NK cell activating agents include antibodies against CD335 (NKp46) and CD2. Preferably, the initial population of cells (such as isolated PBMCs) are activated within the closed system. The one or more activating agents can be added to the culture media of the closed culture system without exposing the initial population of cells (e.g. PBMCs) to the environment. A reaction mixture is typically formed within the chamber of the closed system to perform the activating. The reaction mixture can be formed by adding one or more NK cell activating agents to the cell culture medium. Preferably, the one or more activating agents are used in effective amounts such that activated NK cells are produced.

Various antibodies and functional fragments thereof are known in the art to activate or stimulate NK cells. In an embodiment, the NK cell activating agent is one or more of an anti-CD2, anti-CD335 anti-CD3, and/or an anti-CD28 antibody. In illustrative embodiments, anti-CD3 antibody can be added to the media. Preferably, the NK cell activating agent comprises an anti-CD3 antibody or CD3 binding agent at the following concentrations: about 0.5 ng/ml, about 0.75 ng/ml, about 1 ng/ml, about 2.5 ng/ml, about 5 ng/ml, about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, or about 200 ng/mL, or any intermediate concentration. More preferably, the NK cell activating agent comprises an anti-CD3 antibody or CD3 binding agent at 10 ng/mL.

Preferably, the NK cell activating agent is an anti-CD3 antibody.

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab. Anti-CD3 antibodies also include the UHCT1/UCHT1 clone, also known as T3 and CD3s. Other anti-CD3 antibodies include, for example, otelixizumab, teplizumab, and visilizumab. The term “OKT-3” (also referred to herein as “OKT3”) refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/ml, MACS GMP CD3 pure, Miltenyi Biotech, Inc., San Diego, CA, USA) and muromonab or variants, conservative amino acid substitutions, glycoforms, or biosimilars thereof. As shown the accompanying Examples, an anti-CD3 antibody (OKT-3) and IL-2, can be added to the media during step (ii) (a). In an embodiment, the anti-CD3 antibody is washed out (i.e. removed) from the culture medium during perfusion (i.e. step (iii)).

In an embodiment, GMP-grade monoclonal anti-CD3 antibody (OKT-3) was added at a final concentration of 10 ng/mL.

Activating the initial population comprising NK cells seeded in the closed system may be performed for at least at least 1 hour, at least 5 hours, at least 10 hours, at least 20 hours at least 24 hours, at least 2 days, at least 3 days, at least 4 days, at least 5 days, at least 6 days or any intervening period of time. Preferably, the NK cell activating agent (e.g. an anti-CD3 antibody) is present in the cell culture for 4-6 days, preferably 5 days.

Activation (also termed “induction” herein) may be performed on an initial population of cells comprising freshly isolated cells (e.g. PBMCs) or previously cryopreserved cells. In the event that cryopreserved cells are used, the cells may be thawed and induced after overnight recovery, i.e. on day 1 of culture.

During step (iii) and perfusion, cell number, viability and metabolite levels can be monitored using methods known in art or those described herein. As shown in the accompanying Examples, glucose/lactate measurements were carried out on a Biosen instrument (EKF Diagnostic), according to manufacturer's instructions.

Preferably, the perfusion conditions in step (iii) comprise a perfusion rate effective to maintain lactate levels at 35 mM or less, such as 30 mM or less.

In an embodiment, the perfusion conditions in step (iii) comprise a perfusion rate effective to maintain lactate levels at 30 mM or less, such as at 29 mM, 29 nM, 28 mM, 27 mM, 26 mM, or 25 mM or less, such as at 20 mM, 18 mM, 15 mM, 12 mM, or at 10 mM or less.

By “perfusion rate” we include the meaning of the rate at which culture medium is delivered and removed from the closed culture system. Perfusion rate is expressed as mL medium/24 h. For example, a perfusion rate of 500 ml/24 h equates to the removal and replacement of 500 ml of culture medium in a 24 h period. It will be appreciated that this can be carried out using a non-static perfusion bioreactor system, such as Xuri W25, Xuri W5 (formerly WAVE), Biostat RM, Allegro XRS, ThermoFisher High Performer Rocker.

Preferably, step (iii) is performed until the total number of cells has expanded at least 10-fold relative to the total number of cells in the initial population.

In an embodiment, the expansion is performed until the total number of cells has expanded at least about 10-fold, such as about 20, 30, 40, 50, 60, 70, 80, 90, 100, 110, 120, 130, 140, 150, 160, 170, 180, 190, 200, 210, 220, 230, 240 or at least about 250-fold relative to the total number of cells in the initial population. In an embodiment, the expansion is performed until the total number of cells has expanded at least about 20-fold relative to the total number of cells in the initial population. In an embodiment, the expansion is performed until the total number of cells has expanded at least about 30-fold relative to the total number of cells in the initial population. The total fold expansion of cells is calculated as the total cell number at harvest divided by the total cell number at the start of manufacturing.

As shown in the accompanying Examples, the bioreactor expansion cultures with a starting concentration of 0.25×10⁶ cells/mL and a “high” perfusion rate (Table 1) showed total cell expansions between 26- and 211-fold, with a median of 112-fold.

Preferably, the initial population of cells is selected from the group comprising: a population of peripheral blood mononuclear cells (PBMCs), a population of cells derived from cord blood, a population of cells derived from a cell line, a population of cells derived from primary cells, and a population of cells derived from stem cells. Preferably, the initial population of cells comprises NK cells and T cells.

NK cells can be derived from peripheral blood; umbilical cord blood; stem cells, including induced pluripotent stem cells (IPSCs) and hematopoietic stem cells; and/or NK cell lines (such as NK-92).

NK cells can be obtained from different sources: from the patient (autologous), or from the patient's human leukocyte antigen (HLA)-matched siblings (allogenic), or haploidentical family members or unrelated donors.

The terms “peripheral blood mononuclear cells” or “PBMCs” as used herein refer to any peripheral blood cell having a round nucleus. PBMCs include lymphocytes, such as T cells, B cells, NK cells, and monocytes.

Blood containing PBMCs can be collected or obtained from a subject by any suitable method known in the art. For example, blood can be collected by venipuncture (sometimes referred to as venapuncture, venipuncture or even venu puncture) or any other blood collection method by which a sample of blood and/or PBMCs is collected. PBMCs can be isolated from the peripheral blood of donors via ficoll (GE Healthcare) gradient centrifugation. The volume of blood collected may be between 50 ml and 250 ml, for example, between 75 ml and 125 ml, or between 90 ml and 120 ml, or between 95 and 110 ml. The volume of blood collected can be between 25, 30, 35, 40, 45, 50, 55, 60, 65, 70, 75, 80, 85, 90, 95, 100, 110, 120, 130, 140, 150, 175, 200, 225, 250, 275, 300, 350, 400, 450, 500 ml. Alternatively, volume of blood collected can be between 500, 600, 700, 800, or 900 ml or 1 L.

In the methods disclosed herein, large numbers of NK cells can be generated in a short amount of time, such as 25 days, from a small volume of blood.

In some embodiments, PBMCs can be obtained by apheresis as discussed below.

NK cells may be isolated away from other components of a blood sample in an enrichment step. Such enrichment can be performed, for example, by forming a buffy coat from a blood sample using known methods. The PBMCs can be enriched by collecting the buffy coat and further enriching PBMCs from other blood components using known methods. Enrichment of PBMCs from other blood components and blood cells can be performed, for example, using apheresis, and/or density gradient centrifugation using Lymphoprep™ (STEMCELL Technologies Inc). Neutrophils may be removed before NK cells are processed. An automated apheresis separator may be used which takes blood from the subject, passes the blood through an apparatus that sorts out a particular cell type (such as, for example, PBMCs), and returns the remainder back into the subject. In some embodiments, monocytes and/or macrophages can be removed from the PBMCs using methods known in the art. For example, monocytes and/or macrophages can be removed using magnetic bead activated cell sorting or by allowing the PBMCs to grow on tissue-culture treated surfaces such that the monocytes and/or macrophages adhere, and then transferring the supernatant to a new container.

In embodiment, PBMCs are used as the initial population of cells (i.e. starting material) for NK cell cultures, either directly or after a freeze-thawing cycle.

NK cells can also be generated and/or derived from multipotent progenitor cells and pluripotent stem cells (PSCs). Highly pure, functional NK cells have been generated from CD34⁺ hematopoietic stem cells isolated from umbilical cord blood (UCB). As explained above, the initial population of cells must also comprise T cells.

NK cells can also be generated and/or derived via induced PSCs (IPSCs), as is known to those skilled in the art.

In an embodiment, the initial population of cells is cryopreserved prior to step (ii). Preferably, the cryopreserved initial population of cells is thawed, prior to step (ii).

Methods to thaw cryopreserved NK cells are known in the art. For example, cryopreserved cells can be quickly thawed at 37° C. in a water bath, bead bath, or a commercial controlled thaw rate device and transferred to a container with pre-warmed media. The cells can be washed with media to remove the cryopreservation solution. The cells can be washed with phosphate buffered saline (PBS), optionally supplemented with human serum albumin (HSA), to remove the cryopreservation solution. The cells can be allowed to recover for one or more days. The cells can be used immediately after thawing.

In an embodiment, steps (ii) and (iii) comprise culturing the cell population in the presence of one or more growth factors such as IL-2 and/or IL-15. Preferably, steps (ii) and (iii) comprise culturing the cell population in the presence of Interleukin 2 (IL-2).

Preferably, steps (ii) and (iii) comprise the addition of Il-2. Type I IFN, IL-12, IL-18 and IL-15 are activators of NK cell effector function. It will also be appreciated that IL-2 promotes NK cell proliferation, cytotoxicity and, to some extent, cytokine secretion. The term “IL-2” (also referred to herein as “IL2”) refers to the T cell growth factor known as interleukin-2, and includes all forms of IL-2 including human and mammalian forms, conservative amino acid substitutions, glycoforms, biosimilars, and variants thereof e.g. IL-2 superkine. For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, NH, USA (CELLGRO GMP) or ProSpec-Tany TechnoGene Ltd., East Brunswick, NJ, USA (Cat. No. CYT-209-b) and other commercial equivalents from other suppliers.

The term “IL-15” as used herein refers to cytokine Interleukin-15 and includes all forms of IL-15 including human and mammalian forms, conservative amino acid substitutions, glycoforms, biosimilars, and variants thereof, e.g. IL-15Rα sushi. Preferably, the cytokines are exogenous. Recombinant human IL-15 is commercially available from multiple suppliers, including ProSpec-Tany TechnoGene Ltd., East Brunswick, NJ, USA (Cat. No. CYT-230-b) and ThermoFisher Scientific, Inc., Waltham, MA, USA (human IL-15 recombinant protein, Cat. No. 34-8159-82). In some embodiments, the concentration of IL-15 in the media can be between 10, 15, 20, 25, 30, 35, 40, 45, 50, 60, 70, 80, 90, 100, 125, 150, 200, 250, or 300, 400, 500, 600, 700, 800, 900, or 1,000 U/ml.

In some embodiments, the concentration of IL-2 in the media can be between 10, 15, 20, 25, 30, 35, 40, 45, 50, 60, 70, 80, 90, 100, 125, 150, 200, 250, or 300, 400, 500, 600, 700, 800, 900, or 1,000 U/ml. Preferably, IL-2 is present at 500 U/mL. Preferably, IL-2 is added throughout steps (ii) and (iii). The media can be supplemented with IL-2 multiple times during expansion such as, every 12, 24, 36, or 48 hours. Preferably, IL-2 is continuously added to the closed system.

Preferably, the culture medium in step (ii) and (iii) comprises IL-2 and human serum. More preferably, throughout the culture, the cell culture medium is supplemented with 500 IU/mL IL-2 and 5% human serum. The medium is also supplemented with an agent capable of preventing foam formation, such as a non-ionic surfactant, such as poloxamer, preferably poloxamer 188. In an embodiment, the cell culture medium is Stem Cell Growth medium (Cellgenix) with 5% human serum, 500 U/ml IL-2; and 0.1% pluronic (F-68).

In an embodiment, the perfusion conditions in step (iii) maintain the cell-specific perfusion rate (CSPR) between 0.01-2 mL/10⁶ cells/24 h, such as between 0.02 and 1.5 mL/10⁶ cells/24 h, such as 0.02, 0.05, 0.075, 0.1, 0.5, 0.75, 1.0, 1.25 and 1.5 mL/10⁶ cells/24 h.

By “cell-specific perfusion rate (CSPR)” we include the meaning of the medium exchange rate needed to sustain a given cell population in a perfusion process. Herein, CSPR is expressed as mL culture media added per million cells per 24 hours.

The inventors have surprisingly found that NK cells can be expanded when cell populations having low cell concentrations are perfused. Thus in an embodiment, the perfusion of step (iii) starts when the cell population has a concentration of 1×10⁶ to 2×10⁶ cells/ml of cell culture medium. In an embodiment, the perfusion of step (iii) starts when the cell population has a concentration of less than or equal to 1×10⁶ cells/ml of cell culture medium. Preferably, the perfusion of step (iii) starts when the cell population has a concentration of about 1×10⁶ cells/ml of cell culture medium. It is preferred if the perfusion of step (iii) starts when the cell population has a concentration less than 3×10⁶ cells/ml.

### Preferably the Perfusion Conditions Comprise:

a perfusion rate of about 0.4-0.6× maximal culture vessel operating volume per 24 hours when the cell population has a concentration of between 1×10⁶ cells/ml and 3×10⁶ cells/ml; and/or
a perfusion rate of about 0.6-0.9× maximal culture vessel operating volume per 24 hours when the cell population has a concentration of between 3×10⁶ cells/ml and 9×10⁶ cells/ml; and/or
a perfusion rate of about 0.9-1.2× maximal culture vessel operating volume per 24 hours after the cell population has reached a concentration of at least 9×10⁶ cells/ml.

By “a perfusion rate of about 0.4-0.6× maximal culture vessel operating volume per 24 hours” we include the meaning that in a 24 hour period 0.4-0.6 of the maximal volume of the culture vessel is added to the culture vessel and 0.4-0.6 of the maximal volume of the culture vessel is removed from the culture vessel. By “0.4-0.6× maximal culture vessel operating volume” we include the meaning of a volume of the culture vessel volume that is 40-60% of the maximal operating culture volume in the culture vessel. In other words, if the maximal operating culture vessel volume is 1 L, 0.4-0.6× maximal operating culture vessel volume is 400-600 mL.

The maximal operating culture volume for a given vessel can be determined by the skilled person or will be provided by the manufacturer of the culture vessel. For example, the maximal total volume in a 2 L Xuri Cellbag bioreactor should be 1000 mL, and the maximal total volume in a 10 L Xuri Cellbag bioreactor should be 5000 mL. Preferably, during continuous perfusion, the volume of spent medium removed was equal to the volume of fresh medium added in order to maintain a constant volume. Automated medium exchange and closed-system passaging methods avoid any open steps that could introduce a contaminant, ensuring that the cell product remains sterile.

Examples of perfusion conditions are provided in Table 1. Preferably the perfusion conditions comprise “high” condition as set out in Table 1, which are applicable to a culture vessel with a maximal culture vessel operating volume of 1 L. However, these volumes could be scaled depending on the culture vessel in question.

### The “Low” Perfusion Rate Detailed in Table 1 Comprises:

a perfusion rate of about 0.1-0.4 (preferably about 0.3)× maximal culture vessel operating volume per 24 hours when the cell population has a concentration of between 0.5×10⁶ cells/ml and 3×10⁶ cells/ml; and/or
a perfusion rate of about 0.4-0.6 (preferably about 0.5)× maximal culture vessel operating volume per 24 hours when the cell population has a concentration of between 3×10⁶ cells/ml and 7×10⁶ cells/ml; and/or
a perfusion rate of about 0.6-0.9 (preferably about 0.75)× maximal culture vessel operating volume per 24 hours when the cell population has a concentration of between 7×10⁶ cells/ml and 15×10⁶ cells/ml; and/or
a perfusion rate of about 0.9-1.2 (preferably about 1.0)× maximal culture vessel operating volume per 24 hours after the cell population has reached a concentration of at least 25×10⁶ cells/ml.

### The “Medium” Perfusion Rate Detailed in Table 1 Comprises:

a perfusion rate of about 0.1-0.4 (preferably about 0.3)× maximal culture vessel operating volume per 24 hours when the cell population has a concentration of between 0.5×10⁶ cells/ml and 1×10⁶ cells/ml; and/or
a perfusion rate of about 0.4-0.6 (preferably about 0.5)× maximal culture vessel operating volume per 24 hours when the cell population has a concentration of between 1×10⁶ cells/ml and 3×10⁶ cells/ml; and/or
a perfusion rate of about 0.6-0.9 (preferably about 0.75)× maximal culture vessel operating volume per 24 hours when the cell population has a concentration of between 3×10⁶ cells/ml and 9×10⁶ cells/ml; and/or
a perfusion rate of about 0.9-1.2 (preferably about 1.0)× maximal culture vessel operating volume per 24 hours after the cell population has reached a concentration of at least 20×10⁶ cells/ml.

### For Example, the “High” Perfusion Rate Detailed in Table 1 Comprises:

a perfusion rate of about 0.4-0.6, preferably about 0.5× maximal culture vessel operating volume per 24 hours when the cell population has a concentration of between 1×10⁶ cells/ml and 3×10⁶ cells/ml; and/or
a perfusion rate of about 0.6-0.9, preferably about 0.75× maximal culture vessel operating volume per 24 hours when the cell population has a concentration of between 3×10⁶ cells/ml and 9×10⁶ cells/ml; and/or
a perfusion rate of about 0.9-1.2, preferably about 1.0× maximal culture vessel operating volume per 24 hours after the cell population has reached a concentration of at least 9×10⁶ cells/ml.

In an embodiment in which the closed system is a non-static bioreactor comprising a maximum operating culture volume of 1 L, the “high” perfusion conditions as described in Table 1 comprise:
a perfusion rate of about 400-600 ml (preferably 500 ml) culture medium/24 h when the cell population has a concentration of between 1×10⁶ cells/ml and 3×10⁶ cells/ml; and/or
a perfusion rate of about 600-900 ml (preferably 750 ml) culture medium/24 h when the cell population has a concentration of between 3×10⁶ cells/ml and 9×10⁶ cells/ml; and/or
a perfusion rate of about 900-1200 ml (preferably 1000 ml) culture medium/24 h after the cell population has reached a concentration of at least 9×10⁶ cells/ml.

The skilled person would be able to adjust the conditions above for vessels of different sizes.

In an embodiment in which the closed system is a non-static bioreactor system and a 2 L culture vessel (e.g. 2-litre bioreactor bag) is used with a maximal operating volume of 1 L, the perfusion starts with 500 ml/day when the cells reach a density of 1×10⁶ cells/ml, and after 3×10⁶ cells/ml, to 750 ml/day, and after 9×10⁶ to 1000 ml/day. If the lactate level reaches over 25 mmol/l (i.e. 25 mM), the perfusion rate is increased to the next perfusion rate, as shown in Table 1. In other words, if the lactate level reaches over 25 mmol/l, perfusion is increased.

Preferably, step (iii) comprises expanding the initial cell population for at least 9 days.

The cell expansion step can be performed for a certain number of days. Expansion can be performed for 4, 5, 6, 7, 8, 9, 10, 11, 12, 13, 14, 15, 16, 17, 18, 19, 20, 21, 22, 23, 24, or 25 days. In certain embodiments, expansion is performed for between 10 and 24 days, preferably between 12 and 22 days, preferably between 14 and 20 days.

Preferably the method further comprises the step, performed after step (iii), of:
(iv) harvesting the expanded cell population.

By “harvesting the expanded cell population” we include the meaning of terminating the expansion and/or removing the expanded cells from the closed cell system. Harvesting the expanded NK cells can be performed based on an expansion criteria or release criteria. The release criteria for the expanded NK cells herein are shown in the Table 2 below.

TABLE 2
Release criteria

| Parameter | Approval limit |
|---|---|
| Viable cell number | ≥3 × (30 × 10⁶ cells per kg bodyweight) |
| Cell viability | ≥90% viable cells |
| % NK cells (CD3⁻CD56⁺) | ≥10% of the viable cell population |
| % Activated | ≥30% CD107a⁺ cells in the CD3⁻CD56⁺ |
| NK cells | population in response to K562 cells |
| Mycoplasma | Not detectable (ND) |
| Sterility | Sterile |
| Endotoxin | ≤0.5 EU/mL |
| Tumour cell contamination | Not detectable (ND) |
| Karyotyping | No clonal aberrations |

The time for expansion can be any suitable time which allows for the production of (i) a sufficient total number of cells in the expanded population; (ii) a population of expanded cells with a favourable proportion of NK cells compared to the initial population (such as at least 10%), or (iii) both (i) and (ii).

Cell harvesting can also be performed a certain number of days after the method started. In some embodiments, harvesting can be performed 7, 8, 9, 10, 11, 12, 13, 14, 15, 16, 17, 18, 19, 20, 21, 22, 23, 24, 25, 26, 27, 28, 29, or 30 days after the method commenced. In some embodiments, harvesting can be performed between 7, 8, 9, 10, 11, 12, 13, or 14 days after the method commenced, or 10, 11, 12, 13, 14, 15, 16, 17, 18, 19, 20, or 21 days after the method commenced, or 15, 16, 17, 18, 19, 20, 21, 22, 23, 24, 25, or 26 days after the method commenced.

In some embodiments, cell harvesting is performed when the NK cells have been expanded at least 2, 3, 4, 5, 6, 7, 8, 9, 10, 11, 12, 13, 14, 15, 16, 17, 18, 19, 20, or 25-fold. For example, cell harvesting may be performed when the NK cells have been expanded at least 5 fold, at least 10 fold, or at least 20 fold. This expansion is typically measured by counting the NK cells, or viable NK cells, harvested after expansion versus the total number of NK cells, or viable NK cells, that were present in the initial cell population (such as PBMCs).

Cell harvesting can also be performed when the total cells reach a specified cell density in the culture media. In some embodiments, harvesting can be performed when the cell density is between 1×10⁵, 2.5×10⁵, 5×10⁵, 1×10⁶, 2.5×10⁶, 5×10⁶, 1×10⁷, 2.5×10⁷, 5×10⁷, 1×10⁸, 2.5×10⁸, 5×10⁸, or 1×10⁹ cells/ml. On average, the method generates 10-14×10⁹ cells (total) and 3-7×10⁹ activated NK cells.

In some embodiments, cell harvesting is performed when the total cells have been expanded at least 2, 3, 4, 5, 6, 7, 8, 9, 10, 11, 12, 13, 14, 15, 16, 17, 18, 19, 20, or 25-fold, relative to the total number of cells in the initial population. For example, cell harvesting may be performed when the total cells have been expanded at least 5 fold, at least 10 fold, or at least 20 fold. Alternatively, cell harvesting is performed when the total cells have expanded at least 20, 30, 40, 50, 60, 70, 80, 90, 100, 110, 120, 130, 140, 150, 160, 170, 180, 190, 200, 210, 220, 230, 240 or at least about 250-fold relative to the total number of cells in the initial population. This expansion is typically measured by counting the total cells, or total viable cells, harvested after expansion versus the total number of cells, or viable cells, that were present in the initial cell population (such as PBMCs).

The harvested (expanded) cells can include different percentages NK cells. The harvested cells can include at least 10%, 15%, 20%, 25%, 30%, 35%, 40%, 45%, 50%, 55%, 60%, 65%, 70%, 75%, 80%, 85%, 90%, 95%, or 100% NK cells. In some embodiments, cell harvesting is performed when the expanded cell population comprises at least 10% NK cells (CD3⁻CD56⁺), such as at least, 15%, 20%, 25%, 30%, 35%, 40%, 45% or at least 50%. Preferably, cell harvesting is performed when the expanded cell population comprises at least 10% NK cells (CD3⁻CD56⁺).

In some embodiments, cell harvesting is performed when the NK cell (CD3⁻CD56⁺) population contains at least 30% activated NK cells (i.e. CD107a⁺ cells in the CD3⁻CD56⁺ population following co-culture with K562 cells), such as at least 35%, 40%, 45%, 50%, 55% or at least 60% activated NK cells in the NK cell population.

As shown in the accompanying Examples, preferably the cells are harvested the earliest at day 15, if the release criteria are met. In some embodiments, at day 10 the NK cells are already activated and show increased degranulation against K562 cells.

In some embodiments, the harvested cells produced by the methods described herein can be cryopreserved at a predetermined dose for use at a later time. Methods and reagents for cryopreserving cells are well-known in the art. Cryopreservation can include one or more washes and/or a step of concentrating the NK cells with a diluent solution, which may be a cryopreservation solution. In some embodiments, the diluent solution can be normal saline, 0.9% saline, PlasmaLyte (Baxter, Lessines, Belgium), 5% dextrose/0.45% NaCl saline solution, human serum albumin (HSA), or a combination thereof.

In some embodiments, human serum albumin (HSA) can be added to the washed and concentrated cells for improved cell viability and cell recovery after thawing. In some embodiments, the washing solution can be PBS and washed and concentrated cells can be supplemented with HSA, for example 1%, 2%, 3%, 4%, 5%, 6%, 7%, 8%, 9%, or 10% HSA. The method can also include a step of forming a cryopreservation mixture, which includes the NK cells in the diluent solution and a suitable cryopreservation solution.

The cryopreservation solution can be any suitable cryopreservation solution including, but not limited to, CryoStor10 (BioLife Solution), mixed with the diluent solution of NK cells at a ratio of 1:1 or 2:1. The cryopreservation solution can include at least 1%, 2%, 3%, 4%, 5%, 6%, 7%, 8%, 9%, or 10% DMSO. HSA can be added to a final concentration in the cryopreservation solution of between 1%, 2%, 3%, 4%, 5% 6%, 7 8%, 9 10%, 11%, 12%, 13%, 14%, or 15% HSA. The method can include a step of freezing the cryopreservation mixture. In one aspect, the cryopreservation mixture is frozen in a controlled rate freezer using a defined freeze cycle. The method can include a step of storing the cryopreservation mixture in vapor phase liquid nitrogen or liquid nitrogen. The cells can be cryopreserved in a cryopreservation solution comprising human plasma with 5% DMSO.

In certain embodiments of the methods disclosed herein, the harvested NK cells can be introduced, reintroduced, infused, or reinfused into a subject in need of NK cell therapy for a therapeutic effect. The number of NK cells to be reintroduced can be a predetermined dose, which can be a therapeutically effective dose as provided below. The predetermined dose can depend on the nature of the disease or condition being treated. The predetermined dose of harvested cells may be based on the mass of a subject, for example, cells per kilogram of the subject (cells/kg). In any of the embodiments disclosed herein, the number of NK cells to be introduced, reintroduced, or infused back into a subject can be between 1×10³, 2.5×10³, 5×10³, 1×10⁴, 2.5×10⁴, 5×10⁴, 1×10⁵, 2.5×10⁵, 5×10⁵, 1×10⁶, 2.5×10⁶, 5×10⁶, or 1×10⁷, 2×10⁷, 3×10⁷, 5×10⁷, or 1×10⁸ cells/kg. In an embodiment, 5×10⁶ to 1×10⁸ cells/kg are given to a subject.

A subject can be, for example, an animal, a mammal, and a human. In some embodiments, the subject can be healthy. In other embodiments, the subject can be suffering from a disease or condition, such as cancer. Suitable subjects include any individual, e.g., a human or non-human animal who has cancer, who has been diagnosed with cancer, who is at risk for developing cancer, who has had cancer and is at risk for recurrence of the cancer, who has been treated with an agent for the cancer and failed to respond to such treatment, or who has been treated with an agent for the cancer but relapsed after initial response to such treatment.

In an embodiment, the closed culture system is a non-static perfusion bioreactor.

By “non-static perfusion bioreactor” we include the meaning of a system in which cells, cell culture medium, chemicals and reagents are aseptically added, removed and/or manipulated without breach of integrity of the system (e.g., by opening the cap of a tube or lifting the lid off a cell culture plate or dish). Single-use or multiple-use bags and/or containers and/or are added onto or into the bioreactor system for example by sterile tube welding at the site of the vessel or bioreactor. An exemplary bioreactor system is the Xuri™ W25 Cell Expansion System (Cytiva). The platform software provides the ability to perform continuous or discontinuous perfusion or medium exchange in a closed system, with datalogging of all aspects of cell culture, including CO₂ levels, temperature, bag weight, pump speeds, and gas exchange. Optical sensors are available for continuous monitoring of dissolved oxygen and pH, with real-time controls and data storage.

In an embodiment, the bioreactor system comprises means to control evaporation and/or condensation; means to control accumulation of waste metabolites; means to control temperature; means to control the gas flow rate; and/or means to control addition of culture medium or other additives.

In an embodiment, the bioreactor system comprises a computer terminal, a bioreactor control unit, a gas outlet line, a sensor cable, a cell expansion system, a rocking platform, a bioreactor, a gas inlet port, a sensor for measuring oxygen uptake, a media waste line, a media feed line, and pump unit, a media feed reservoir, and a media waste reservoir. The pump unit, the cell expansion system and bioreactor control unit may be connected to the computer terminal which controls the rate that the pump unit operates which controls the rate of media exchange in response to oxygen uptake readings.

Preferably, the closed culture system comprises a culture vessel positioned on a platform capable of rocking. In an embodiment, the closed system is a rocking-motion bioreactor system such as Xuri W25, Xuri W5 (formerly WAVE), Biostat RM, Allegro XRS, ThermoFisher High Performer Rocker.

By “culture vessel” we include the meaning of any sterile vessel or chamber suitable for expanding NK cells. In an embodiment, the culture vessel is single use. In an embodiment, the culture vessel comprises a cell retention means, capable of retaining cells whilst allowing culture media exchange via perfusion. Preferably, the culture vessel has low gas permeability. The culture vessel may comprise an air inlet filter; air outlet filter; needleless sampling port; inoculation/harvest lines; optical dissolved oxygen (DO) sensor; optical pH sensor; perfusion filter; screw cap; and/or aseptic connectors.

The size of the culture vessel should match the medium volume for ideal fluid motion, and can be selected by the person skilled in the art. A culture vessel described herein may have a volume of from about 50 ml to about 100 litres, about 100 ml to about 100 litres, about 150 ml to about 100 litres, about 200 ml to about 100 litres, about 250 ml to about 100 litres, about 500 ml to about 100 litres, about 1 litre to about 100 litres, about 1 litre to about 75 litres, about 1 litre to about 50 litres, about 1 litre to about 25 litres, about 1 litre to about 20 litres, about 1 litre to about 15 litres, about 1 litre to about 10 litres, about 1 litre to about 5 litres, about 1 litre to about 2.5 litres, or about 1 litre to about 2 litres.

Preferably, the bioreactor system comprises a flexible cell bag. Preferably, the flexible cell bag is a sterile bag.

The size of the cell bag should match the medium volume for ideal fluid motion, and can be selected by the person skilled in the art. A cell bag described herein may have a volume of from about 50 ml to about 100 litres, about 100 ml to about 100 litres, about 150 ml to about 100 litres, about 200 ml to about 100 litres, about 250 ml to about 100 litres, about 500 ml to about 100 litres, about 1 litre to about 100 litres, about 1 litre to about 75 litres, about 1 litre to about 50 litres, about 1 litre to about 25 litres, about 1 litre to about 20 litres, about 1 litre to about 15 litres, about 1 litre to about 10 litres, about 1 litre to about 5 litres, about 1 litre to about 2.5 litres, or about 1 litre to about 2 litres.

For example, a 1 L cell bag can be used for medium volumes between 150 and 500 mL, while a 2 L cell bag can be used for medium volumes between 400 ml and 1 L. The same parameters can be applied for both the 1 and 2 L cell bags, as the bag size scales appropriately with volume depth. Cell bags are typically available at 1, 2, 10, 20 and 50 L sizes for scalable cell culture. Optionally other Cell bags and/or vessels of larger or smaller volume can be employed for the methods described herein. In an illustrative embodiment, the cell bag is a Xuri™ CellBag.

In an embodiment, the closed system comprises a rocking motion bioreactor system. For a rocking bioreactor system, two parameters are the rock angle (maximal angle as measured from a flat resting position) and the rock speed (in rocks per minute). The speed and angle of the rocking platform are variable in order to balance oxygenation and mixing of the culture media, and shear forces.

Bioreactor systems allow for different rocking rates and a variety of different rocking angles. Exemplary rocking rates include, but are not limited to, 1, 2, 3, 4, 5, 6, 7, 8, 9, 10, 11, 12, 13, 14, 15, 16, 17, 18, 19, or 20 rocks per minute (rpm). In certain embodiments, the rocking platform angle is set at 1.5°, 2°, 2.5°, 3°, 3.5°, 4°, 4.5°, 5°, 5.5°, 6°, 6.5°, 7°, 7.5°, 8°, 8.5°, or 9.0°. The rocking angle and speed needed for a given culture vessel can be determined by using combinations of rock angle and speed, monitoring dissolved oxygen, and observing the cell count and composition of the cell population.

Preferably, the closed culture system is non-static and is maintained at a rocking rate of 4-8 rocks per minute (rpm), preferably 6 rpm. This is preferably during step (iii).

Preferably the closed culture system is maintained at an angle between 4-8 degrees, preferably 6 degrees. This is preferably during step (iii).

Preferably the closed culture system is maintained at a rocking rate of 4-8 rpm and at an angle between 4-8 degrees, preferably 6 degrees. This is preferably during step (iii).

More preferably, during step (iii) the closed culture system is maintained at a rocking rate of 6 rpm and at an angle of 6 degrees.

Preferably, the NK cells in the expanded population have increased cytotoxicity relative to NK cells in the initial population of cells.

In an embodiment, the expanded population of NK cells produced during step (iii) has increased cytotoxicity relative to the initial population of cells comprising NK cells of step (i). The inventors have observed that NK cell activity is higher in the expanded population relative to the initial population. Therefore, the NK cell product produced by the described method is highly cytotoxic.

Preferably, the cytotoxicity of the NK cells is as determined by an in vitro cytotoxicity test. A skilled person can determine the cytotoxicity using available methods. One way of determining if cells exhibit an increased cytotoxicity is to use the in vitro analysis of cell mediated cytotoxicity against K562 cells using the standard 4 hour ⁵¹Cr-release assay, at an effector cell to target cell ratio of 10:1. Alternatively, a degranulation assay can be used. For example, a degranulation assay against K562 cells, followed by measuring the percentage of degranulated cells in each lymphocyte subpopulation. Both of these assays are described in Sutlu et al., Cytotherapy, 2010; 12:1044-1055 and WO 2010/110734 (see “3. Evaluation of cell mediated cytotoxicity”). Other in vitro cytotoxicity tests are known in the art.

As shown in the accompanying Examples, the inventors have observed that the expanded cell populations from initial populations of cells with concentrations of 0.25×10⁶/ml contained 10.5%, 37%, and 53.4%, NKp44-positive NK cells. The fraction of cells that responded with degranulation during incubation with the cell line K562 was expression level of the activation marker generally correlates with functional response, both measures of activation can differ.

In an embodiment, the proportion of NK cells expressing surface CD38 in the expanded population is less than 70%.

In an embodiment, the proportion of NK cells expressing surface CD38 in the expanded population may be less than 70%, such as less than 60%, 50%, 40%, 30%, 20%, or less than 10%.

CD38 is a type II 45 kDa glycoprotein usually present in the cellular surface membrane. CD38 has multifaceted roles since it possesses properties of an activation marker, an adhesion molecule interacting with endothelial CD31 and ecto-enzymatic activity. CD38 is also an intracellular signaling protein. CD38 is an established immunotherapeutic target in multiple myeloma and under investigation as a target antigen in acute myeloid leukaemia (AML). CD38 expression on NK cells and its further induction during certain ex vivo NK cell expansion methods represents a problem to the development of anti-CD38 therapies when combined with NK cell therapy. Some groups have used CRISPR/Cas9 to knockout CD38 in primary NK cells (Kararoudi et al. Blood 2020). It will be appreciated that such approaches will knockout intracellular and extracellular CD38. The inventors have surprisingly found that the fractions of CD38-positive cells before and after Expansions 6, 7 and 9 were 95.2% and 53.6%; 86.8% and 21.6%; and 93.3% and 25%, respectively. The reduction in surface CD38 expression in the expanded NK cells using the claimed methods is advantageous because genetic manipulation, for example by CRISPR/Cas9, is not required and so the cells have no risk of genetic off-target effects and also would not be classified a genetically modified organisms (GMO). In other words, the inventors observed a down regulation of surface CD38 that is independent of genetic engineering. In an embodiment, the expanded cell population comprising NK cells has not undergone genetic engineering.

CD38 expression can be measured by any method known in the art such as FACS and western blot.

Preferably, the proportion of NK cells in the expanded population that express surface CD38 is less than the proportion of cells in the initial population that express surface CD38.

By “the proportion of NK cells in the expanded population that express surface CD38 is less than the proportion of cells in the initial population that express surface CD38” we include the meaning of that the proportion of NK cells expressing surface CD38 in the expanded population is less than the proportion of NK cells expressing surface CD38 in the initial population.

In another aspect, the invention provides a population comprising activated natural killer (NK) cells with the phenotype CD3⁻CD56⁺ obtainable or obtained by a method described herein.

In another aspect, the invention provides a population comprising activated natural killer (NK) cells with the phenotype CD3⁻CD56⁺, wherein less than 70% of the NK cells express surface CD38, optionally which are obtainable or obtained by a method described herein.

In another aspect, the invention provides a pharmaceutical composition comprising a population of activated NK cells as defined herein and a pharmaceutically acceptable, diluent, carrier, or excipient.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyl oleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavouring agents, dyes, fluid and nutrient replenishers, such like materials and combinations thereof, as would be known to one of ordinary skill in the art. The pH and exact concentration of the various components in a pharmaceutical composition are adjusted according to well-known parameters.

Liquid pharmaceutical compositions, whether in solution, suspension, or other similar form, may comprise one or more of the following: DMSO, sterile diluents such as water for injection, saline solution (preferably physiological saline), ringer's solution, isotonic sodium chloride, fixed oils (such as synthetic mono- or diglycerides which may serve as a solvent or suspending medium), polyethylene glycols, glycerol, propylene glycol or other solvents; antibacterial agents such as benzyl alcohol or methyl paraben; antioxidants, such as ascorbic acid or sodium bisulfite; chelating agents, such as ethylenediaminetetraacetic acid; buffers such as acetate, citrate or phosphate; and agents for adjusting tonicity, such as sodium chloride or glucose.

In some embodiments, cells are formulated in a unit dosage injectable form, such as a solution, suspension, or emulsion. Pharmaceutical formulations suitable for injection of cells typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof. The skilled artisan can readily determine the amount of cells and optional additives, vehicles, and/or carrier in compositions to be administered in methods of the invention.

In another aspect, the invention provides a population of activated NK cells, or the pharmaceutical composition as described herein, for use in medicine.

In another aspect, the invention provides a population of NK cells as described herein, or a pharmaceutical composition as described herein, for use in a method of adoptive cell therapy.

In another aspect, the invention provides a method for performing adoptive cell therapy comprising administering a population of NK cells as described herein, or a pharmaceutical composition as described herein to a patient in need thereof.

In another aspect, the invention provides use of a population of NK cells as described herein, or a pharmaceutical composition as described herein, in the manufacture of a medicament for adoptive cell therapy.

By “adoptive cell therapy” we include a type of immunotherapy in which NK are given to a patient to help the body fight diseases, such as cancer. Adoptive cell therapy can also be termed adoptive cell transfer, cellular adoptive immunotherapy, and NK-cell transfer therapy.

The term “autologous” refers to any material derived from the same individual to which it is later to be re-introduced. For example, cell therapy may comprise collection PBMCs from a donor, e.g., a patient, which are then expanded, and then administered back to the same donor, e.g., patient.

The term “allogeneic” refers to any material derived from one individual which is then introduced to another individual of the same species, e.g., allogeneic NK cell transplantation.

In an embodiment, the patient receives a therapeutically effective amount of NK cells. By a “therapeutically effective amount” or “therapeutically effective dosage,” we include an amount of the NK cells that are produced by the present methods and that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of the NK cells to promote disease regression can be evaluated using a variety of methods known to the skilled practitioner, such as in human subjects during clinical trials, in animal model systems predictive of efficacy in humans, or by assaying the activity of the agent in in vitro assays.

Compositions can be administered in dosages and by techniques well known to those skilled in the medical and veterinary arts taking into consideration such factors as the age, sex, weight, and condition of the particular patient, and the formulation that will be administered (e.g., solid vs. liquid).

It is to be appreciated that a single dose may be delivered all at once, fractionally, or continuously over a period of time. The entire dose also may be delivered to a single location or spread fractionally over several locations.

Cells may be administered in an initial dose, and thereafter maintained by further administration. Cells may be administered by one method initially, and thereafter administered by the same method or one or more different methods. The levels can be maintained by the ongoing administration of the cells. Suitable regimens for initial administration and further doses or for sequential administrations may all be the same or may be variable. Appropriate regimens can be ascertained by the skilled artisan, from this disclosure, the documents cited herein, and the knowledge in the art. The dose, frequency, and duration of treatment will depend on many factors, including the nature of the disease, the subject, and other therapies that may be co-administered.

A variety of other therapeutic agents may be used in combination with the compositions described herein.

In another aspect, the invention provides a population of NK cells as described herein, or a pharmaceutical composition as defined herein, for use in the treatment and/or prevention of cancer and/or a viral infection in a patient.

In another aspect, the invention provides use of a population of NK cells as defined herein, or a pharmaceutical composition as defined herein, in the manufacture of medicament for treating and/or preventing cancer and/or a viral infection in a patient.

In another aspect, the invention provides a method of treating and/or preventing cancer and/or a viral infection in a patient comprising administering a population of NK cells as defined herein, or a pharmaceutical composition as defined herein to the patient.

As used herein, “treatment” or “treating” includes any beneficial or desired effect on the symptoms or condition of a disease or pathological state, and may even include a minimal reduction in one or more measurable markers of the disease or condition being treated (e.g., cancer). Treatment may optionally include amelioration or complete alleviation of one or more symptoms of the disease or condition, or delay in the progression of the disease or condition. “treatment” does not necessarily indicate complete eradication or cure of the disease or condition or symptoms associated therewith.

As used herein, “prevent” and similar words such as “prevent”, “preventing”, and the like refer to methods for preventing, inhibiting, or reducing the likelihood of occurrence or recurrence of a disease or condition (e.g., cancer). It also refers to delaying the onset or recurrence of a disease or condition, or delaying the onset or recurrence of symptoms of a disease or condition. As used herein, “prevention” and similar words also include reducing the intensity, impact, symptoms, and/or burden of a disease or condition prior to its onset or recurrence.

A “patient” as used herein includes any mammal who is afflicted with a disease such as cancer (e.g., a lymphoma or a myeloma). The mammal may be any domestic or farm animal. Preferably, the mammal is a rat, mouse, guinea pig, cat, dog, horse, or a primate. Most preferably, the mammal is human. The terms “subject” and “patient” are used interchangeably herein. The term “donor subject” refers to herein a subject whose cells are being obtained for expansion. The donor subject can be a cancer patient that is to be treated with a population of cells generated by the methods described herein (i.e., an autologous donor), or can be an individual who donates a sample that, upon generation of the population of cells generated by the methods described herein, will be used to treat a different individual or cancer patient (i.e., an allogeneic donor). Those subjects who receive the cells that were prepared by the present methods can be referred to as “recipient subject.”

“Administering” refers to the physical introduction of an agent to a subject, using any of the various methods and delivery systems known to those skilled in the art. Exemplary routes of administration for the cell product prepared by the methods disclosed herein include intravenous, intramuscular, subcutaneous, intraperitoneal, spinal, or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Preferably, the cell product is administered via infusion into the antecubital vein of the left or right arm. Administering can also be performed, for example, once, a plurality of times, and/or over one or more extended periods.

A “cancer” refers to a broad group of various diseases characterized by the uncontrolled growth of abnormal cells in the body. Unregulated cell division and growth results in the formation of malignant tumours that invade neighbouring tissues and can also metastasize to distant parts of the body through the lymphatic system or bloodstream. A “cancer” or “cancer tissue” can include a tumour at various stages.

In a preferred embodiment, the cancer is a haematological cancer. By “haematological cancer” we include types of cancer affecting blood, bone marrow and lymph nodes, such as those selected from the group comprising or consisting of: myeloma, lymphoma, leukaemia, and chronic myeloproliferative diseases.

An article of manufacture or a kit comprising expanded NK cells is also provided herein. The article of manufacture or kit can further comprise a package insert comprising instructions for using the immune cells to treat or delay progression of cancer in an individual or to enhance immune function of an individual having cancer. Suitable containers include, for example, bottles, vials, bags, and syringes. The container may be formed from a variety of materials such as glass, plastic (such as polyvinyl chloride or polyolefin), or metal alloy (such as stainless steel or hastelloy). The container may hold the formulation and the label on, or associated with, the container may indicate directions for use. The article of manufacture or kit may further include other materials desirable from a commercial and user standpoint, including other buffers, diluents, filters, needles, syringes, and package inserts with instructions for use. The article of manufacture further may include one or more of another agent (e.g., a chemotherapeutic agent). Suitable containers for the one or more agent include, for example, bottles, vials, bags and syringes.

In another aspect, the invention provides method, a population of NK cells, a population of NK cells for use, use of a population of NK cells, pharmaceutical composition, substantially as described herein with reference to the accompanying claims, drawings, and examples.

The present invention will now be described with reference to the following non-limiting Figures and Examples.

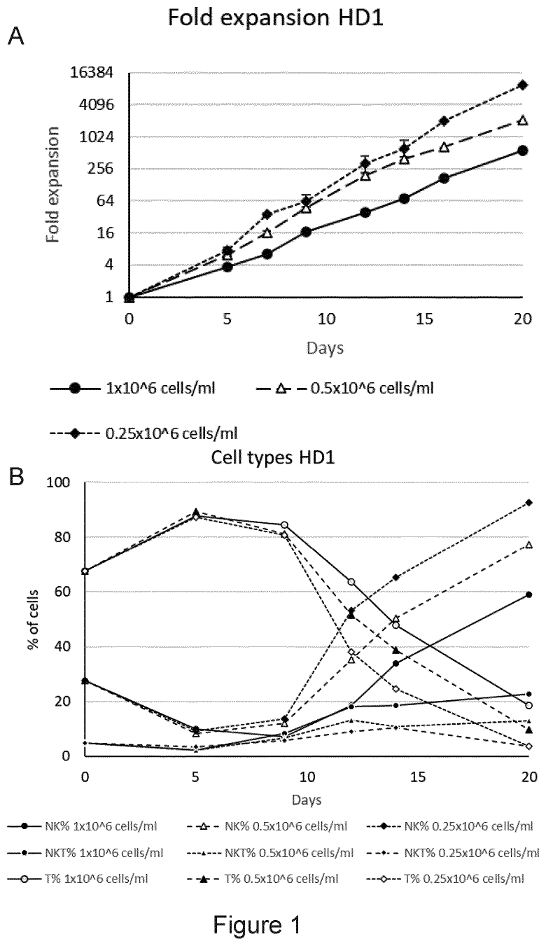

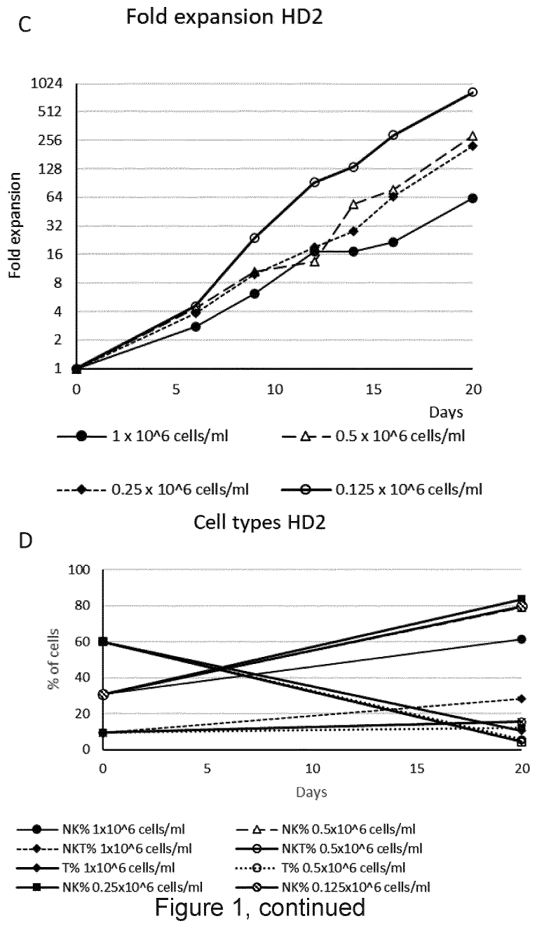

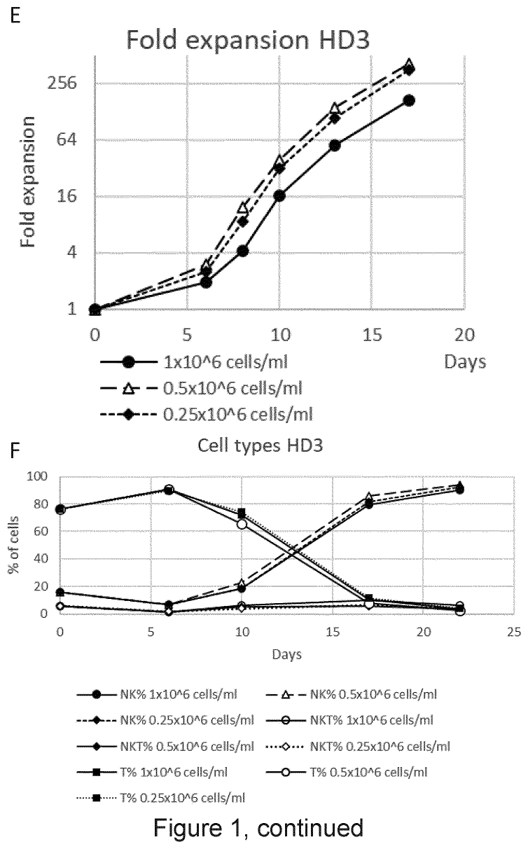

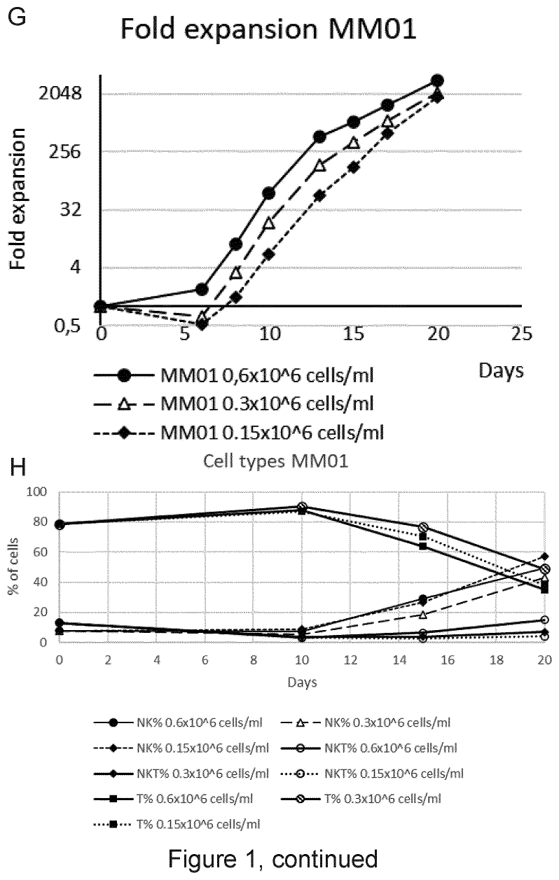

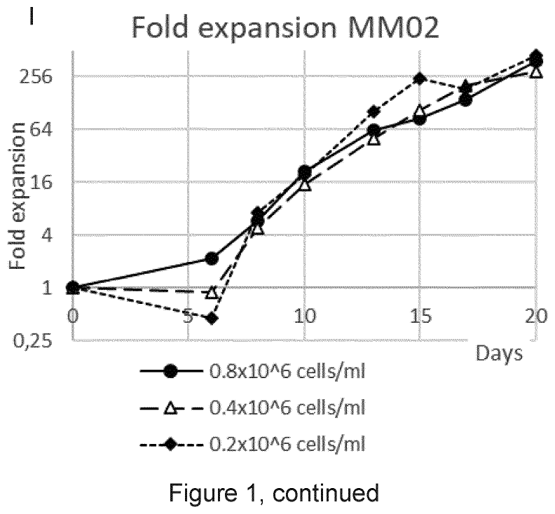

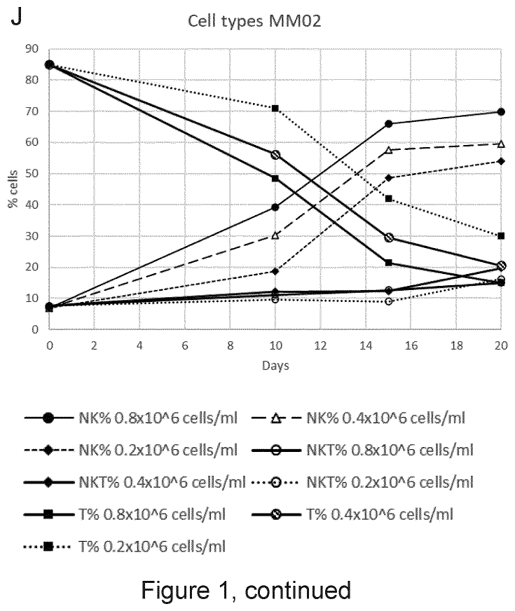

FIG. 1. Total cell expansion at different starting concentrations in three small-scale cultures of healthy donor PBMC (A, C, E) and two of MM patient PBMC (G, I). (B, D, F, H J) Lymphocyte composition in the respective cultures, as % of CD45⁺, CD19⁻ and CD14⁻ populations. NK cells are defined as CD3⁻CD56⁺, T cells as CD3⁺CD56⁻ and NKT as CD3⁺CD56⁺. Values are average and standard deviation of duplicate cultures.

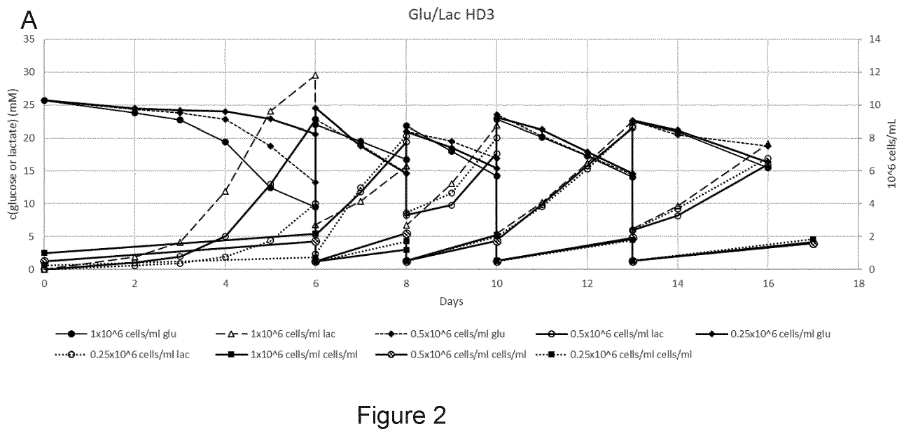

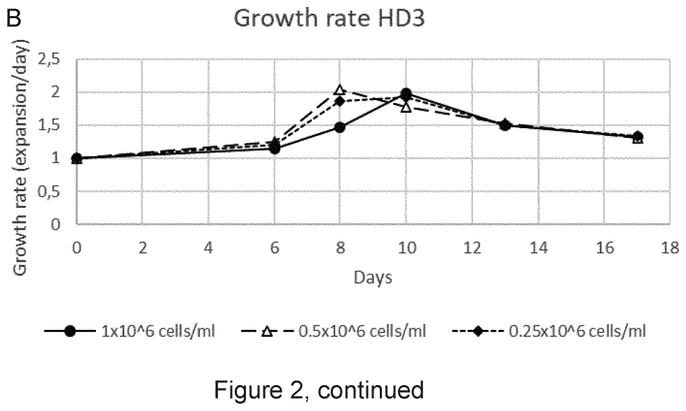

FIG. 2. (A) Glucose and lactate levels (mM) in cultures with different starting concentrations of HD6 and (B) growth rate of the cell culture. The growth rate is calculated as expansion per 24 h.

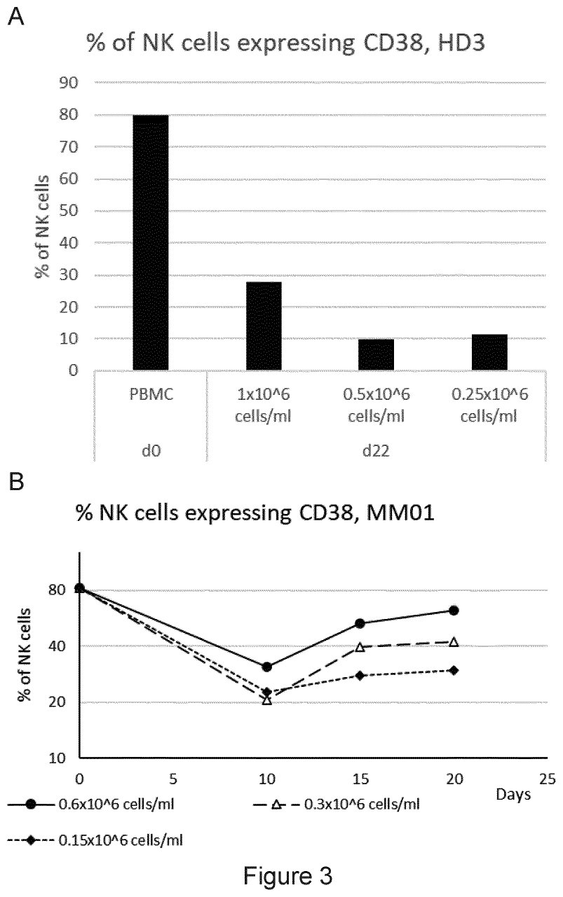

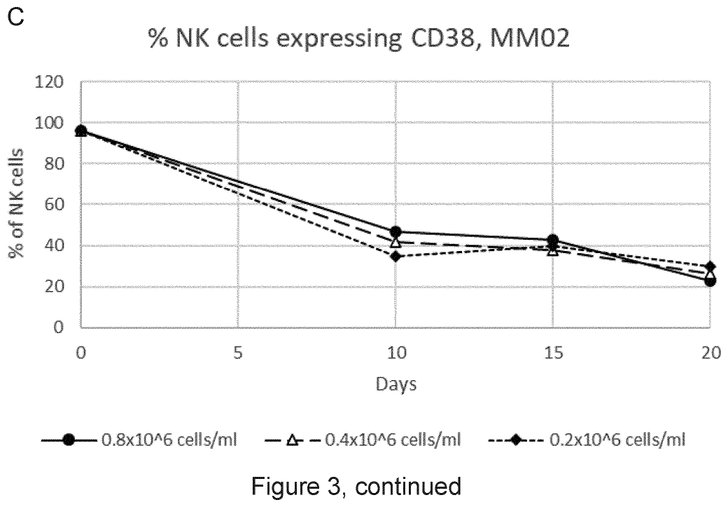

FIG. 3. CD38 expression on NK cells. (A) NK cell CD38 expression at harvest compared to the expression at culture start for HD6. (B, C) NK cell CD38 expression at different time points during culture of cells from myeloma patient MM01 (B) and MM02 (C)

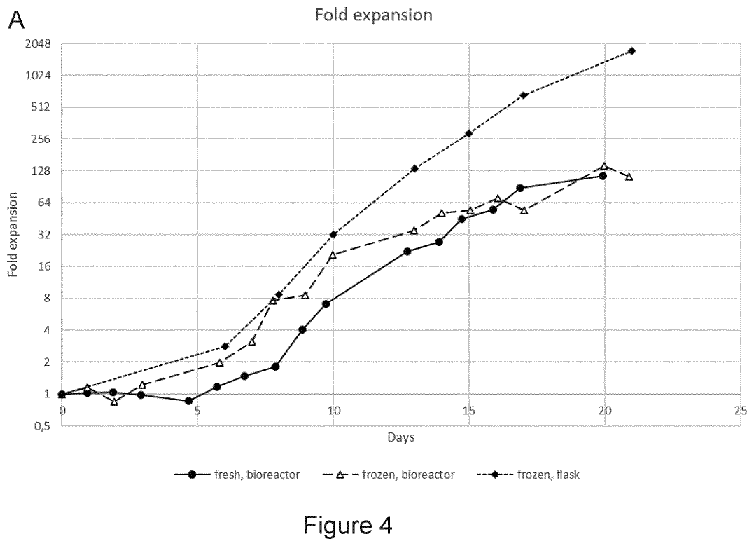

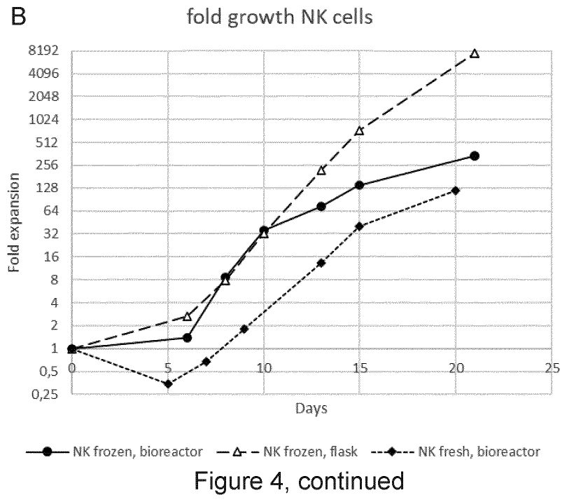

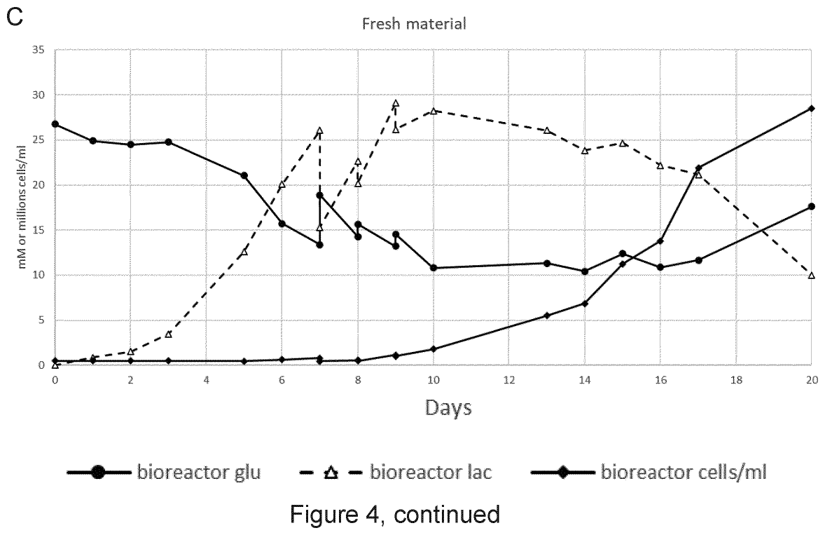

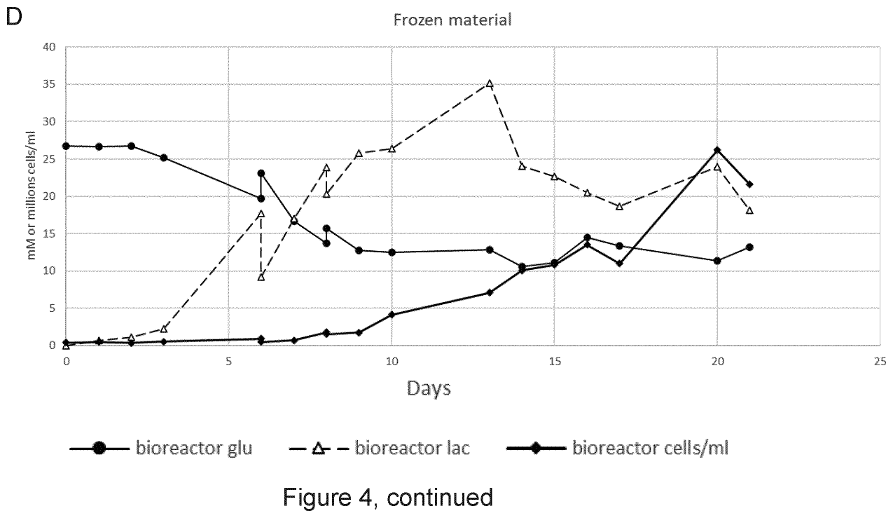

FIG. 4. Comparison of bioreactor expansions 1 (fresh) and 2 (frozen). The perfusion was ‘low’ according to Table 1. Fold expansion of total cells (A), fold expansion of NK cells (B), and glucose and lactate levels in relation to cell concentration (C, fresh material; D, frozen material).

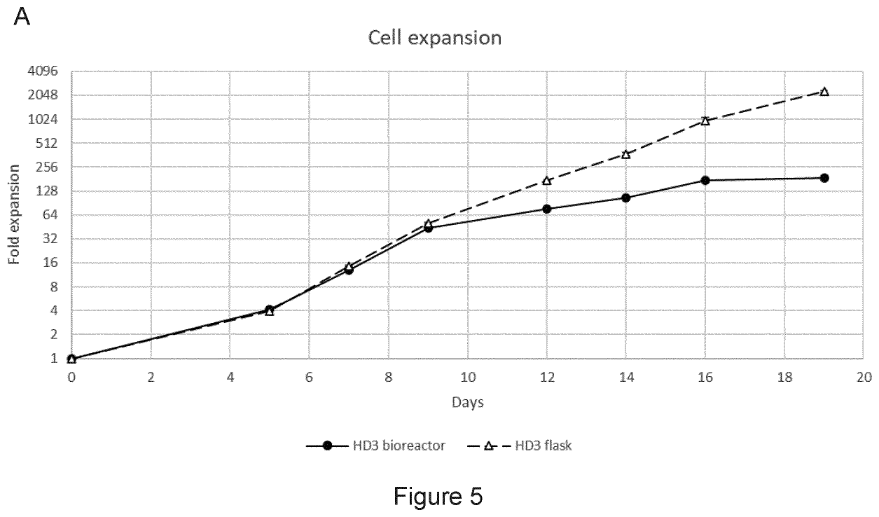

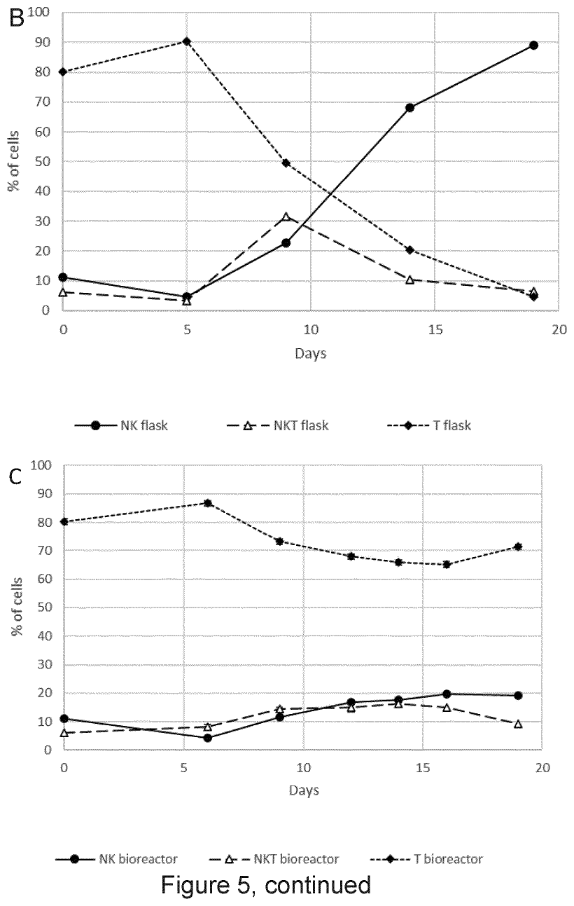

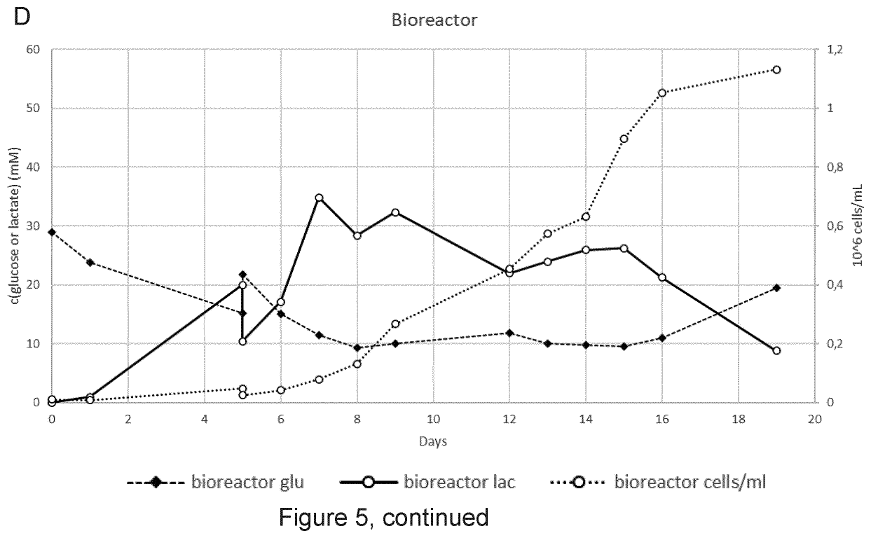

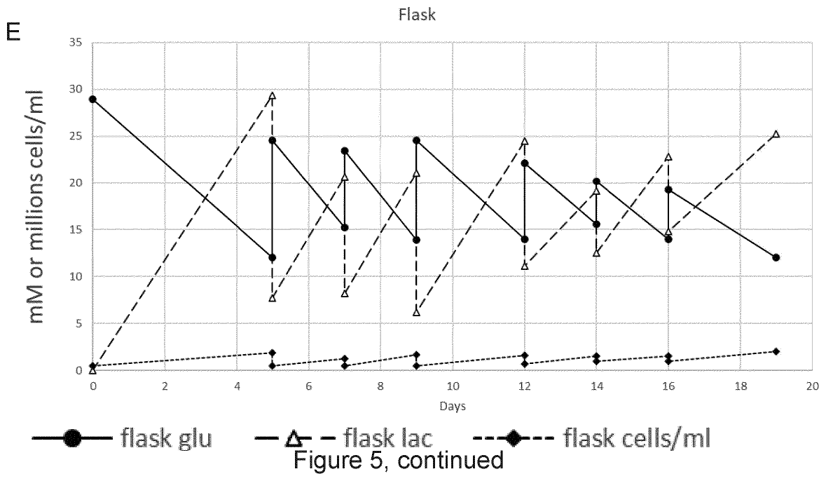

FIG. 5. Bioreactor expansion 3 in comparison to expansion in culture flasks. The perfusion was ‘low’ according to Table 1. Fold expansion of total cells (A), percentages of NK cells, T cells, and NKT cells in flasks (B) and bioreactor (C), and glucose and lactate levels in relation to cell concentration in the bioreactor (D) and flask culture (E). NK cells are defined as CD3⁻CD56⁺, T cells as CD3⁺CD56⁻ and NKT as CD3⁺CD56⁺.

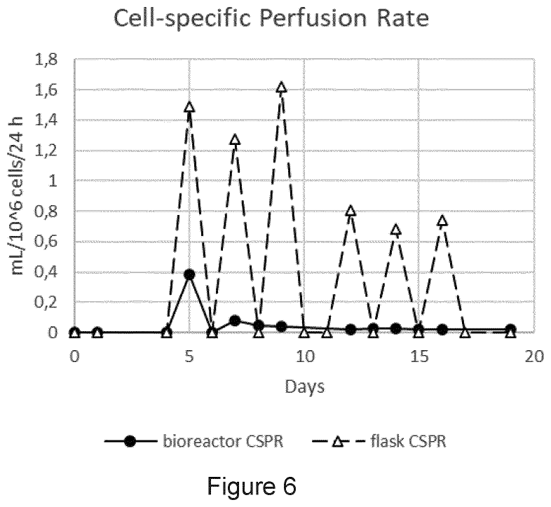

FIG. 6. Cell Specific Perfusion Rate (CSPR) in expansion 3 calculated as ml fresh media added per million cells per 24 h.

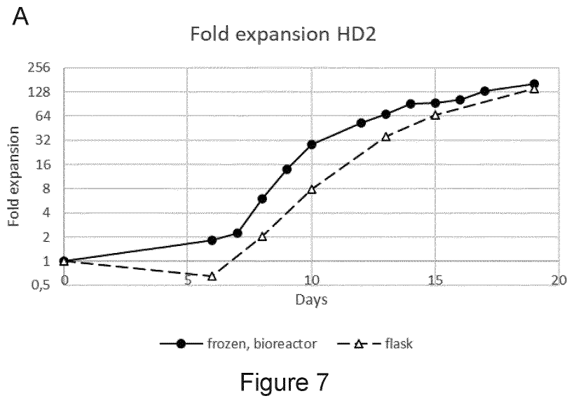

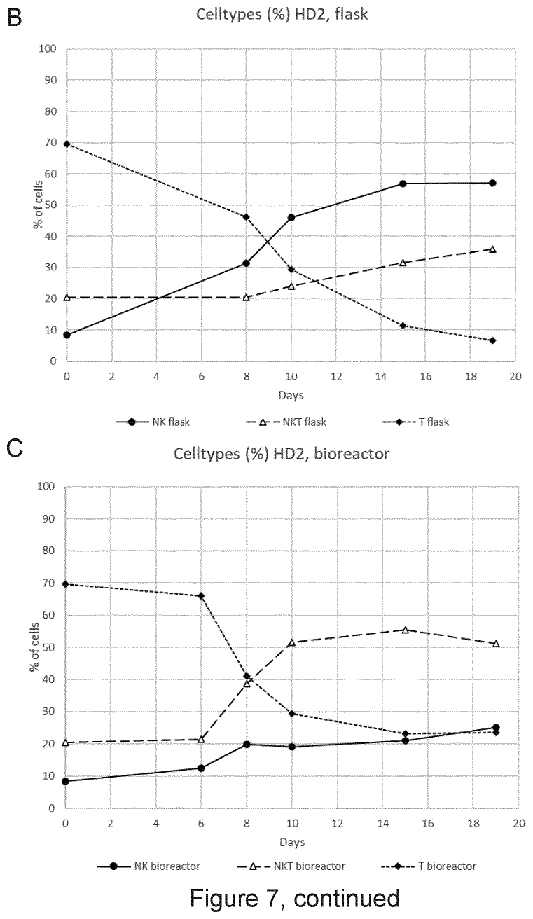

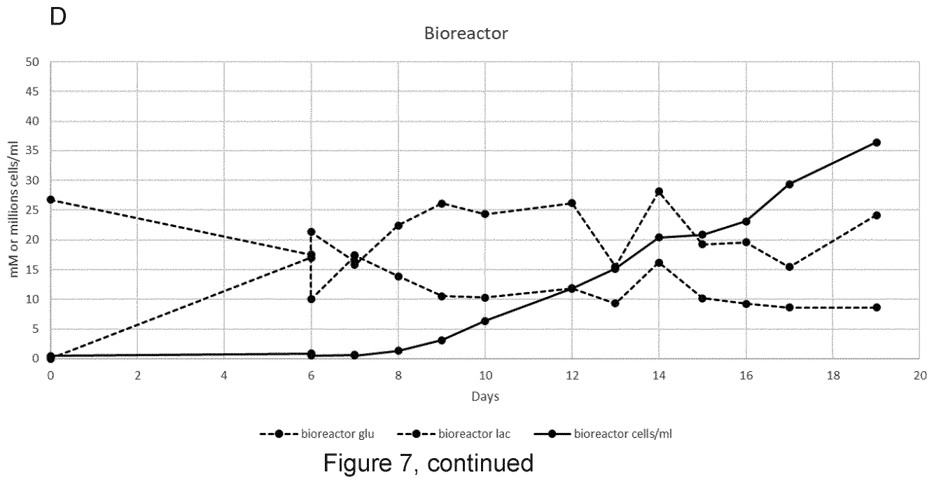

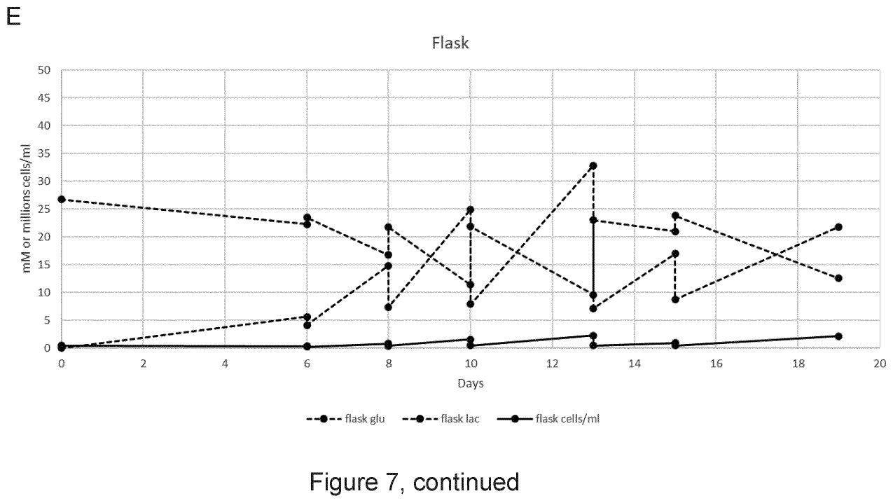

FIG. 7. Bioreactor expansion 4 in comparison to expansion in culture flasks. The perfusion regime was changed to ‘medium’ according to Table 1. Fold expansion of total cells (A), percentages of NK cells, T cells, and NKT cells in flasks (B) and bioreactor (C), and glucose and lactate levels in relation to cell concentration in the bioreactor (D) and the flask culture (E).

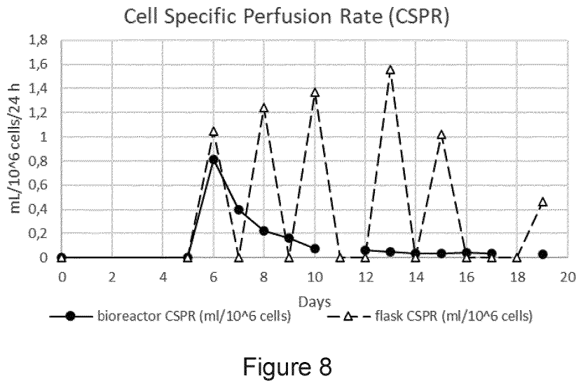

FIG. 8. Cell specific perfusion rate for expansion 4 calculated as ml fresh media added per million cells per 24 h.

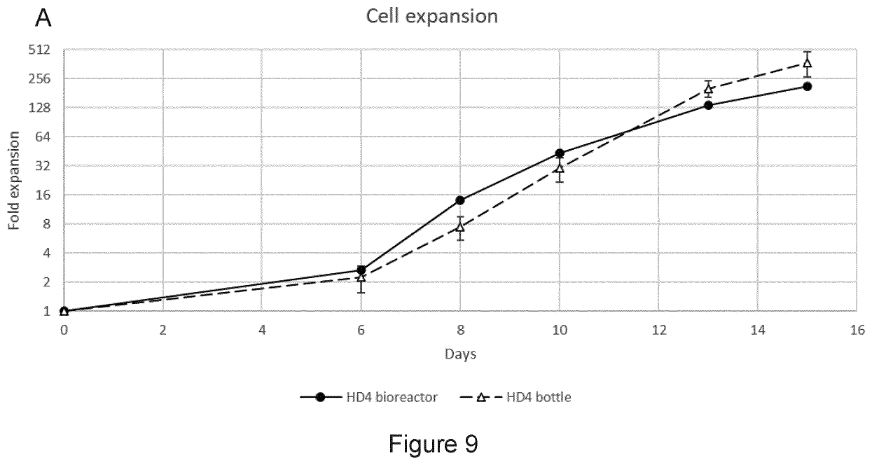

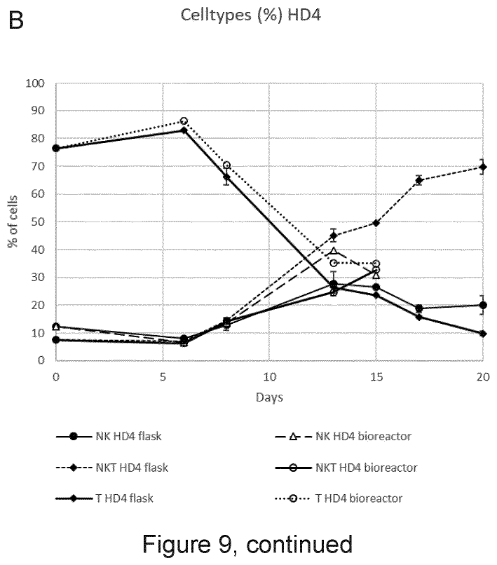

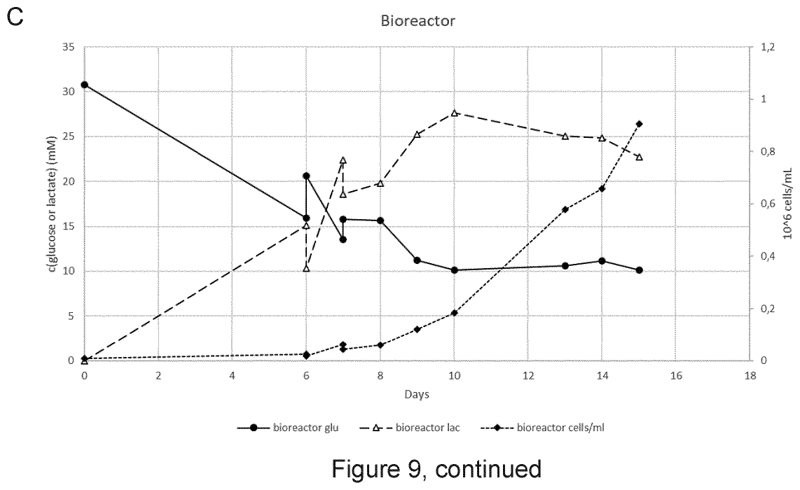

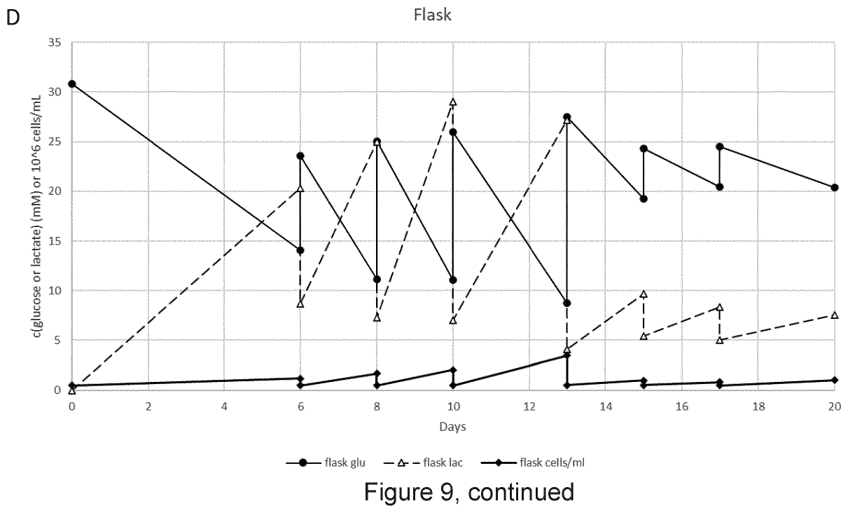

FIG. 9. Bioreactor expansion 5 with ‘high’ perfusion according to Table 1 in comparison to expansion in culture flasks. The starting concentration was 0.25×10⁶ cells/mL. Fold expansion of total cells (A), percentages of NK cells, T cells, and NKT cells (B), and glucose and lactate levels (mM) in relation to cell concentration (10⁶ cells/mL) in bioreactor (C) and flask culture (D).

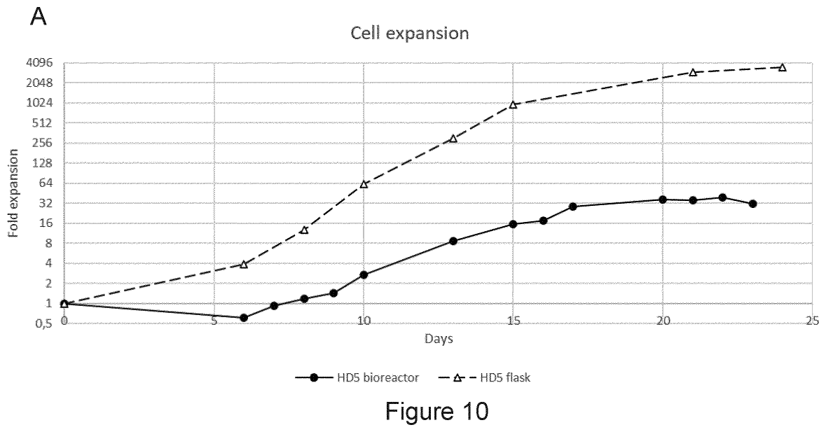

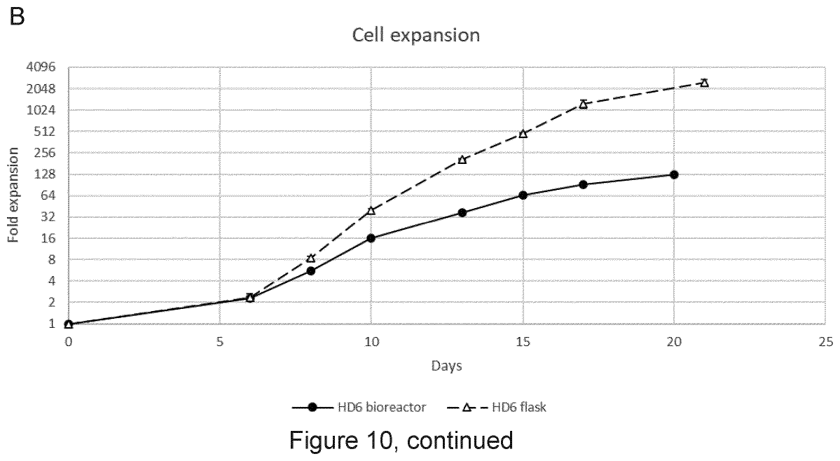

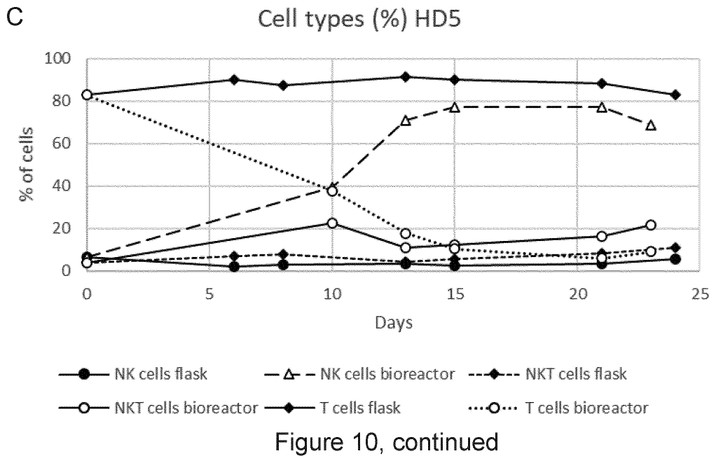

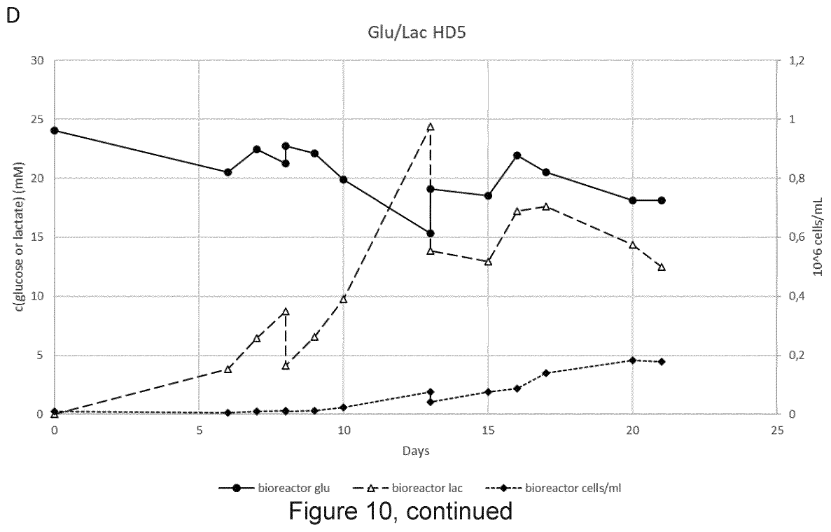

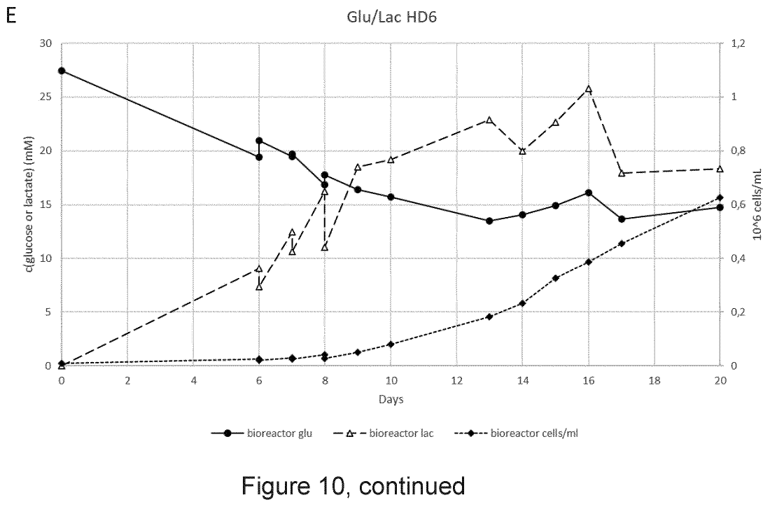

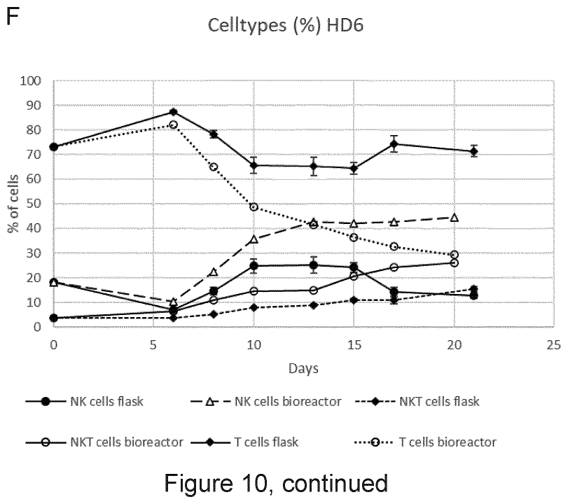

FIG. 10. Bioreactor expansions 6 and 7 in comparison to the respective flask cultures. The starting concentration was 0.25×10⁶ cells/mL and perfusion ‘high’ according to Table 1. Fold expansion of total cells in expansion 6 (A) and 7 (B), glucose and lactate levels (mM) in relation to cell concentration (10⁶ cells/mL) (D and E)), and percentages of NK cells, T cells, and NKT cells (C and F).

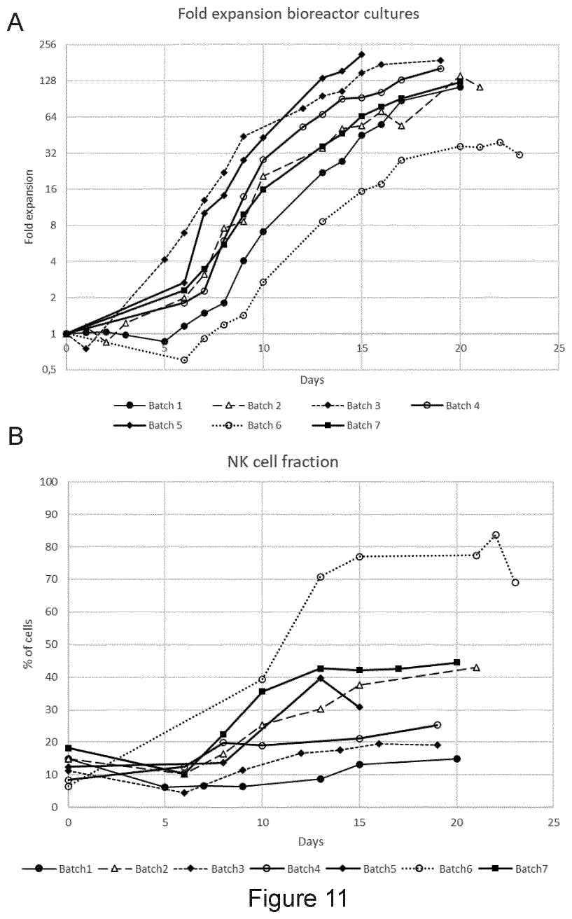

FIG. 11. Comparison of total cell expansion and percentage of NK cells in all seven bioreactor expansion cultures with healthy donor cells.

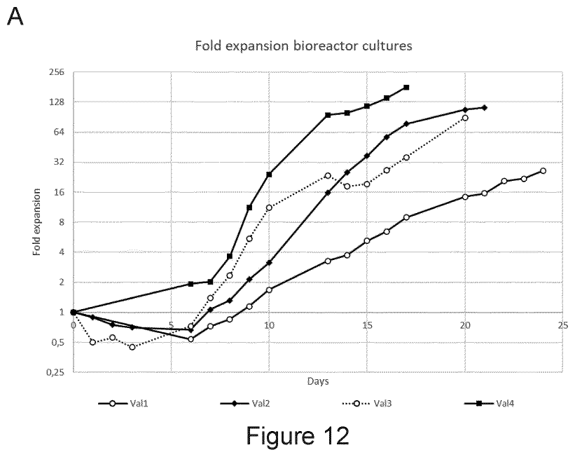

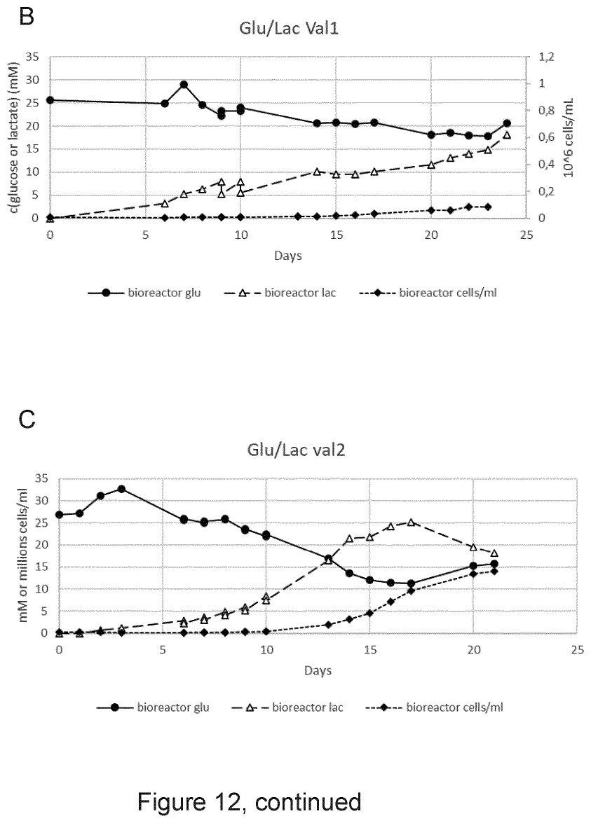

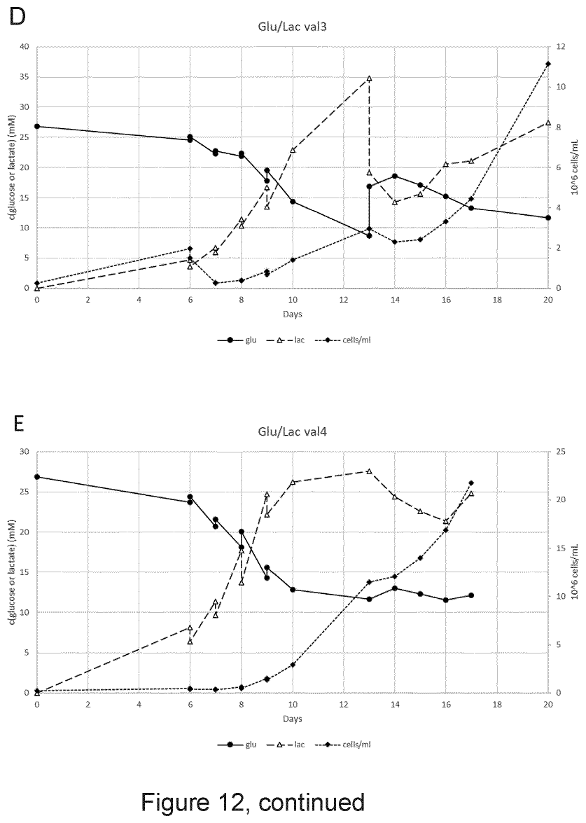

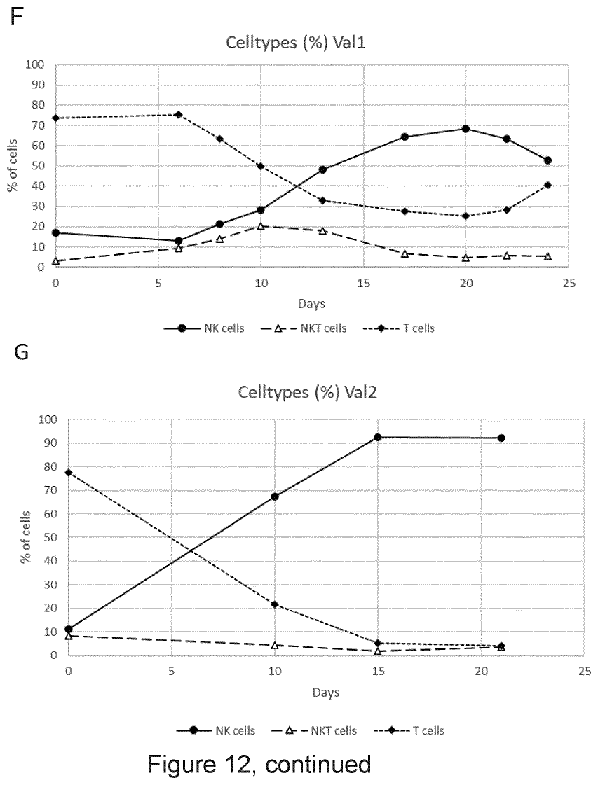

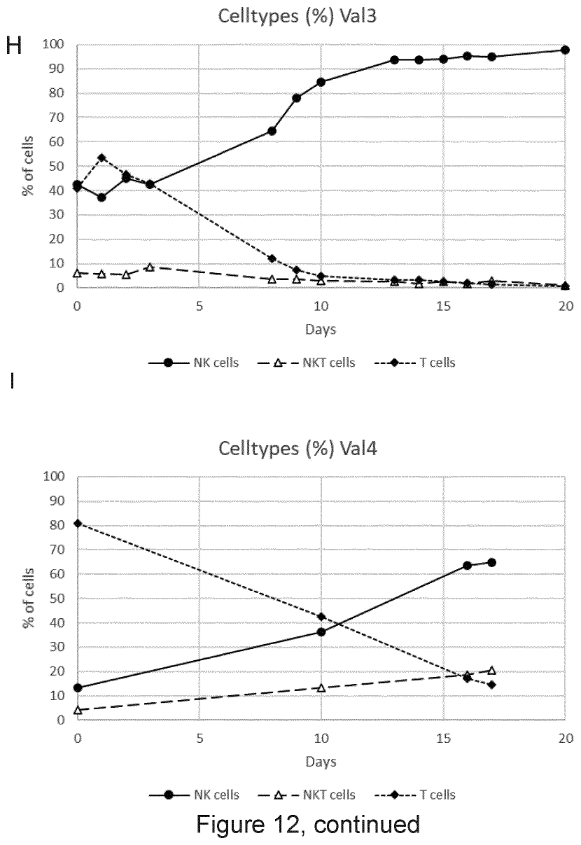

FIG. 12. Bioreactor expansion cultures for validation with cells from three MM patients (Val1-3) and one healthy donor (Val4). The starting concentrations were 0.25×10⁶ cells/mL or lower (see Table 3) and the perfusion was ‘high’ according to Table 1. Fold expansion of total cells (A), glucose and lactate levels (mM) in relation to cell concentration (10⁶ cells/mL) (B, C, D, E), and percentages of NK cells, T cells, and NKT cells (F, G, H, I). NK cells are defined as CD3 CD56⁺, T cells as CD3⁺CD56⁻ and NKT as CD3⁺CD56⁺.

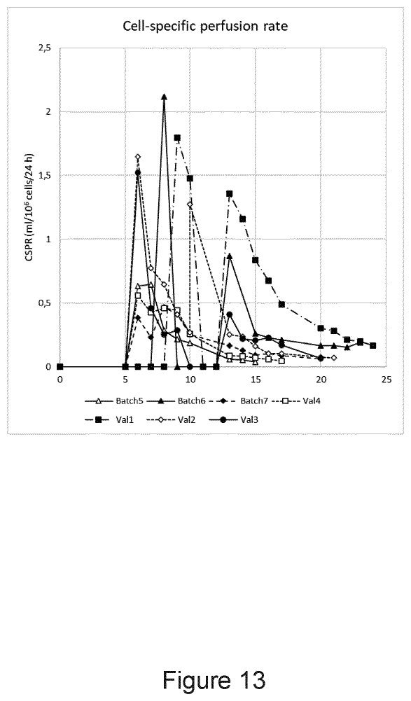

FIG. 13. CSPR in bioreactor expansions with starting concentrations of 0.25×10⁶ cells/mL, calculated as ml fresh media added per million cells per 24 h.

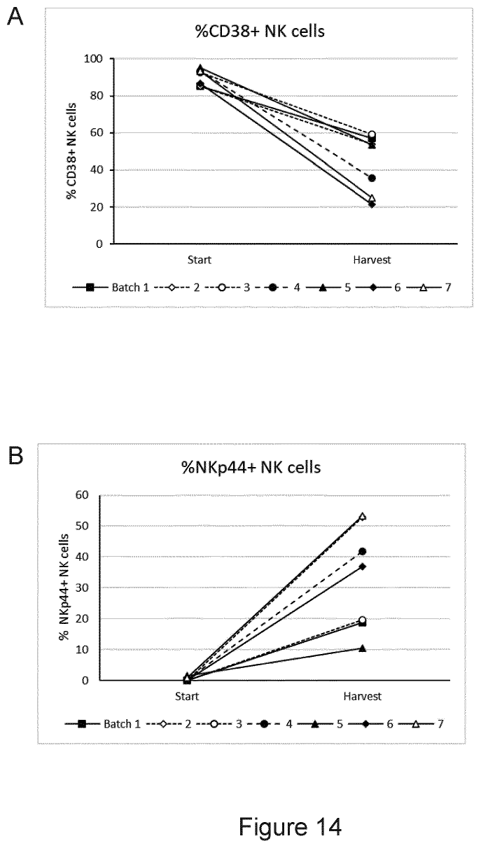

FIG. 14. Expression of CD38 (A) and NKp44 (B) on NK cells before and after expansion in bioreactor cultures with starting concentrations of 0.25×10⁶ cells/mL.

### EXAMPLE 1

### Expansions in Flask Cultures

Natural killer (NK) cell-based adoptive immunotherapy is a promising approach for the treatment of cancer. Ex vivo expansion and activation of NK cells under good manufacturing practice (GMP) conditions are crucial for facilitating large clinical trials. The goal of this study was to optimize a large-scale, feeder-free, closed system for efficient NK cell expansion using lower starting concentrations of PBMCs.

The advantages of a lower starting concentration would be that blood sampling volume could be reduced from fragile patients and avoid previous problems of obtaining high enough cell numbers from the blood samples.

Potential problems could be that reduced cell-cell contacts could affect cell proliferation and activation and that the induction of the expansion cultures relies on activating T cells in order to produce cytokines, mainly IL-2. However, it is unpredictable if the induction will be as effective at lower cell densities in the culture.

### Results—Expansion in Flasks

PBMCs from three healthy donors (HD1, HD2 and HD3), and two multiple myeloma patients (MM) (MM01, MM02) were used to start small-scale cultures at different concentrations (FIG. 1).

For all three healthy donors, the total cell expansion increased with reduced starting concentrations (FIG. 1A, C, E). The expansions of myeloma patient cells cannot be compared directly because the starting concentrations tested were even lower than the ones tested for healthy donors. Notably, all patient-derived cultures expanded well, even the one started with a concentration of 0.15×10⁶ cells/ml (FIG. 1G, I).

Surprisingly, the composition of the product at harvest day differed in relation to the starting concentration in the cultures from healthy donors (FIG. 1B, D, F). For two donors (HD1 and 2), the product contained a higher percentage of NK cells the lower the starting concentration was. There was only a very small difference in product composition for donor HD3. In the expansions of cells from myeloma patient MM01, the highest percentage of NK cells was found in the product started with the lowest concentration (0.15×10⁶ cells/ml) (FIG. 1H), but in the expansions of cells from patient MM02, the product contained smaller fractions of NK cells the lower the starting concentration was (FIG. 1J).

Without being bound by theory, the inventors hypothesise that the reduced NK cell growth at higher cell density could be because there is an increased competition for nutrients or accumulation of inhibitory by-products. To evaluate the turnover of nutrients, glucose and lactate levels were measured for one healthy donor (HD3; FIG. 2A).

High lactate levels were an indication of reduced sub-sequent cell growth (compare lactate day 6 to growth rate day 8) (FIG. 2B).

Surprisingly, CD38 expression was reduced to a lower level when cultures were started at lower seeding densities (FIG. 3).

### Results—Expansions in Bioreactor

The hypothesis that an increased medium exchange could favour the growth of NK cells resulting in higher NK cell percentages in the product at harvest was also supported by observations of our bioreactor expansion cultures, especially when comparing them to the small-scale expansions run in parallel.

Expansions 1 and 2 were a comparison between fresh (1) and frozen (2) starting material from the same donor. Total cell expansion was very similar between the two runs (FIG. 4A). However, the percentage of NK was higher in the expansion from the frozen starting material resulting in a better NK cell yield (FIG. 4B). But neither of the two had an NK cell expansion as the accompanying small-scale culture.

Expansion culture 3 with fresh staring material showed an even higher difference between the bioreactor and the parallel small-scale culture. The small-scale culture contained over 80% NK cells at harvest (FIG. 5B) while the bioreactor culture contained only 19% (FIG. 5C).

The NK cell expansion in the bioreactor slowed down around day 9. The lactate concentration in the bioreactor culture reached levels of over 30 mM by day 7 (FIG. 5D, E). The accumulating lactate and the lower glucose levels indicate that the cells in the bioreactor had a much lower exchange in their culture medium than the cells growing in the flask. Therefore, we considered the cell-specific prefusion rate (CSPR), which is calculated as the volume of fresh medium added per million cells in culture. The difference in the CSPR could be an explanation for the better growth of the cultures in flasks (FIG. 6).

In order to improve the cell growth in expansion culture 4, the perfusion rate was increased (“medium” in Table 1). The total cell expansion was similar to the expansion in the parallel small-scale culture (FIG. 7A). However, the percentage of NK cells was only 25% in the bioreactor culture compared to 57% in the small-scale culture (FIG. 7B, C). The CSPR in the bioreactor was still much lower than in the flask cultures, even though both cultures started with similar rates at day 5 (FIG. 8).

In expansion 5, the perfusion rate was increased further (“high” in Table 1) and the starting cell concentration was reduced to 0.25×10⁶/ml. The total cell expansion was similar between bioreactor and small-scale culture (FIG. 9A). However, importantly the percentage of NK cells at harvest was higher in the bioreactor (FIG. 9B).

Expansions 6 and 7 were performed with the same parameters as expansion 5. In both expansions, the total cell expansion in the bioreactor was lower than in the small-scale cultures (FIG. 10A, B). However, importantly, the percentage of NK cells at harvest day was much higher in the bioreactor (FIG. 10C, F).

The total cell expansion in the three bioreactor cultures with the low starting concentration of 0.25×10⁶ cells/ml and high perfusion rate (expansions 5, 6, and 7) were variable (FIG. 11A). However, importantly the percentage of NK cells in the products of these expansions was higher (FIG. 11B).

Finally, we validated the process with low starting concentration and high perfusion in three expansion cultures with starting material from multiple myeloma (MM) patients (“Val1”, “Val2” and “Val3”) and one with starting material from a healthy donor (“Val4”) (FIG. 12). As in the previous cultures, the expansion of total cells was variable (FIG. 12A). During the third expansion (val3) a technical error led to an interrupted perfusion after day 10 that was fixed on day 13. This disturbed the growth of the cells, but the expansion culture recovered ending with a 90-fold total cell expansion. Nevertheless, all three MM cultures as well as the healthy donor culture had a high percentage of NK cells at harvest (53%, 93%, 98%, and 65%, respectively; FIG. 12F, G, H, I).

In summary, the six bioreactor expansion cultures with a starting concentration of 0.25×10⁶ cells/mL and a high perfusion rate showed total cell expansions between 26- and 211-fold, with a median of 112.4-fold (Table 2, FIG. 11A, and FIG. 12). Importantly, the percentage of NK cells at harvest was between 31 and 98% with a median of 64.7% (Table 2, FIG. 9B, 10C, F, and FIG. 12F, G, H, I).

### DISCUSSION

Despite high inter-donor variability, expansion cultures with a lower starting concentration of PBMCs are feasible with healthy donor cells, as well as MM patient cells. The total cell expansion of the six experimental bioreactor cultures was lower compared to the previous process, however the median percentage of NK cells was higher. This suggests that NK cell growth is favoured by a higher CSPR.

### Materials and Methods

### Small Scale Cultures

Blood cells from three healthy donors (HD1-HD3), and two MM patients (MM01, MM02) were used in NK cell cultures. PBMCs were purified by ficoll gradient separation of “buffy coats” according to manufacturer's protocol. Specifically, anticoagulant-treated blood is carefully layered on a viscous density gradient media according to manufacturer's instructions (Ficoll-Paque (GE healthcare). Specifically, the lymphocytes are then separated from erythrocytes by density-based gradient centrifugation at 800×g, minimum acceleration without break. Cell layer extracted by pipette and washed 3 times in PBS+0.5% HSA at 300×g. The bottom layer contains sedimented, aggregated erythrocytes, the next layer mostly contains granulocytes, the top layer contains plasma and the intermediate layer, and the interface between the plasma and the Ficoll-Paque layer holds PBMCs mostly made up of lymphocytes. The isolated PBMCs are washed several times with phosphate-buffered saline (PBS) containing 0.5% human serum albumin (HSA) and counted.

Separated PBMCs were used as starting material for NK-cell cultures, either directly or after a freeze-thawing cycle, and this is defined as day 0 of culture.

### Expansion in Cell Culture Flasks

Cells were counted and seeded at different concentrations in a final volume of 2 ml/well (6-well plates, Sarstedt), or 8 ml (T-25 bottles Thermo Scientific) of media (SCGM—(Cellgenix)) with 5% human serum (Access Biologicals) and 500 U/ml IL-2 (Novartis); bioreactor cultures also supplemented with 0.1% Pluronic (Gibco/Thermo Fischer Scientific)

Cells were induced with 10 ng/ml anti-CD3 (Miltenyi Biotech) (freeze-thawed samples were induced after overnight recovery, i.e. on day 1 of culture), and kept in culture the following five days. Fresh IL-2 (500 U/ml) was added Monday-Friday, and all cultures were induced on a Wednesday. After the initial 5-day incubation, cells were cultured for a total of 20-22 days and cells were scraped off the bottom and suspended in the culture media to obtain a homogenous mix every two to three days to allow for counting. Culture volume was measured with the pipette, and a small fraction was taken for cell counting with trypan blue. During medium replenishment, the cell concentration was adjusted to 0.5-1×10⁶ cells/mL in each culture. Importantly, all parallel cultures were split to the same concentration, it was only the concentration at culture start that differed. Total cell number was calculated from cell count and measured volume, and cell expansion was calculated as the fold increase of total cell numbers.

Cells were also taken from the culture for FACS analysis of cell types or NK cell activity by degranulation assay, and for glucose and lactate measurements.

### Expansion in Bioreactor

Cells for bioreactor cultures were prepared as above, with the supplement of 0.1% Pluronic F68.

Cultures were started in a volume of 500 ml in a 2-litre bioreactor bag (Cellbag, 2 L Perf, pH, DO (GE healthcare) on a Xuri W25 bioreactor (Cytiva). (Settings: 37° C., rocking at 6 rpm, 6 degrees angle, 5% CO₂ mixed with compressed air, flow rate (flow of compressed air/5% CO₂) 0.1 l/min).

Initial bioreactor cultures in Xuri W25 were started at 0.5×10⁶ cells/ml and followed the feeding regime corresponding to “low” perfusion in Table 1. A small-scale culture in T25 tissue culture flask was grown in parallel as described above. For cultures from fresh PBMCs 10 ng/ml anti-CD3 was directly added to the cell suspension, for freeze-thawed material anti-CD3 was added the following day. Anti-CD3 was prepared with 500 U/ml IL2 in a syringe, and injected into the bioreactor bag through the sample port.

After the five-day induction with anti-CD3, cells were counted daily on weekdays. The bioreactor bag was shaken to dissolve cell aggregates. A sample was withdrawn from the sample port using a 3 ml or 5 ml syringe attached through the luer connection. A fraction was used for cell count by trypan blue or nucleocounter NC-200 (ChemoMetec) with Via-1 cassettes (ChemoMetec). One sample was used for glucose and lactate measurements and remaining cells were frozen for sub-sequent FACS analysis. Cells were diluted daily, based on cell count to keep a constant concentration until the maximal culture volume of 1 litre for 2-litre bioreactor bag was obtained. Different perfusion steps were started when pre-set criteria in terms of cell count and lactate levels were met (Table 1).

TABLE 1
Criteria for four perfusion steps in the three different strategies
used. For the “high” perfusion strategy there was also an
upper limit of 25 mM lactate in cultures prompting increased perfusion.

| Perfusion step | low | | medium | | high | |
|---|---|---|---|---|---|---|
| Target | 0.5M | cells/ml | 0.5M | cells/ml | 0.5M | cells/ml |
| concentration | | | | | | |
| during initial | | | | | | |
| volume increase | | | | | | |
| 1 - 300 ml/day | >3M | cells/ml | >1M | cells/ml | — | |
| 2 - 500 ml/day | >7M | cells/ml | >3M | cells/ml | >1M | cells/ml |
| 3 - 750 ml/day | >15M | cells/ml | >9M | cells/ml | >3M | cells/ml |
| 4 - 1000 ml/day | >25M | cells/ml | >20M | cells/ml | >9M | cells/ml |

TABLE 3
Results for the “high” perfusion strategy.

| | Start of manufacturing | | | Culture | | | | | |
|---|---|---|---|---|---|---|---|---|---|
| | Total | | | | Total | | At harvest | | |
| | cell | Total cell | | | cell | | Fold | | Fold |
| Expansion | number | concentration | % NK | | number | | expansion | % NK | expansion |
| culture | (10⁶) | (10⁶/ml) | cells | Perfusion | (10⁶) | Day | (total) | cells | NK cells |
| 5 | 125 | 0.25 | 12.4% | High | 26 400 | 15 | 211.2 | 30.8% | 524.6 |
| 6 | 125 | 0.25 | 6.4% | High | 3 900 | 23 | 31.2 | 69.0% | 334.8 |
| 7 | 125 | 0.25 | 18.2% | High | 15680 | 20 | 125.4 | 44.5% | 306.7 |
| Val1 | 115 | 0.23 | 16.8% | High | 3021 | 24 | 26.3 | 52.7% | 82.4 |
| Val2 | 125 | 0.25 | 11.2% | High | 14050 | 21 | 112.4 | 92.2% | 952.2 |
| Val3 | 125 | 0.25 | 42.5% | High | 11150 | 20 | 89.2 | 97.9% | 205.5 |
| Val4 | 121 | 0.242 | 13.2% | High | 21700 | 17 | 179.6 | 64.7% | 880.5 |

### FACS Analysis

The cell phenotype and percentage of subpopulations was analysed by flow cytometry. All antibody staining for flow cytometry were done according to the following protocol cells were washed in PBS, stained with LIVE/DEAD fixable Aqua dead cell stain (Invitrogen), and washed again with PBS, resuspended in reagent diluent concentrate 2 (R & D systems) before incubation with antibodies for 30 min 4° C. aCD45-PerCP (clone 2D1), aCD3-PE (SK7), aCD56-APC (NCAM16.2) and aCD38-PE-Cy7 (HIT2) CD14-V500, CD19-V500, NKp44-BB515, NKp30-BV421, CD16-APC-H7 purchased from BD Biosciences, San Jose, CA, USA). Then washed in PBS and fixated in CellFix (BD). The samples were run on a FACS Verse (BD Biosciences, San Jose, CA, USA) and analysed by FlowJo according to Manufacturer's instructions.

### Glucose/Lactate Measurements

Glucose/lactate measurements were carried out on a Biosen instrument (EKF Diagnostic), according to manufacturer's instructions.

### Methods for Expanding Natural Killer Cells (NK Cells)

The present invention relates to methods for expanding and activating a population of natural killer (NK) cells, compositions obtained from those methods, and uses thereof.

Natural killer (NK) cells are increasingly implicated in the therapeutic benefit of established cancer therapies. They represent a promising therapeutic option for patients with various types of malignant disease (Passweg et al., 2006; Rubnitz et al., 2010). One of the most experimented approaches has been adoptive transfer of autologous or allogeneic cytotoxic effectors with tumor cell killing potential to trigger a graft-vs-tumour (GvT) effect. Among the various effector populations that have a potential anti-tumor effect, NK and NK-like T cells stand out with their high cytotoxic capacity (Sutlu T and Alici E., 2009).

Despite notable progress and development of different strategies to optimize the therapeutic value of NK cells, NK cell-based immunotherapy in general had to deal with several challenges, which have limited its efficacy.

NK cells are normally present only in low numbers in peripheral blood mononuclear cells (PBMCs). An option to increase the number and function of donor-derived NK cells is to expand and activate the cells ex vivo before transfer to the patient. Therefore, NK cell expansion protocols are required that not only efficiently induce NK cell proliferation and activate NK cell function but also fulfil regulatory requirements for safety. Furthermore, compounds used during NK cell expansion must not be harmful to the patient. Different protocols have recently been established that claim to meet these requirements and allow the production of NK cells of clinical grade quality. However, the next challenge is the transfer of these protocols to clinical scale in a manageable, Good Manufacturing Practice-compliant (cGMP) way.

Methods have been developed involving GMP-compliant components that allow expansion of polyclonal NK cells in cell culture flasks using PBMCs from healthy donors (Carlens S. et al., 2001 and U.S. Ser. No. 10/242,788), as well as patients with B-cell chronic lymphocytic leukaemia (Guven H. et al, 2003), and multiple myeloma (MM) (Alici E. et al., 2008). These cells have been shown to exert specific cytotoxic activity against fresh human tumour cells in vitro and in experimental models of human tumours (Guimaraes F. et al., 2006) which opens up the possibility to be evaluated in clinical settings. However, the conventional flask-based culture is labour-intensive and cumbersome, thus limiting the cell number that can be handled practically. Previously disclosed protocols (e.g. Miller J S. et al, 1994; Pierson B A. et al, 1996; Luhrn J. et al, 2002; HG Klingemann and J Martinson Cytotherapy. 2004; 6 (1): 15-22) directed to effector cell preparation also include steps such as NK precursor or CD56 separation prior to culture and the use of feeder cells or cGMP-incompatible components. These disadvantages render previous protocols suboptimal and unfeasible to support large clinical studies.

A multitude of necessary hands-on steps complicate the routine use of these scaled-up manual approaches as a standard therapy. For example, expansion of NK cells in cell culture flasks has the inherent risk of exposure to external agents and contamination. Although this risk is minimized in GMP laboratory environments, the use of closed automated systems is preferred as long as it supplies sufficient amounts of cells. In contrast, partial automation of cell cultivation by use of a bioreactor has shown improved NK cell production in large scale, but because such systems require a high cell number to initiate the culture, a manual pre-cultivation step is often necessary until enough cells are generated to start the automated process. Alternatively, large volumes such as apheresis products or whole-unit peripheral blood are needed (Sutlu et al., Cytotherapy, 2010; 12:1044-1055). For example, in relation to the expansion of T cells, a 2 L perfusion Xuri Cellbag bioreactor culture requires at least 3-5×10⁷ mononuclear cells.

However, since NK cells in humans can be found circulating in the peripheral blood at low levels (representing 2-18% of lymphocytes), obtaining large numbers of NK cells to use as a starting material from fragile patients, such as cancer patients, is problematic.

Against this background the inventors have devised a simplified and semi-automated GMP-compatible method for the large-scale expansion and activation of NK cells. In the method presented here, a population of cells (including NK cells) with a concentration of less than 0.5×10⁶ cells/ml of culture media, was sufficient to initiate the current process, and so a manual pre-cultivation step is unnecessary. This is beneficial as it could reduce the blood sampling volume from fragile patients. It is also surprising that NK cell expansion could be achieved from these low starting concentrations as reduced cell-cell contacts are known to affect cell proliferation and activation.

The present method resulted in a highly pure population of NK cells in less than 25 days. The method does not require feeder cells which is advantageous since the presence of residual feeder cells in the final product is of major concern for clinical applications. Surprisingly, CD38 expression in NK cells was also reduced to a lower level in the expanded population relative to the initial population, which may be advantageous in the treatment of cancer such as multiple myeloma (MM).

On average, the method can generate 13.7×10⁹ total cells, comprising 8-9×10⁹ NK cells within a closed culture system within approximately 25 days. Approximately 5×10⁶ to 1×10⁸ NK cells/kg are needed to dose a patient suffering from a cancer such as multiple myeloma, and so this would fall within the range of NK cells typically used in NK cell immunotherapy.

In an aspect, the invention provides a method for expanding a population of natural killer (NK) cells, the method comprising:
i. providing an initial population of cells comprising NK cells;
ii. introducing the initial population of cells into a closed culture system at a concentration of less than 0.5×10⁶ cells/ml of cell culture medium; and
iii. expanding the initial population of cells in the closed culture system, under perfusion conditions effective to produce an expanded population of NK cells, preferably wherein the number of NK cells in the expanded population has increased 50-fold relative to the number of NK cells in the initial population.

By “natural killer cells (NK cells)” we include the meaning of large granular lymphocytes (LGL) that differentiate from the common lymphoid progenitor, like B and T lymphocytes. NK cells comprise 5% to 20% of human peripheral blood lymphocytes and are derived from CD34⁺ hematopoietic progenitor cells. NK cells are known to differentiate and mature in the bone marrow where they then enter the circulation. NK cells differ from natural killer T cells (NKTs) phenotypically, by origin and by respective effector functions; often, NKT cell activity promotes NK cell activity by secreting interferon-γ (IFNγ). In contrast to NKT cells, NK cells do not express the T cell marker CD3 but they usually express the surface markers CD56 in humans. Preferably, the NK cells have the phenotype CD3⁻CD56⁺.

By the terms “expanding”, “expansion” or “proliferation” we include cell growth and multiplication of cell numbers. Expansion or proliferation, as used herein, relate to increased numbers of cells, in particular NK cells, occurring during the culturing process as described herein. The term “culturing process” as used herein refers to the culturing and expansion of NK cells, wherein the starting day (starting point) of the culturing process, i.e. when the initial population of cells is seeded into the closed system, is defined as day 0. The culturing process may last as long as desired by the operator and can be performed as long as the cell culture medium has conditions which allow the cells to survive and/or grow and/or proliferate. Alternatively, the culturing process may continue until certain “release parameters” are met. Such parameters are described below in more detail and in Table 2.

It will be appreciated that when the method of the invention comprises the addition of an NK cell activating agent, the method can be considered to be a method of expanding and activating a population of NK cells. NK cells express many receptors that can activate their cytotoxic and secretory functions. Activated NK cells execute effector functions through different mechanisms. NK cells mediate direct cytotoxicity via the exocytosis pathway with release of cytotoxic granules containing granzymes and perforin, resulting in lysis of the target cell. Additionally, NK cells induce apoptosis of target cells by expression of death receptor ligands, such as Fas ligand or tumour necrosis factor-related apoptosis-inducing ligand (TRAIL). NK cells also modulate the immune response by their ability to produce pro-inflammatory cytokines, most notably Interferon-γ (IFN-γ), Tumour necrosis factor (TNF), granulocyte/monocyte colony-stimulating factor (GM-CSF), and/or chemokines (CCL1, CCL2, CCL3, CCL4, CCL5, and/or CXCL8) which facilitate the activation of T cells and other innate immune cells.

In the context of the present invention by the terms “activation” of NK cells and “activating” NK cells we include the meaning of providing NK cells with an activating signal and/or agent so that the NK cells gain cytotoxic activity. Activating signals and/or agents are discussed herein. Assays to determine whether an NK cell is activated are known in the art and are described herein. The natural cytotoxicity receptors (NCR) (NKp30, NKp44 and NKp46) are among the earliest identified NK cell-activating receptors.

By “initial population of cells” we include the starting material for the expansion which must comprise a proportion of NK cells. In an embodiment, the initial population of cells comprises or consists of NK cells. In an embodiment, the initial population of cells also comprises T cells. Due to variability between individual patient samples, the inventors have observed between 5-20% NK cells in the PBMCs used as the exemplified initial population of cells. In an embodiment, the initial population of cells is obtained from a healthy subject. In an alternative embodiment, the initial population of cells is obtained from a subject in need of cell therapy, such as a subject with cancer.

In an embodiment, step (ii) comprises introducing the initial population of cells into a closed culture system at a concentration of less than 0.5×10⁶ cells/ml, such as less 0.4×10⁶ cells/ml, 0.3×10⁶ cells/ml, 0.25×10⁶ cells/ml, 0.2×10⁶ cells/ml, 0.175×10⁶ cells/ml, 0.15×10⁶ cells/ml, 0.125×10⁶ cells/ml, 0.10×10⁶ cells/ml, 0.075×10⁶ cells/ml, or 0.05×10⁶ cells/ml of cell culture medium. In an embodiment, step (ii) comprises introducing the initial population of cells into a closed culture system at a concentration that does not exceed 0.5×10⁶ cells/ml. In an embodiment, step (ii) comprises introducing the initial population of cells into a closed culture system at a concentration that does not exceed 0.4×10⁶ cells/ml. In an embodiment, step (ii) comprises introducing the initial population of cells into a closed culture system at a concentration that does not exceed 0.3×10⁶ cells/ml. In an embodiment, step (ii) comprises introducing the initial population of cells into a closed culture system at a concentration that does not exceed 0.25×10⁶ cells/ml. In an embodiment, step (ii) comprises introducing the initial population of cells directly into a closed culture system at a concentration between 0.1×10⁶ cells/ml to 0.5×10⁶ cells/ml. In an embodiment, an initial population comprising 0.125×10⁶ cells/ml, or less, is introduced directly into the closed culture system.

As shown in the accompanying Examples, the inventors successfully expanded and activated NK cells even when starting from low concentrations of PBMCs, and without the need to grow the cells in flasks first. This was surprising for several reasons. Firstly, reduced cell-cell contacts were expected to negatively affect cell proliferation and activation. Secondly, the exemplified method comprises activating T cells in order to produce cytokines, mainly IL-2, but it was unknown whether the activation would be as effective at lower cell densities.

Accordingly, step (ii) comprises culture initiation and activation and comprises seeding the initial population of cells directly in a closed culture system comprising a suitable cell culture medium for NK cell expansion. In an embodiment, the initial population of cells was introduced into the closed culture system such that the cell concentration was less than 0.5×10⁶ cells/ml.

By “cell culture medium” we include the meaning of liquids providing the chemical conditions which are required for NK cell maintenance. Examples of chemical conditions which may support NK cell expansion are known in the art and include but are not limited to solutions, buffers, serum, serum components, nutrients, vitamins, cytokines, and other growth factors.

Suitable media for NK cells are: X-Vivo™ serum-free media (BioWhittaker, Verviers, Belgium), AIM VR serum-free medium (Thermo Fisher Scientific, Grand Island, NY, USA), CellGro Stem Cell Growth medium (SCGM) (Cell Genix, Freiburg, Germany), or complete Roswell Park Memorial Institute 1640 (BioWhittaker, Verviers, Belgium). Other media include Glycostem Basal Growth Medium (GBGM®) (Clear Cell Technologies, Beernem, Belgium), which is free of animal-derived components. Media suitable for use to cultivate NK cells as known in the art also includes, TexMACS (Miltenyi), BINKIT NK Cell Initial Medium (Cosmo Bio USA), DMEM/F12, NK Cell Culture Medium (Upcyte technologies).

Steps (ii) and (iii) of the methods described herein can be performed in a closed culture system. By “closed culture system” or “closed system” we include the meaning of a culture vessel and accessory components which reduces the risk of cell culture contamination while performing culturing processes such as the introduction of new material and performing cell culturing steps such as proliferation, differentiation, activation, and/or separation of cells. The vessels and components are utilized without breach of the integrity of the system, permit fluid transfer in and/or out while maintaining asepsis, and are connectable to other closed systems without loss of integrity. Such a system allows to operate under GMP or GMP-like conditions (“sterile”) resulting in cell compositions which are clinically applicable. The Xuri™ Cell Expansion System W25 (Cytivia) is used herein as an exemplary closed system.

By “perfusion conditions” we include the meaning of conditions allowing culture media exchange that do not involve manual handling. During a perfusion culture, waste media containing metabolic products like lactate is withdrawn from the culture and fresh complete medium is added. Accordingly, perfusion conditions allow the continuous feeding of the cells with fresh media and removal of spent media while retaining cells in culture. Typically, during perfusion, there are different ways to keep the cells in culture while removing spent media. One way is to keep the cells in the system by using capillary fibres or membranes to which the cells bind. Another method is to utilize a “lily pad” floating filter that retains the cells in the culture vessel while allowing the media to be removed. For example, a porous polyethylene-based perfusion filter that floats on the medium can be used to retain cells in the culture vessel. Another method relies on gravity settling of cell aggregates which allows for removal of spent media without the use of filtration systems. By continuously removing spent media and replacing it with new media, nutrient levels are maintained for optimal growing conditions and cell waste product is removed to avoid toxicity.

Preferably, the perfusion conditions are conditions of continuous perfusion. By “continuous perfusion” we include the meaning of a constant flow of fresh culture medium into the culture vessel (e.g. bag) while there is an equal flow of cell-free culture supernatant out of the culture vessel (e.g. bag) or as stated above “continuous “feed” of fresh medium into the closed system combined with a continuous outflow of cell free harvest”.

The terms “automated method” or “automated process” as used herein refer to any process being automated through the use of devices and/or computers and computer software which otherwise would or could be performed manually by an operator. Methods that have been automated require less human intervention. In some instances the method of the present invention is automated if at least one step of the present method is performed without any human support or intervention. Alternatively, the method of the present invention is automated if all steps of the method as disclosed herein are performed without human support or intervention. Preferentially the automated process is implemented on a closed system such as the Xuri Cell Expansion System W25.

As used herein the term “culturing” includes providing the chemical and physical conditions (e.g., temperature, gas) which are required for NK cell maintenance. Typically, the cells are incubated at between 23 and 39° C., and preferably at 37° C. Preferably the cells are cultured at a temperature of about 36-40° C., at a CO₂ concentration of about 4.7-5.1%. More preferably, at a temperature of 37° C., at a CO₂ concentration of 5.0%. Often, culturing the cells includes providing conditions for expansion (proliferation). Preferably, step (ii) comprises introducing the initial population of cells into a closed culture system at a concentration of less than 0.5×10⁶ cells/ml in a cell culture medium comprising one or more NK cell activating agent. Examples of chemical conditions which may support NK cell expansion and activation are discussed in detail herein and include those known in the art but are not limited to the use of buffers, serum, nutrients, vitamins, antibiotics, cytokines and/or growth factors which are regularly provided in the cell culture medium suited for NK cell expansion. In an embodiment, the cell culture medium comprises at least one cytokine. GMP-grade cytokines (recombinant human IL-2, IL-15, IL-12, IL-18, and IL-21), antibodies (anti-CD3-OKT3), and other ancillary reagents (nicotinamide-NAM) may serve as medium supplements for NK cell expansion. In an embodiment, the cell culture medium comprises human serum. In an embodiment, the cell culture medium comprises 5% human serum.

Illustrative examples of suitable concentrations of each cytokine or total concentration of cytokines include about 25 U/mL, about 50 U/mL, about 75 U/mL, about 100 U/mL, about 125 U/mL, about 150 U/mL, about 175 U/mL, about 200 U/mL, about 250 U/mL, about 300 U/mL, about 350 U/mL, about 400 U/mL, about 450 U/mL, about 500 U/mL, about 550 U/mL, about 600 U/mL, about 650 U/mL, about 700 U/mL, about 750 U/mL, about 800 U/mL, about 950 U/mL, or about 1000 U/mL or any amount therebetween of cytokine. In an embodiment, the suitable concentrations of each cytokine or total concentration of cytokines is about 500 U/mL.

In an embodiment, the culture medium comprises a non-ionic surfactant. In an embodiment, the non-ionic surfactant is poloxamer, such as poloxamer 188. In an embodiment, the non-ionic surfactant is Pluronic®, such as Gibco® Pluronic® F-68.

In an embodiment, the method produces an expanded cell population which comprises at least 10%, 15% or 20% NK cells, such as at least 21%, 22%, 23%, 24%, 25%, 26%, 27%, 28%, 29% or 30% NK cells. In an embodiment, the method produces an expanded cell population which comprises at least 30% NK cells, such as 35%, 40%, 45%, 50%, 55%, 60%, 65%, 70%, 75% or 80% NK cells. The method is preferably performed until at least about 20%, such as about 30, 40, 50, 60, 70, or 80% of the expanded cell population comprises NK cells. In an embodiment, the expansion is performed until at least about 30% of the expanded cell population comprises NK cells. As shown in the accompanying Examples, the percentage of NK cells at harvest was between 31 and 98% with a median of 61%.

In an embodiment, during the method of the invention the number of NK cells expands 50-fold relative to the number of NK cells in the initial population. The fold expansion of NK cells is calculated as the total cell number at harvest multiplied by the NK-cell percentage at harvest divided by the total cell number at the start of manufacturing multiplied by the NK-cell percentage at the start. During the method the number of NK cells can be increased by at least 2, 3, 4, 5, 6, 7, 8, 9, 10, 15, 20, 25, 30, 35, 40, 45, 50, 60, 70, 75, 80, 90, 100, 110, 120, 125, or 130-fold relative to the initial number of NK cells present at the beginning of the expansion. During the method, the number of NK cells can be increased by at least 200, 220, 240, 260, 280, 300, 320, 340, 360, 380, 400, 420, 440, 460, 480, 500, 520, 540, 560, 580, 600, such as 650, 700, 750, 800, 850, 900, 950 or 1000-fold relative to the number of NK cells present at the beginning of the expansion. In an embodiment, the expansion is performed until the number of NK cells has expanded 50-fold relative to the number of NK cells in the initial population.

As shown in the accompanying Examples, the number of NK cells can be increased by at least 200-fold, such as 210, 220, 230, 240, 250, 260, 270, 280, 290, or 300-fold relative to the number of NK cells present in an initial population from a healthy donor, and at least 50-fold, such as 60, 70, or 80-fold in an initial population from a cancer patient.

Preferably, the method is carried out ex vivo. By “ex vivo” we include the meaning of conditions of treating or performing a procedure on a cell(s), tissue and/or organ which has been removed from a subject's body. The cell(s), tissue and/or organ may be returned to the subject's body in a method of surgery or treatment.

Preferably, the method does not comprise the use of accessory cells as feeder cells, for example antigen-presenting “feeder” cells.

In an embodiment, the method is compatible with Good Manufacturing Practice (GMP) (see EudraLex The Rules Governing Medicinal Products in the European Union Volume 4 Good Manufacturing Practice Guidelines, part IV Good Manufacturing Practice specific to Advanced Therapy Medicinal Products).

As used herein, the term “about” or “approximately” refers to an amount, level, value, number, frequency, percentage, dimension, size, quantity, weight, or length that varies by as much as 15%, 10%, 9%, 8%, 7%, 6%, 5%, 4%, 3%, 2%, or 1% from the referenced amount, level, value, number, frequency, percentage, dimension, size, quantity, weight, or length. In particular embodiments, the term “about” or “approximately” when preceding a value, means a value plus or minus 15%, 10%, 5%, or 1% of the range.

### Preferably Step (ii) Comprises:

(a) introducing the initial population of cells into a closed culture system at a concentration of less than 0.5×10⁶ cells/ml of cell culture medium comprising one or more NK cell activating agent; and
(b) culturing the cells at a concentration that does not exceed about 1×10⁶ cells/ml.

In an embodiment, step (ii) (b) comprises culturing the cells from the initial population at a concentration that does not exceed 1.0×10⁶ cells/ml, such as a concentration that does not exceed 0.9×10⁶ cells/ml, 0.8×10⁶ cells/ml, 0.7×10⁶ cells/ml, 0.6×10⁶ cells/ml, such as 0.5×10⁶ cells/ml, 0.4×10⁶ cells/ml, 0.3×10⁶ cells/ml, 0.25×10⁶ cells/ml, 0.2×10⁶ cells/ml, 0.175×10⁶ cells/ml, 0.15×10⁶ cells/ml, or a concentration that does not exceed 0.125×10⁶ cells/ml.

In a preferred embodiment, during step (ii) the culture volume is increased to the desired volume, such as the operating volume of the culture vessel used. This may be the maximal operating volume permitted in a given culture vessel, which can be determined according to the manufacturer's instructions. By “maximum operating volume” we include the meaning of the maximum volume of cell culture that a given culture vessel can maintain viable cells. For example, the Xuri CellBag 50 L has a maximal operating volume of 25 litres (L), the Xuri CellBag 10 L has a maximal operating volume of 5 L, the Xuri CellBag 2 L has a maximal operating volume of 1 L. Accordingly, it is preferred that step (iii) begins once the culture volume is increased to the maximal operating volume permitted in a given culture vessel.

In a preferred embodiment where the culture volume is increased (for example, to the maximal operating volume permitted in a given culture vessel), during the culture volume increase the cell concentration is increased to, or maintained at, a concentration of less than or equal to 0.5×10⁶ cells/ml.

It is preferred that once the culture volume has increased (for example, to the maximal operating volume permitted in a given culture vessel), the cell concentration is also permitted to increase before perfusion commences in step (iii). Preferred cell concentrations at which perfusion may commence include those provided below and may be a cell concentration of about 1×10⁶ cells/ml.

As shown in the exemplary method, the cells were seeded directly into the bioreactor at 0.25×10⁶ cells/ml and the cell concentration was maintained at or below 0.5×10⁶ cells/ml by adding cell culture media until the culture volume reached 1 L. The cell concentration was then allowed to increase to about 1×10⁶ cells/ml. The method then progressed to step (iii). The inventors surprisingly found that this favours NK cell growth because of reduced competition for nutrients and reduced accumulation of inhibitory by-products.

### Preferably Step (ii) Comprises:

(a) introducing the initial population of cells into a closed culture system at a concentration of less than 0.5×10⁶ cells/ml in a cell culture medium comprising one or more NK cell activating agent; and optionally increasing the cell culture volume to the maximum operating volume of the cell culture vessel while increasing or maintaining the cell concentration at a concentration of less than or equal to 0.5×10⁶ cells/ml; and
(b) culturing the cells at a concentration that does not exceed about 1×10⁶ cells/ml.

In an embodiment, step (ii) comprises culturing the initial population of cells for about 4-12 days, such as about, 4, 5, 6, 7, 8, 9, 10, 11, or 12 days.

Preferably, step (ii) comprises culturing the initial population of cells for about 4-10 days, optionally 5 days.

In an embodiment, step (ii) comprises culturing the initial cell population for a time sufficient to activate one or more of the NK cells and/or for a time sufficient for the cell population to reach a concentration of about 1×10⁶ cells/ml culture medium.

By “in a cell culture medium comprising one or more NK cell activating agent” we include culturing the initial population of cells with one or more NK activating agents in an activation reaction mixture in order to generate (one or more) activated NK cells. The terms “activation” and “activated NK cells” are defined herein. Methods for determining whether an NK cell is activated, or is “cytotoxic” are described herein.

By “one or more NK cell activating agent” we include one or more agents (e.g. antibodies or functional fragments thereof) which are capable of activating NK cells. Examples of NK cell activating agents include molecules which target a T-cell stimulatory, or co-stimulatory, molecule. It will be appreciated that the NK cell activating agent may directly or indirectly activate NK cells. By directly activate NK cells we include the meaning that the agent acts on the NK cell directly (e.g. binds to a molecule on the NK cell surface) to activate it, and by indirectly activate NK cell we include the meaning that the agent activates the NK cell by virtue of having an effect on one or more molecules which in turn activate the NK cell directly. For example, an anti-CD3 antibody can induce secretion of cytokines from T cells which help to activate and expand NK cells. Any combination of one or more NK cell activating agents can be used to produce activated NK cells. Other known NK cell activating agents include antibodies against CD335 (NKp46) and CD2. Preferably, the initial population of cells (such as isolated PBMCs) are activated within the closed system. The one or more activating agents can be added to the culture media of the closed culture system without exposing the initial population of cells (e.g. PBMCs) to the environment. A reaction mixture is typically formed within the chamber of the closed system to perform the activating. The reaction mixture can be formed by adding one or more NK cell activating agents to the cell culture medium. Preferably, the one or more activating agents are used in effective amounts such that activated NK cells are produced.

Various antibodies and functional fragments thereof are known in the art to activate or stimulate NK cells. In an embodiment, the NK cell activating agent is one or more of an anti-CD2, anti-CD335 anti-CD3, and/or an anti-CD28 antibody. In illustrative embodiments, anti-CD3 antibody can be added to the media. Preferably, the NK cell activating agent comprises an anti-CD3 antibody or CD3 binding agent at the following concentrations: about 0.5 ng/ml, about 0.75 ng/ml, about 1 ng/ml, about 2.5 ng/ml, about 5 ng/ml, about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, or about 200 ng/mL, or any intermediate concentration. More preferably, the NK cell activating agent comprises an anti-CD3 antibody or CD3 binding agent at 10 ng/mL.

Preferably, the NK cell activating agent is an anti-CD3 antibody.

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab. Anti-CD3 antibodies also include the UHCT1/UCHT1 clone, also known as T3 and CD3s. Other anti-CD3 antibodies include, for example, otelixizumab, teplizumab, and visilizumab. The term “OKT-3” (also referred to herein as “OKT3”) refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/ml, MACS GMP CD3 pure, Miltenyi Biotech, Inc., San Diego, CA, USA) and muromonab or variants, conservative amino acid substitutions, glycoforms, or biosimilars thereof. As shown the accompanying Examples, an anti-CD3 antibody (OKT-3) and IL-2, can be added to the media during step (ii) (a). In an embodiment, the anti-CD3 antibody is washed out (i.e. removed) from the culture medium during perfusion (i.e. step (iii)).

In an embodiment, GMP-grade monoclonal anti-CD3 antibody (OKT-3) was added at a final concentration of 10 ng/mL.

Activating the initial population comprising NK cells seeded in the closed system may be performed for at least at least 1 hour, at least 5 hours, at least 10 hours, at least 20 hours at least 24 hours, at least 2 days, at least 3 days, at least 4 days, at least 5 days, at least 6 days or any intervening period of time. Preferably, the NK cell activating agent (e.g. an anti-CD3 antibody) is present in the cell culture for 4-6 days, preferably 5 days.

Activation (also termed “induction” herein) may be performed on an initial population of cells comprising freshly isolated cells (e.g. PBMCs) or previously cryopreserved cells. In the event that cryopreserved cells are used, the cells may be thawed and induced after overnight recovery, i.e. on day 1 of culture.

During step (iii) and perfusion, cell number, viability and metabolite levels can be monitored using methods known in art or those described herein. As shown in the accompanying Examples, glucose/lactate measurements were carried out on a Biosen instrument (EKF Diagnostic), according to manufacturer's instructions.

Preferably, the perfusion conditions in step (iii) comprise a perfusion rate effective to maintain lactate levels at 35 mM or less, such as 30 mM or less.

In an embodiment, the perfusion conditions in step (iii) comprise a perfusion rate effective to maintain lactate levels at 30 mM or less, such as at 29 mM, 29 nM, 28 mM, 27 mM, 26 mM, or 25 mM or less, such as at 20 mM, 18 mM, 15 mM, 12 mM, or at 10 mM or less.

By “perfusion rate” we include the meaning of the rate at which culture medium is delivered and removed from the closed culture system. Perfusion rate is expressed as mL medium/24 h. For example, a perfusion rate of 500 ml/24 h equates to the removal and replacement of 500 ml of culture medium in a 24 h period. It will be appreciated that this can be carried out using a non-static perfusion bioreactor system, such as Xuri W25, Xuri W5 (formerly WAVE), Biostat RM, Allegro XRS, ThermoFisher High Performer Rocker.

Preferably, step (iii) is performed until the total number of cells has expanded at least 10-fold relative to the total number of cells in the initial population.

In an embodiment, the expansion is performed until the total number of cells has expanded at least about 10-fold, such as about 20, 30, 40, 50, 60, 70, 80, 90, 100, 110, 120, 130, 140, 150, 160, 170, 180, 190, 200, 210, 220, 230, 240 or at least about 250-fold relative to the total number of cells in the initial population. In an embodiment, the expansion is performed until the total number of cells has expanded at least about 20-fold relative to the total number of cells in the initial population. In an embodiment, the expansion is performed until the total number of cells has expanded at least about 30-fold relative to the total number of cells in the initial population. The total fold expansion of cells is calculated as the total cell number at harvest divided by the total cell number at the start of manufacturing.

As shown in the accompanying Examples, the bioreactor expansion cultures with a starting concentration of 0.25×10⁶ cells/mL and a “high” perfusion rate (Table 1) showed total cell expansions between 26- and 211-fold, with a median of 112-fold.

Preferably, the initial population of cells is selected from the group comprising: a population of peripheral blood mononuclear cells (PBMCs), a population of cells derived from cord blood, a population of cells derived from a cell line, a population of cells derived from primary cells, and a population of cells derived from stem cells. Preferably, the initial population of cells comprises NK cells and T cells.

NK cells can be derived from peripheral blood; umbilical cord blood; stem cells, including induced pluripotent stem cells (IPSCs) and hematopoietic stem cells; and/or NK cell lines (such as NK-92).

NK cells can be obtained from different sources: from the patient (autologous), or from the patient's human leukocyte antigen (HLA)-matched siblings (allogenic), or haploidentical family members or unrelated donors.

The terms “peripheral blood mononuclear cells” or “PBMCs” as used herein refer to any peripheral blood cell having a round nucleus. PBMCs include lymphocytes, such as T cells, B cells, NK cells, and monocytes.

Blood containing PBMCs can be collected or obtained from a subject by any suitable method known in the art. For example, blood can be collected by venipuncture (sometimes referred to as venapuncture, venipuncture or even venu puncture) or any other blood collection method by which a sample of blood and/or PBMCs is collected. PBMCs can be isolated from the peripheral blood of donors via ficoll (GE Healthcare) gradient centrifugation. The volume of blood collected may be between 50 ml and 250 ml, for example, between 75 ml and 125 ml, or between 90 ml and 120 ml, or between 95 and 110 ml. The volume of blood collected can be between 25, 30, 35, 40, 45, 50, 55, 60, 65, 70, 75, 80, 85, 90, 95, 100, 110, 120, 130, 140, 150, 175, 200, 225, 250, 275, 300, 350, 400, 450, 500 ml. Alternatively, volume of blood collected can be between 500, 600, 700, 800, or 900 ml or 1 L.

In the methods disclosed herein, large numbers of NK cells can be generated in a short amount of time, such as 25 days, from a small volume of blood.

In some embodiments, PBMCs can be obtained by apheresis as discussed below.

NK cells may be isolated away from other components of a blood sample in an enrichment step. Such enrichment can be performed, for example, by forming a buffy coat from a blood sample using known methods. The PBMCs can be enriched by collecting the buffy coat and further enriching PBMCs from other blood components using known methods. Enrichment of PBMCs from other blood components and blood cells can be performed, for example, using apheresis, and/or density gradient centrifugation using Lymphoprep™ (STEMCELL Technologies Inc). Neutrophils may be removed before NK cells are processed. An automated apheresis separator may be used which takes blood from the subject, passes the blood through an apparatus that sorts out a particular cell type (such as, for example, PBMCs), and returns the remainder back into the subject. In some embodiments, monocytes and/or macrophages can be removed from the PBMCs using methods known in the art. For example, monocytes and/or macrophages can be removed using magnetic bead activated cell sorting or by allowing the PBMCs to grow on tissue-culture treated surfaces such that the monocytes and/or macrophages adhere, and then transferring the supernatant to a new container.

In embodiment, PBMCs are used as the initial population of cells (i.e. starting material) for NK cell cultures, either directly or after a freeze-thawing cycle.

NK cells can also be generated and/or derived from multipotent progenitor cells and pluripotent stem cells (PSCs). Highly pure, functional NK cells have been generated from CD34⁺ hematopoietic stem cells isolated from umbilical cord blood (UCB). As explained above, the initial population of cells must also comprise T cells.

NK cells can also be generated and/or derived via induced PSCs (IPSCs), as is known to those skilled in the art.

In an embodiment, the initial population of cells is cryopreserved prior to step (ii). Preferably, the cryopreserved initial population of cells is thawed, prior to step (ii).

Methods to thaw cryopreserved NK cells are known in the art. For example, cryopreserved cells can be quickly thawed at 37° C. in a water bath, bead bath, or a commercial controlled thaw rate device and transferred to a container with pre-warmed media. The cells can be washed with media to remove the cryopreservation solution. The cells can be washed with phosphate buffered saline (PBS), optionally supplemented with human serum albumin (HSA), to remove the cryopreservation solution. The cells can be allowed to recover for one or more days. The cells can be used immediately after thawing.

In an embodiment, steps (ii) and (iii) comprise culturing the cell population in the presence of one or more growth factors such as IL-2 and/or IL-15. Preferably, steps (ii) and (iii) comprise culturing the cell population in the presence of Interleukin 2 (IL-2).

Preferably, steps (ii) and (iii) comprise the addition of Il-2. Type I IFN, IL-12, IL-18 and IL-15 are activators of NK cell effector function. It will also be appreciated that IL-2 promotes NK cell proliferation, cytotoxicity and, to some extent, cytokine secretion. The term “IL-2” (also referred to herein as “IL2”) refers to the T cell growth factor known as interleukin-2, and includes all forms of IL-2 including human and mammalian forms, conservative amino acid substitutions, glycoforms, biosimilars, and variants thereof e.g. IL-2 superkine. For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, NH, USA (CELLGRO GMP) or ProSpec-Tany TechnoGene Ltd., East Brunswick, NJ, USA (Cat. No. CYT-209-b) and other commercial equivalents from other suppliers.

The term “IL-15” as used herein refers to cytokine Interleukin-15 and includes all forms of IL-15 including human and mammalian forms, conservative amino acid substitutions, glycoforms, biosimilars, and variants thereof, e.g. IL-15Rα sushi. Preferably, the cytokines are exogenous. Recombinant human IL-15 is commercially available from multiple suppliers, including ProSpec-Tany TechnoGene Ltd., East Brunswick, NJ, USA (Cat. No. CYT-230-b) and ThermoFisher Scientific, Inc., Waltham, MA, USA (human IL-15 recombinant protein, Cat. No. 34-8159-82). In some embodiments, the concentration of IL-15 in the media can be between 10, 15, 20, 25, 30, 35, 40, 45, 50, 60, 70, 80, 90, 100, 125, 150, 200, 250, or 300, 400, 500, 600, 700, 800, 900, or 1,000 U/ml.

In some embodiments, the concentration of IL-2 in the media can be between 10, 15, 20, 25, 30, 35, 40, 45, 50, 60, 70, 80, 90, 100, 125, 150, 200, 250, or 300, 400, 500, 600, 700, 800, 900, or 1,000 U/ml. Preferably, IL-2 is present at 500 U/mL. Preferably, IL-2 is added throughout steps (ii) and (iii). The media can be supplemented with IL-2 multiple times during expansion such as, every 12, 24, 36, or 48 hours. Preferably, IL-2 is continuously added to the closed system.

Preferably, the culture medium in step (ii) and (iii) comprises IL-2 and human serum. More preferably, throughout the culture, the cell culture medium is supplemented with 500 IU/mL IL-2 and 5% human serum. The medium is also supplemented with an agent capable of preventing foam formation, such as a non-ionic surfactant, such as poloxamer, preferably poloxamer 188. In an embodiment, the cell culture medium is Stem Cell Growth medium (Cellgenix) with 5% human serum, 500 U/ml IL-2; and 0.1% pluronic (F-68).

In an embodiment, the perfusion conditions in step (iii) maintain the cell-specific perfusion rate (CSPR) between 0.01-2 mL/10⁶ cells/24 h, such as between 0.02 and 1.5 mL/10⁶ cells/24 h, such as 0.02, 0.05, 0.075, 0.1, 0.5, 0.75, 1.0, 1.25 and 1.5 mL/10⁶ cells/24 h.

By “cell-specific perfusion rate (CSPR)” we include the meaning of the medium exchange rate needed to sustain a given cell population in a perfusion process. Herein, CSPR is expressed as mL culture media added per million cells per 24 hours.

The inventors have surprisingly found that NK cells can be expanded when cell populations having low cell concentrations are perfused. Thus in an embodiment, the perfusion of step (iii) starts when the cell population has a concentration of 1×10⁶ to 2×10⁶ cells/ml of cell culture medium. In an embodiment, the perfusion of step (iii) starts when the cell population has a concentration of less than or equal to 1×10⁶ cells/ml of cell culture medium. Preferably, the perfusion of step (iii) starts when the cell population has a concentration of about 1×10⁶ cells/ml of cell culture medium. It is preferred if the perfusion of step (iii) starts when the cell population has a concentration less than 3×10⁶ cells/ml.

### Preferably the Perfusion Conditions Comprise:

a perfusion rate of about 0.4-0.6× maximal culture vessel operating volume per 24 hours when the cell population has a concentration of between 1×10⁶ cells/ml and 3×10⁶ cells/ml; and/or
a perfusion rate of about 0.6-0.9× maximal culture vessel operating volume per 24 hours when the cell population has a concentration of between 3×10⁶ cells/ml and 9×10⁶ cells/ml; and/or
a perfusion rate of about 0.9-1.2× maximal culture vessel operating volume per 24 hours after the cell population has reached a concentration of at least 9×10⁶ cells/ml.

By “a perfusion rate of about 0.4-0.6× maximal culture vessel operating volume per 24 hours” we include the meaning that in a 24 hour period 0.4-0.6 of the maximal volume of the culture vessel is added to the culture vessel and 0.4-0.6 of the maximal volume of the culture vessel is removed from the culture vessel. By “0.4-0.6× maximal culture vessel operating volume” we include the meaning of a volume of the culture vessel volume that is 40-60% of the maximal operating culture volume in the culture vessel. In other words, if the maximal operating culture vessel volume is 1 L, 0.4-0.6× maximal operating culture vessel volume is 400-600 mL.

The maximal operating culture volume for a given vessel can be determined by the skilled person or will be provided by the manufacturer of the culture vessel. For example, the maximal total volume in a 2 L Xuri Cellbag bioreactor should be 1000 mL, and the maximal total volume in a 10 L Xuri Cellbag bioreactor should be 5000 mL. Preferably, during continuous perfusion, the volume of spent medium removed was equal to the volume of fresh medium added in order to maintain a constant volume. Automated medium exchange and closed-system passaging methods avoid any open steps that could introduce a contaminant, ensuring that the cell product remains sterile.

Examples of perfusion conditions are provided in Table 1. Preferably the perfusion conditions comprise “high” condition as set out in Table 1, which are applicable to a culture vessel with a maximal culture vessel operating volume of 1 L. However, these volumes could be scaled depending on the culture vessel in question.

### The “Low” Perfusion Rate Detailed in Table 1 Comprises:

a perfusion rate of about 0.1-0.4 (preferably about 0.3)× maximal culture vessel operating volume per 24 hours when the cell population has a concentration of between 0.5×10⁶ cells/ml and 3×10⁶ cells/ml; and/or
a perfusion rate of about 0.4-0.6 (preferably about 0.5)× maximal culture vessel operating volume per 24 hours when the cell population has a concentration of between 3×10⁶ cells/ml and 7×10⁶ cells/ml; and/or
a perfusion rate of about 0.6-0.9 (preferably about 0.75)× maximal culture vessel operating volume per 24 hours when the cell population has a concentration of between 7×10⁶ cells/ml and 15×10⁶ cells/ml; and/or
a perfusion rate of about 0.9-1.2 (preferably about 1.0)× maximal culture vessel operating volume per 24 hours after the cell population has reached a concentration of at least 25×10⁶ cells/ml.

### The “Medium” Perfusion Rate Detailed in Table 1 Comprises:

a perfusion rate of about 0.1-0.4 (preferably about 0.3)× maximal culture vessel operating volume per 24 hours when the cell population has a concentration of between 0.5×10⁶ cells/ml and 1×10⁶ cells/ml; and/or
a perfusion rate of about 0.4-0.6 (preferably about 0.5)× maximal culture vessel operating volume per 24 hours when the cell population has a concentration of between 1×10⁶ cells/ml and 3×10⁶ cells/ml; and/or
a perfusion rate of about 0.6-0.9 (preferably about 0.75)× maximal culture vessel operating volume per 24 hours when the cell population has a concentration of between 3×10⁶ cells/ml and 9×10⁶ cells/ml; and/or
a perfusion rate of about 0.9-1.2 (preferably about 1.0)× maximal culture vessel operating volume per 24 hours after the cell population has reached a concentration of at least 20×10⁶ cells/ml.

### For Example, the “High” Perfusion Rate Detailed in Table 1 Comprises:

a perfusion rate of about 0.4-0.6, preferably about 0.5× maximal culture vessel operating volume per 24 hours when the cell population has a concentration of between 1×10⁶ cells/ml and 3×10⁶ cells/ml; and/or
a perfusion rate of about 0.6-0.9, preferably about 0.75× maximal culture vessel operating volume per 24 hours when the cell population has a concentration of between 3×10⁶ cells/ml and 9×10⁶ cells/ml; and/or
a perfusion rate of about 0.9-1.2, preferably about 1.0× maximal culture vessel operating volume per 24 hours after the cell population has reached a concentration of at least 9×10⁶ cells/ml.

In an embodiment in which the closed system is a non-static bioreactor comprising a maximum operating culture volume of 1 L, the “high” perfusion conditions as described in Table 1 comprise:
a perfusion rate of about 400-600 ml (preferably 500 ml) culture medium/24 h when the cell population has a concentration of between 1×10⁶ cells/ml and 3×10⁶ cells/ml; and/or
a perfusion rate of about 600-900 ml (preferably 750 ml) culture medium/24 h when the cell population has a concentration of between 3×10⁶ cells/ml and 9×10⁶ cells/ml; and/or
a perfusion rate of about 900-1200 ml (preferably 1000 ml) culture medium/24 h after the cell population has reached a concentration of at least 9×10⁶ cells/ml.

The skilled person would be able to adjust the conditions above for vessels of different sizes.

In an embodiment in which the closed system is a non-static bioreactor system and a 2 L culture vessel (e.g. 2-litre bioreactor bag) is used with a maximal operating volume of 1 L, the perfusion starts with 500 ml/day when the cells reach a density of 1×10⁶ cells/ml, and after 3×10⁶ cells/ml, to 750 ml/day, and after 9×10⁶ to 1000 ml/day. If the lactate level reaches over 25 mmol/l (i.e. 25 mM), the perfusion rate is increased to the next perfusion rate, as shown in Table 1. In other words, if the lactate level reaches over 25 mmol/l, perfusion is increased.

Preferably, step (iii) comprises expanding the initial cell population for at least 9 days.

The cell expansion step can be performed for a certain number of days. Expansion can 23, 24, or 25 days. In certain embodiments, expansion is performed for between 10 and 24 days, preferably between 12 and 22 days, preferably between 14 and 20 days.

Preferably the method further comprises the step, performed after step (iii), of:
(iv) harvesting the expanded cell population.

By “harvesting the expanded cell population” we include the meaning of terminating the expansion and/or removing the expanded cells from the closed cell system. Harvesting the expanded NK cells can be performed based on an expansion criteria or release criteria. The release criteria for the expanded NK cells herein are shown in the Table 2 below.

TABLE 2
Release criteria

| Parameter | Approval limit |
|---|---|
| Viable cell number | ≥3 × (30 × 10⁶ cells per kg bodyweight) |
| Cell viability | >90% viable cells |
| % NK cells (CD3⁻CD56⁺) | ≥10% of the viable cell population |
| % Activated | >30% CD107a⁺ cells in the CD3⁻CD56⁺ |
| NK cells | population in response to K562 cells |
| Mycoplasma | Not detectable (ND) |
| Sterility | Sterile |
| Endotoxin | ≤0.5 EU/mL |
| Tumour cell contamination | Not detectable (ND) |
| Karyotyping | No clonal aberrations |

The time for expansion can be any suitable time which allows for the production of (i) a sufficient total number of cells in the expanded population; (ii) a population of expanded cells with a favourable proportion of NK cells compared to the initial population (such as at least 10%), or (iii) both (i) and (ii).

Cell harvesting can also be performed a certain number of days after the method started. In some embodiments, harvesting can be performed 7, 8, 9, 10, 11, 12, 13, 14, 15, 16, 17, 18, 19, 20, 21, 22, 23, 24, 25, 26, 27, 28, 29, or 30 days after the method commenced. In some embodiments, harvesting can be performed between 7, 8, 9, 10, 11, 12, 13, or 14 days after the method commenced, or 10, 11, 12, 13, 14, 15, 16, 17, 18, 19, 20, or 21 days after the method commenced, or 15, 16, 17, 18, 19, 20, 21, 22, 23, 24, 25, or 26 days after the method commenced.

In some embodiments, cell harvesting is performed when the NK cells have been expanded at least 2, 3, 4, 5, 6, 7, 8, 9, 10, 11, 12, 13, 14, 15, 16, 17, 18, 19, 20, or 25-fold. For example, cell harvesting may be performed when the NK cells have been expanded at least 5 fold, at least 10 fold, or at least 20 fold. This expansion is typically measured by counting the NK cells, or viable NK cells, harvested after expansion versus the total number of NK cells, or viable NK cells, that were present in the initial cell population (such as PBMCs).

Cell harvesting can also be performed when the total cells reach a specified cell density in the culture media. In some embodiments, harvesting can be performed when the cell density is between 1×10⁵, 2.5×10⁵, 5×10⁵, 1×10⁶, 2.5×10⁶, 5×10⁶, 1×10⁷, 2.5×10⁷, 5×10⁷, 1×10⁸, 2.5×10⁸, 5×10⁸, or 1×10⁹ cells/ml. On average, the method generates 10-14×10⁹ cells (total) and 3-7×10⁹ activated NK cells.

In some embodiments, cell harvesting is performed when the total cells have been expanded at least 2, 3, 4, 5, 6, 7, 8, 9, 10, 11, 12, 13, 14, 15, 16, 17, 18, 19, 20, or 25-fold, relative to the total number of cells in the initial population. For example, cell harvesting may be performed when the total cells have been expanded at least 5 fold, at least 10 fold, or at least 20 fold. Alternatively, cell harvesting is performed when the total cells have expanded at least 20, 30, 40, 50, 60, 70, 80, 90, 100, 110, 120, 130, 140, 150, 160, 170, 180, 190, 200, 210, 220, 230, 240 or at least about 250-fold relative to the total number of cells in the initial population. This expansion is typically measured by counting the total cells, or total viable cells, harvested after expansion versus the total number of cells, or viable cells, that were present in the initial cell population (such as PBMCs).

The harvested (expanded) cells can include different percentages NK cells. The harvested cells can include at least 10%, 15%, 20%, 25%, 30%, 35%, 40%, 45%, 50%, 55%, 60%, 65%, 70%, 75%, 80%, 85%, 90%, 95%, or 100% NK cells. In some embodiments, cell harvesting is performed when the expanded cell population comprises at least 10% NK cells (CD3⁻CD56⁺), such as at least, 15%, 20%, 25%, 30%, 35%, 40%, 45% or at least 50%. Preferably, cell harvesting is performed when the expanded cell population comprises at least 10% NK cells (CD3⁻CD56⁺).

In some embodiments, cell harvesting is performed when the NK cell (CD3⁻CD56⁺) population contains at least 30% activated NK cells (i.e. CD107a⁺ cells in the CD3⁻CD56⁺ population following co-culture with K562 cells), such as at least 35%, 40%, 45%, 50%, 55% or at least 60% activated NK cells in the NK cell population.

As shown in the accompanying Examples, preferably the cells are harvested the earliest at day 15, if the release criteria are met. In some embodiments, at day 10 the NK cells are already activated and show increased degranulation against K562 cells.

In some embodiments, the harvested cells produced by the methods described herein can be cryopreserved at a predetermined dose for use at a later time. Methods and reagents for cryopreserving cells are well-known in the art. Cryopreservation can include one or more washes and/or a step of concentrating the NK cells with a diluent solution, which may be a cryopreservation solution. In some embodiments, the diluent solution can be normal saline, 0.9% saline, PlasmaLyte (Baxter, Lessines, Belgium), 5% dextrose/0.45% NaCl saline solution, human serum albumin (HSA), or a combination thereof.

In some embodiments, human serum albumin (HSA) can be added to the washed and concentrated cells for improved cell viability and cell recovery after thawing. In some embodiments, the washing solution can be PBS and washed and concentrated cells can be supplemented with HSA, for example 1%, 2%, 3%, 4%, 5%, 6%, 7%, 8%, 9%, or 10% HSA. The method can also include a step of forming a cryopreservation mixture, which includes the NK cells in the diluent solution and a suitable cryopreservation solution.

The cryopreservation solution can be any suitable cryopreservation solution including, but not limited to, CryoStor10 (BioLife Solution), mixed with the diluent solution of NK cells at a ratio of 1:1 or 2:1. The cryopreservation solution can include at least 1%, 2%, 3%, 4%, 5%, 6%, 7%, 8%, 9%, or 10% DMSO. HSA can be added to a final concentration in the cryopreservation solution of between 1%, 2%, 3%, 4%, 5% 6%, 7 8%, 9 10%, 11%, 12%, 13%, 14%, or 15% HSA. The method can include a step of freezing the cryopreservation mixture. In one aspect, the cryopreservation mixture is frozen in a controlled rate freezer using a defined freeze cycle. The method can include a step of storing the cryopreservation mixture in vapor phase liquid nitrogen or liquid nitrogen. The cells can be cryopreserved in a cryopreservation solution comprising human plasma with 5% DMSO.

In certain embodiments of the methods disclosed herein, the harvested NK cells can be introduced, reintroduced, infused, or reinfused into a subject in need of NK cell therapy for a therapeutic effect. The number of NK cells to be reintroduced can be a predetermined dose, which can be a therapeutically effective dose as provided below. The predetermined dose can depend on the nature of the disease or condition being treated. The predetermined dose of harvested cells may be based on the mass of a subject, for example, cells per kilogram of the subject (cells/kg). In any of the embodiments disclosed herein, the number of NK cells to be introduced, reintroduced, or infused back into a subject can be between 1×10³, 2.5×10³, 5×10³, 1×10⁴, 2.5×10⁴, 5×10⁴, 1×10⁵, 2.5×10⁵, 5×10⁵, 1×10⁶, 2.5×10⁶, 5×10⁶, or 1×10⁷, 2×10⁷, 3×10⁷, 5×10⁷, or 1×10⁸ cells/kg. In an embodiment, 5×10⁶ to 1×10⁸ cells/kg are given to a subject.

A subject can be, for example, an animal, a mammal, and a human. In some embodiments, the subject can be healthy. In other embodiments, the subject can be suffering from a disease or condition, such as cancer. Suitable subjects include any individual, e.g., a human or non-human animal who has cancer, who has been diagnosed with cancer, who is at risk for developing cancer, who has had cancer and is at risk for recurrence of the cancer, who has been treated with an agent for the cancer and failed to respond to such treatment, or who has been treated with an agent for the cancer but relapsed after initial response to such treatment.

In an embodiment, the closed culture system is a non-static perfusion bioreactor.

By “non-static perfusion bioreactor” we include the meaning of a system in which cells, cell culture medium, chemicals and reagents are aseptically added, removed and/or manipulated without breach of integrity of the system (e.g., by opening the cap of a tube or lifting the lid off a cell culture plate or dish). Single-use or multiple-use bags and/or containers and/or are added onto or into the bioreactor system for example by sterile tube welding at the site of the vessel or bioreactor. An exemplary bioreactor system is the Xuri™ W25 Cell Expansion System (Cytiva). The platform software provides the ability to perform continuous or discontinuous perfusion or medium exchange in a closed system, with datalogging of all aspects of cell culture, including CO₂ levels, temperature, bag weight, pump speeds, and gas exchange. Optical sensors are available for continuous monitoring of dissolved oxygen and pH, with real-time controls and data storage.

In an embodiment, the bioreactor system comprises means to control evaporation and/or condensation; means to control accumulation of waste metabolites; means to control temperature; means to control the gas flow rate; and/or means to control addition of culture medium or other additives.

In an embodiment, the bioreactor system comprises a computer terminal, a bioreactor control unit, a gas outlet line, a sensor cable, a cell expansion system, a rocking platform, a bioreactor, a gas inlet port, a sensor for measuring oxygen uptake, a media waste line, a media feed line, and pump unit, a media feed reservoir, and a media waste reservoir. The pump unit, the cell expansion system and bioreactor control unit may be connected to the computer terminal which controls the rate that the pump unit operates which controls the rate of media exchange in response to oxygen uptake readings.

Preferably, the closed culture system comprises a culture vessel positioned on a platform capable of rocking. In an embodiment, the closed system is a rocking-motion bioreactor system such as Xuri W25, Xuri W5 (formerly WAVE), Biostat RM, Allegro XRS, ThermoFisher High Performer Rocker.

By “culture vessel” we include the meaning of any sterile vessel or chamber suitable for expanding NK cells. In an embodiment, the culture vessel is single use. In an embodiment, the culture vessel comprises a cell retention means, capable of retaining cells whilst allowing culture media exchange via perfusion. Preferably, the culture vessel has low gas permeability. The culture vessel may comprise an air inlet filter; air outlet filter; needleless sampling port; inoculation/harvest lines; optical dissolved oxygen (DO) sensor; optical pH sensor; perfusion filter; screw cap; and/or aseptic connectors.

The size of the culture vessel should match the medium volume for ideal fluid motion, and can be selected by the person skilled in the art. A culture vessel described herein may have a volume of from about 50 ml to about 100 litres, about 100 ml to about 100 litres, about 150 ml to about 100 litres, about 200 ml to about 100 litres, about 250 ml to about 100 litres, about 500 ml to about 100 litres, about 1 litre to about 100 litres, about 1 litre to about 75 litres, about 1 litre to about 50 litres, about 1 litre to about 25 litres, about 1 litre to about 20 litres, about 1 litre to about 15 litres, about 1 litre to about 10 litres, about 1 litre to about 5 litres, about 1 litre to about 2.5 litres, or about 1 litre to about 2 litres.

Preferably, the bioreactor system comprises a flexible cell bag. Preferably, the flexible cell bag is a sterile bag.

The size of the cell bag should match the medium volume for ideal fluid motion, and can be selected by the person skilled in the art. A cell bag described herein may have a volume of from about 50 ml to about 100 litres, about 100 ml to about 100 litres, about 150 ml to about 100 litres, about 200 ml to about 100 litres, about 250 ml to about 100 litres, about 500 ml to about 100 litres, about 1 litre to about 100 litres, about 1 litre to about 75 litres, about 1 litre to about 50 litres, about 1 litre to about 25 litres, about 1 litre to about 20 litres, about 1 litre to about 15 litres, about 1 litre to about 10 litres, about 1 litre to about 5 litres, about 1 litre to about 2.5 litres, or about 1 litre to about 2 litres.

For example, a 1 L cell bag can be used for medium volumes between 150 and 500 mL, while a 2 L cell bag can be used for medium volumes between 400 ml and 1 L. The same parameters can be applied for both the 1 and 2 L cell bags, as the bag size scales appropriately with volume depth. Cell bags are typically available at 1, 2, 10, 20 and 50 L sizes for scalable cell culture. Optionally other Cell bags and/or vessels of larger or smaller volume can be employed for the methods described herein. In an illustrative embodiment, the cell bag is a Xuri™ CellBag.

In an embodiment, the closed system comprises a rocking motion bioreactor system. For a rocking bioreactor system, two parameters are the rock angle (maximal angle as measured from a flat resting position) and the rock speed (in rocks per minute). The speed and angle of the rocking platform are variable in order to balance oxygenation and mixing of the culture media, and shear forces.

Bioreactor systems allow for different rocking rates and a variety of different rocking angles. Exemplary rocking rates include, but are not limited to, 1, 2, 3, 4, 5, 6, 7, 8, 9, 10, 11, 12, 13, 14, 15, 16, 17, 18, 19, or 20 rocks per minute (rpm). In certain embodiments, the rocking platform angle is set at 1.5°, 2°, 2.5°, 3°, 3.5°, 4°, 4.5°, 5°, 5.5°, 6°, 6.5°, 7°, 7.5°, 8°, 8.5°, or 9.0°. The rocking angle and speed needed for a given culture vessel can be determined by using combinations of rock angle and speed, monitoring dissolved oxygen, and observing the cell count and composition of the cell population.

Preferably, the closed culture system is non-static and is maintained at a rocking rate of 4-8 rocks per minute (rpm), preferably 6 rpm. This is preferably during step (iii).

Preferably the closed culture system is maintained at an angle between 4-8 degrees, preferably 6 degrees. This is preferably during step (iii).

Preferably the closed culture system is maintained at a rocking rate of 4-8 rpm and at an angle between 4-8 degrees, preferably 6 degrees. This is preferably during step (iii).

More preferably, during step (iii) the closed culture system is maintained at a rocking rate of 6 rpm and at an angle of 6 degrees.

Preferably, the NK cells in the expanded population have increased cytotoxicity relative to NK cells in the initial population of cells.

In an embodiment, the expanded population of NK cells produced during step (iii) has increased cytotoxicity relative to the initial population of cells comprising NK cells of step (i). The inventors have observed that NK cell activity is higher in the expanded population relative to the initial population. Therefore, the NK cell product produced by the described method is highly cytotoxic.

Preferably, the cytotoxicity of the NK cells is as determined by an in vitro cytotoxicity test. A skilled person can determine the cytotoxicity using available methods. One way of determining if cells exhibit an increased cytotoxicity is to use the in vitro analysis of cell mediated cytotoxicity against K562 cells using the standard 4 hour ⁵¹Cr-release assay, at an effector cell to target cell ratio of 10:1. Alternatively, a degranulation assay can be used. For example, a degranulation assay against K562 cells, followed by measuring the percentage of degranulated cells in each lymphocyte subpopulation. Both of these assays are described in Sutlu et al., Cytotherapy, 2010; 12:1044-1055 and WO 2010/110734 (see “3. Evaluation of cell mediated cytotoxicity”). Other in vitro cytotoxicity tests are known in the art.

As shown in the accompanying Examples, the inventors have observed that the expanded cell populations from initial populations of cells with concentrations of 0.25×10⁶/ml contained 10.5%, 37%, and 53.4%, NKp44-positive NK cells. The fraction of cells that responded with degranulation during incubation with the cell line K562 was expression level of the activation marker generally correlates with functional response, both measures of activation can differ.

In an embodiment, the proportion of NK cells expressing surface CD38 in the expanded population is less than 70%.

In an embodiment, the proportion of NK cells expressing surface CD38 in the expanded population may be less than 70%, such as less than 60%, 50%, 40%, 30%, 20%, or less than 10%.

CD38 is a type II 45 kDa glycoprotein usually present in the cellular surface membrane. CD38 has multifaceted roles since it possesses properties of an activation marker, an adhesion molecule interacting with endothelial CD31 and ecto-enzymatic activity. CD38 is also an intracellular signaling protein. CD38 is an established immunotherapeutic target in multiple myeloma and under investigation as a target antigen in acute myeloid leukaemia (AML). CD38 expression on NK cells and its further induction during certain ex vivo NK cell expansion methods represents a problem to the development of anti-CD38 therapies when combined with NK cell therapy. Some groups have used CRISPR/Cas9 to knockout CD38 in primary NK cells (Kararoudi et al. Blood 2020). It will be appreciated that such approaches will knockout intracellular and extracellular CD38. The inventors have surprisingly found that the fractions of CD38-positive cells before and after Expansions 6, 7 and 9 were 95.2% and 53.6%; 86.8% and 21.6%; and 93.3% and 25%, respectively. The reduction in surface CD38 expression in the expanded NK cells using the claimed methods is advantageous because genetic manipulation, for example by CRISPR/Cas9, is not required and so the cells have no risk of genetic off-target effects and also would not be classified a genetically modified organisms (GMO). In other words, the inventors observed a down regulation of surface CD38 that is independent of genetic engineering. In an embodiment, the expanded cell population comprising NK cells has not undergone genetic engineering.

CD38 expression can be measured by any method known in the art such as FACS and western blot.

Preferably, the proportion of NK cells in the expanded population that express surface CD38 is less than the proportion of cells in the initial population that express surface CD38.

By “the proportion of NK cells in the expanded population that express surface CD38 is less than the proportion of cells in the initial population that express surface CD38” we include the meaning of that the proportion of NK cells expressing surface CD38 in the expanded population is less than the proportion of NK cells expressing surface CD38 in the initial population.

In another aspect, the invention provides a population comprising activated natural killer (NK) cells with the phenotype CD3⁻CD56⁺ obtainable or obtained by a method described herein.

In another aspect, the invention provides a population comprising activated natural killer (NK) cells with the phenotype CD3⁻CD56⁺, wherein less than 70% of the NK cells express surface CD38, optionally which are obtainable or obtained by a method described herein.

In another aspect, the invention provides a pharmaceutical composition comprising a population of activated NK cells as defined herein and a pharmaceutically acceptable, diluent, carrier, or excipient.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyl oleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavouring agents, dyes, fluid and nutrient replenishers, such like materials and combinations thereof, as would be known to one of ordinary skill in the art. The pH and exact concentration of the various components in a pharmaceutical composition are adjusted according to well-known parameters.

Liquid pharmaceutical compositions, whether in solution, suspension, or other similar form, may comprise one or more of the following: DMSO, sterile diluents such as water for injection, saline solution (preferably physiological saline), ringer's solution, isotonic sodium chloride, fixed oils (such as synthetic mono- or diglycerides which may serve as a solvent or suspending medium), polyethylene glycols, glycerol, propylene glycol or other solvents; antibacterial agents such as benzyl alcohol or methyl paraben; antioxidants, such as ascorbic acid or sodium bisulfite; chelating agents, such as ethylenediaminetetraacetic acid; buffers such as acetate, citrate or phosphate; and agents for adjusting tonicity, such as sodium chloride or glucose.

In some embodiments, cells are formulated in a unit dosage injectable form, such as a solution, suspension, or emulsion. Pharmaceutical formulations suitable for injection of cells typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof. The skilled artisan can readily determine the amount of cells and optional additives, vehicles, and/or carrier in compositions to be administered in methods of the invention.

In another aspect, the invention provides a population of activated NK cells, or the pharmaceutical composition as described herein, for use in medicine.

In another aspect, the invention provides a population of NK cells as described herein, or a pharmaceutical composition as described herein, for use in a method of adoptive cell therapy.

In another aspect, the invention provides a method for performing adoptive cell therapy comprising administering a population of NK cells as described herein, or a pharmaceutical composition as described herein to a patient in need thereof.

In another aspect, the invention provides use of a population of NK cells as described herein, or a pharmaceutical composition as described herein, in the manufacture of a medicament for adoptive cell therapy.

By “adoptive cell therapy” we include a type of immunotherapy in which NK are given to a patient to help the body fight diseases, such as cancer. Adoptive cell therapy can also be termed adoptive cell transfer, cellular adoptive immunotherapy, and NK-cell transfer therapy.

The term “autologous” refers to any material derived from the same individual to which it is later to be re-introduced. For example, cell therapy may comprise collection PBMCs from a donor, e.g., a patient, which are then expanded, and then administered back to the same donor, e.g., patient.

The term “allogeneic” refers to any material derived from one individual which is then introduced to another individual of the same species, e.g., allogeneic NK cell transplantation.

In an embodiment, the patient receives a therapeutically effective amount of NK cells. By a “therapeutically effective amount” or “therapeutically effective dosage,” we include an amount of the NK cells that are produced by the present methods and that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of the NK cells to promote disease regression can be evaluated using a variety of methods known to the skilled practitioner, such as in human subjects during clinical trials, in animal model systems predictive of efficacy in humans, or by assaying the activity of the agent in in vitro assays.

Compositions can be administered in dosages and by techniques well known to those skilled in the medical and veterinary arts taking into consideration such factors as the age, sex, weight, and condition of the particular patient, and the formulation that will be administered (e.g., solid vs. liquid).

It is to be appreciated that a single dose may be delivered all at once, fractionally, or continuously over a period of time. The entire dose also may be delivered to a single location or spread fractionally over several locations.

Cells may be administered in an initial dose, and thereafter maintained by further administration. Cells may be administered by one method initially, and thereafter administered by the same method or one or more different methods. The levels can be maintained by the ongoing administration of the cells. Suitable regimens for initial administration and further doses or for sequential administrations may all be the same or may be variable. Appropriate regimens can be ascertained by the skilled artisan, from this disclosure, the documents cited herein, and the knowledge in the art. The dose, frequency, and duration of treatment will depend on many factors, including the nature of the disease, the subject, and other therapies that may be co-administered.

A variety of other therapeutic agents may be used in combination with the compositions described herein.

In another aspect, the invention provides a population of NK cells as described herein, or a pharmaceutical composition as defined herein, for use in the treatment and/or prevention of cancer and/or a viral infection in a patient.

In another aspect, the invention provides use of a population of NK cells as defined herein, or a pharmaceutical composition as defined herein, in the manufacture of medicament for treating and/or preventing cancer and/or a viral infection in a patient.

In another aspect, the invention provides a method of treating and/or preventing cancer and/or a viral infection in a patient comprising administering a population of NK cells as defined herein, or a pharmaceutical composition as defined herein to the patient.

As used herein, “treatment” or “treating” includes any beneficial or desired effect on the symptoms or condition of a disease or pathological state, and may even include a minimal reduction in one or more measurable markers of the disease or condition being treated (e.g., cancer). Treatment may optionally include amelioration or complete alleviation of one or more symptoms of the disease or condition, or delay in the progression of the disease or condition. “treatment” does not necessarily indicate complete eradication or cure of the disease or condition or symptoms associated therewith.

As used herein, “prevent” and similar words such as “prevent”, “preventing”, and the like refer to methods for preventing, inhibiting, or reducing the likelihood of occurrence or recurrence of a disease or condition (e.g., cancer). It also refers to delaying the onset or recurrence of a disease or condition, or delaying the onset or recurrence of symptoms of a disease or condition. As used herein, “prevention” and similar words also include reducing the intensity, impact, symptoms, and/or burden of a disease or condition prior to its onset or recurrence.

A “patient” as used herein includes any mammal who is afflicted with a disease such as cancer (e.g., a lymphoma or a myeloma). The mammal may be any domestic or farm animal. Preferably, the mammal is a rat, mouse, guinea pig, cat, dog, horse, or a primate. Most preferably, the mammal is human. The terms “subject” and “patient” are used interchangeably herein. The term “donor subject” refers to herein a subject whose cells are being obtained for expansion. The donor subject can be a cancer patient that is to be treated with a population of cells generated by the methods described herein (i.e., an autologous donor), or can be an individual who donates a sample that, upon generation of the population of cells generated by the methods described herein, will be used to treat a different individual or cancer patient (i.e., an allogeneic donor). Those subjects who receive the cells that were prepared by the present methods can be referred to as “recipient subject.”

“Administering” refers to the physical introduction of an agent to a subject, using any of the various methods and delivery systems known to those skilled in the art. Exemplary routes of administration for the cell product prepared by the methods disclosed herein include intravenous, intramuscular, subcutaneous, intraperitoneal, spinal, or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Preferably, the cell product is administered via infusion into the antecubital vein of the left or right arm. Administering can also be performed, for example, once, a plurality of times, and/or over one or more extended periods.

A “cancer” refers to a broad group of various diseases characterized by the uncontrolled growth of abnormal cells in the body. Unregulated cell division and growth results in the formation of malignant tumours that invade neighbouring tissues and can also metastasize to distant parts of the body through the lymphatic system or bloodstream. A “cancer” or “cancer tissue” can include a tumour at various stages.

In a preferred embodiment, the cancer is a haematological cancer. By “haematological cancer” we include types of cancer affecting blood, bone marrow and lymph nodes, such as those selected from the group comprising or consisting of: myeloma, lymphoma, leukaemia, and chronic myeloproliferative diseases.

An article of manufacture or a kit comprising expanded NK cells is also provided herein. The article of manufacture or kit can further comprise a package insert comprising instructions for using the immune cells to treat or delay progression of cancer in an individual or to enhance immune function of an individual having cancer. Suitable containers include, for example, bottles, vials, bags, and syringes. The container may be formed from a variety of materials such as glass, plastic (such as polyvinyl chloride or polyolefin), or metal alloy (such as stainless steel or hastelloy). The container may hold the formulation and the label on, or associated with, the container may indicate directions for use. The article of manufacture or kit may further include other materials desirable from a commercial and user standpoint, including other buffers, diluents, filters, needles, syringes, and package inserts with instructions for use. The article of manufacture further may include one or more of another agent (e.g., a chemotherapeutic agent). Suitable containers for the one or more agent include, for example, bottles, vials, bags and syringes.

In another aspect, the invention provides method, a population of NK cells, a population of NK cells for use, use of a population of NK cells, pharmaceutical composition, substantially as described herein with reference to the accompanying claims, drawings, and examples.

The present invention will now be described with reference to the following non-limiting Figures and Examples.

FIG. 1. Total cell expansion at different starting concentrations in three small-scale cultures of healthy donor PBMC (A, C, E) and two of MM patient PBMC (G, I). (B, D, F, H J) Lymphocyte composition in the respective cultures, as % of CD45⁺, CD19⁻ and CD14 populations. NK cells are defined as CD3⁻CD56⁺, T cells as CD3⁺CD56⁻ and NKT as CD3⁺CD56⁺. Values are average and standard deviation of duplicate cultures.

FIG. 2. (A) Glucose and lactate levels (mM) in cultures with different starting concentrations of HD6 and (B) growth rate of the cell culture. The growth rate is calculated as expansion per 24 h.

FIG. 3. CD38 expression on NK cells. (A) NK cell CD38 expression at harvest compared to the expression at culture start for HD6. (B, C) NK cell CD38 expression at different time points during culture of cells from myeloma patient MM01 (B) and MM02 (C)

FIG. 4. Comparison of bioreactor expansions 1 (fresh) and 2 (frozen). The perfusion was ‘low’ according to Table 1. Fold expansion of total cells (A), fold expansion of NK cells (B), and glucose and lactate levels in relation to cell concentration (C, fresh material; D, frozen material).

FIG. 5. Bioreactor expansion 3 in comparison to expansion in culture flasks. The perfusion was ‘low’ according to Table 1. Fold expansion of total cells (A), percentages of NK cells, T cells, and NKT cells in flasks (B) and bioreactor (C), and glucose and lactate levels in relation to cell concentration in the bioreactor (D) and flask culture (E). NK cells are defined as CD3⁻CD56⁺, T cells as CD3⁺CD56⁻ and NKT as CD3⁺CD56⁺.

FIG. 6. Cell Specific Perfusion Rate (CSPR) in expansion 3 calculated as ml fresh media added per million cells per 24 h.

FIG. 7. Bioreactor expansion 4 in comparison to expansion in culture flasks. The perfusion regime was changed to ‘medium’ according to Table 1. Fold expansion of total cells (A), percentages of NK cells, T cells, and NKT cells in flasks (B) and bioreactor (C), and glucose and lactate levels in relation to cell concentration in the bioreactor (D) and the flask culture (E).

FIG. 8. Cell specific perfusion rate for expansion 4 calculated as ml fresh media added per million cells per 24 h.

FIG. 9. Bioreactor expansion 5 with ‘high’ perfusion according to Table 1 in comparison to expansion in culture flasks. The starting concentration was 0.25×10⁶ cells/mL. Fold expansion of total cells (A), percentages of NK cells, T cells, and NKT cells (B), and glucose and lactate levels (mM) in relation to cell concentration (10⁶ cells/mL) in bioreactor (C) and flask culture (D).

FIG. 10. Bioreactor expansions 6 and 7 in comparison to the respective flask cultures. The starting concentration was 0.25×10⁶ cells/mL and perfusion ‘high’ according to Table 1. Fold expansion of total cells in expansion 6 (A) and 7 (B), glucose and lactate levels (mM) in relation to cell concentration (10⁶ cells/mL) (D and E)), and percentages of NK cells, T cells, and NKT cells (C and F).

FIG. 11. Comparison of total cell expansion and percentage of NK cells in all seven bioreactor expansion cultures with healthy donor cells.

FIG. 12. Bioreactor expansion cultures for validation with cells from three MM patients (Val1-3) and one healthy donor (Val4). The starting concentrations were 0.25×10⁶ cells/mL or lower (see Table 3) and the perfusion was ‘high’ according to Table 1. Fold expansion of total cells (A), glucose and lactate levels (mM) in relation to cell concentration (10⁶ cells/mL) (B, C, D, E), and percentages of NK cells, T cells, and NKT cells (F, G, H, I). NK cells are defined as CD3 CD56⁺, T cells as CD3⁺CD56⁻ and NKT as CD3⁺CD56⁺.

FIG. 13. CSPR in bioreactor expansions with starting concentrations of 0.25×10⁶ cells/mL, calculated as ml fresh media added per million cells per 24 h.

FIG. 14. Expression of CD38 (A) and NKp44 (B) on NK cells before and after expansion in bioreactor cultures with starting concentrations of 0.25×10⁶ cells/mL.

### EXAMPLE 1

### Expansions in Flask Cultures

Natural killer (NK) cell-based adoptive immunotherapy is a promising approach for the treatment of cancer. Ex vivo expansion and activation of NK cells under good manufacturing practice (GMP) conditions are crucial for facilitating large clinical trials. The goal of this study was to optimize a large-scale, feeder-free, closed system for efficient NK cell expansion using lower starting concentrations of PBMCs.

The advantages of a lower starting concentration would be that blood sampling volume could be reduced from fragile patients and avoid previous problems of obtaining high enough cell numbers from the blood samples.

Potential problems could be that reduced cell-cell contacts could affect cell proliferation and activation and that the induction of the expansion cultures relies on activating T cells in order to produce cytokines, mainly IL-2. However, it is unpredictable if the induction will be as effective at lower cell densities in the culture.

### Results—Expansion in Flasks

PBMCs from three healthy donors (HD1, HD2 and HD3), and two multiple myeloma patients (MM) (MM01, MM02) were used to start small-scale cultures at different concentrations (FIG. 1).

For all three healthy donors, the total cell expansion increased with reduced starting concentrations (FIG. 1A, C, E). The expansions of myeloma patient cells cannot be compared directly because the starting concentrations tested were even lower than the ones tested for healthy donors. Notably, all patient-derived cultures expanded well, even the one started with a concentration of 0.15×10⁶ cells/ml (FIG. 1G, I).

Surprisingly, the composition of the product at harvest day differed in relation to the starting concentration in the cultures from healthy donors (FIG. 1B, D, F). For two donors (HD1 and 2), the product contained a higher percentage of NK cells the lower the starting concentration was. There was only a very small difference in product composition for donor HD3. In the expansions of cells from myeloma patient MM01, the highest percentage of NK cells was found in the product started with the lowest concentration (0.15×10⁶ cells/ml) (FIG. 1H), but in the expansions of cells from patient MM02, the product contained smaller fractions of NK cells the lower the starting concentration was (FIG. 1J).

Without being bound by theory, the inventors hypothesise that the reduced NK cell growth at higher cell density could be because there is an increased competition for nutrients or accumulation of inhibitory by-products. To evaluate the turnover of nutrients, glucose and lactate levels were measured for one healthy donor (HD3; FIG. 2A).

High lactate levels were an indication of reduced sub-sequent cell growth (compare lactate day 6 to growth rate day 8) (FIG. 2B).

Surprisingly, CD38 expression was reduced to a lower level when cultures were started at lower seeding densities (FIG. 3).

### Results—Expansions in Bioreactor

The hypothesis that an increased medium exchange could favour the growth of NK cells resulting in higher NK cell percentages in the product at harvest was also supported by observations of our bioreactor expansion cultures, especially when comparing them to the small-scale expansions run in parallel.

Expansions 1 and 2 were a comparison between fresh (1) and frozen (2) starting material from the same donor. Total cell expansion was very similar between the two runs (FIG. 4A). However, the percentage of NK was higher in the expansion from the frozen starting material resulting in a better NK cell yield (FIG. 4B). But neither of the two had an NK cell expansion as the accompanying small-scale culture.

Expansion culture 3 with fresh staring material showed an even higher difference between the bioreactor and the parallel small-scale culture. The small-scale culture contained over 80% NK cells at harvest (FIG. 5B) while the bioreactor culture contained only 19% (FIG. 5C).

The NK cell expansion in the bioreactor slowed down around day 9. The lactate concentration in the bioreactor culture reached levels of over 30 mM by day 7 (FIG. 5D, E). The accumulating lactate and the lower glucose levels indicate that the cells in the bioreactor had a much lower exchange in their culture medium than the cells growing in the flask. Therefore, we considered the cell-specific prefusion rate (CSPR), which is calculated as the volume of fresh medium added per million cells in culture. The difference in the CSPR could be an explanation for the better growth of the cultures in flasks (FIG. 6).

In order to improve the cell growth in expansion culture 4, the perfusion rate was increased (“medium” in Table 1). The total cell expansion was similar to the expansion in the parallel small-scale culture (FIG. 7A). However, the percentage of NK cells was only 25% in the bioreactor culture compared to 57% in the small-scale culture (FIG. 7B, C). The CSPR in the bioreactor was still much lower than in the flask cultures, even though both cultures started with similar rates at day 5 (FIG. 8).

In expansion 5, the perfusion rate was increased further (“high” in Table 1) and the starting cell concentration was reduced to 0.25×10⁶/ml. The total cell expansion was similar between bioreactor and small-scale culture (FIG. 9A). However, importantly the percentage of NK cells at harvest was higher in the bioreactor (FIG. 9B).

Expansions 6 and 7 were performed with the same parameters as expansion 5. In both expansions, the total cell expansion in the bioreactor was lower than in the small-scale cultures (FIG. 10A, B). However, importantly, the percentage of NK cells at harvest day was much higher in the bioreactor (FIG. 10C, F).

The total cell expansion in the three bioreactor cultures with the low starting concentration of 0.25×10⁶ cells/ml and high perfusion rate (expansions 5, 6, and 7) were variable (FIG. 11A). However, importantly the percentage of NK cells in the products of these expansions was higher (FIG. 11B).

Finally, we validated the process with low starting concentration and high perfusion in three expansion cultures with starting material from multiple myeloma (MM) patients (“Val1”, “Val2” and “Val3”) and one with starting material from a healthy donor (“Val4”) (FIG. 12). As in the previous cultures, the expansion of total cells was variable (FIG. 12A). During the third expansion (val3) a technical error led to an interrupted perfusion after day 10 that was fixed on day 13. This disturbed the growth of the cells, but the expansion culture recovered ending with a 90-fold total cell expansion.

Nevertheless, all three MM cultures as well as the healthy donor culture had a high percentage of NK cells at harvest (53%, 93%, 98%, and 65%, respectively; FIG. 12F, G, H, I).

In summary, the six bioreactor expansion cultures with a starting concentration of 0.25×10⁶ cells/mL and a high perfusion rate showed total cell expansions between 26- and 211-fold, with a median of 112.4-fold (Table 2, FIG. 11A, and FIG. 12). Importantly, the percentage of NK cells at harvest was between 31 and 98% with a median of 64.7% (Table 2, FIG. 9B, 10C, F, and FIG. 12F, G, H, I).

### DISCUSSION

Despite high inter-donor variability, expansion cultures with a lower starting concentration of PBMCs are feasible with healthy donor cells, as well as MM patient cells. The total cell expansion of the six experimental bioreactor cultures was lower compared to the previous process, however the median percentage of NK cells was higher. This suggests that NK cell growth is favoured by a higher CSPR.

### Materials and Methods

### Small Scale Cultures

Blood cells from three healthy donors (HD1-HD3), and two MM patients (MM01, MM02) were used in NK cell cultures. PBMCs were purified by ficoll gradient separation of “buffy coats” according to manufacturer's protocol. Specifically, anticoagulant-treated blood is carefully layered on a viscous density gradient media according to manufacturer's instructions (Ficoll-Paque (GE healthcare). Specifically, the lymphocytes are then separated from erythrocytes by density-based gradient centrifugation at 800×g, minimum acceleration without break. Cell layer extracted by pipette and washed 3 times in PBS+0.5% HSA at 300×g. The bottom layer contains sedimented, aggregated erythrocytes, the next layer mostly contains granulocytes, the top layer contains plasma and the intermediate layer, and the interface between the plasma and the Ficoll-Paque layer holds PBMCs mostly made up of lymphocytes. The isolated PBMCs are washed several times with phosphate-buffered saline (PBS) containing 0.5% human serum albumin (HSA) and counted.

Separated PBMCs were used as starting material for NK-cell cultures, either directly or after a freeze-thawing cycle, and this is defined as day 0 of culture.

### Expansion in Cell Culture Flasks

Cells were counted and seeded at different concentrations in a final volume of 2 ml/well (6-well plates, Sarstedt), or 8 ml (T-25 bottles Thermo Scientific) of media (SCGM—(Cellgenix)) with 5% human serum (Access Biologicals) and 500 U/ml IL-2 (Novartis); bioreactor cultures also supplemented with 0.1% Pluronic (Gibco/Thermo Fischer Scientific)

Cells were induced with 10 ng/ml anti-CD3 (Miltenyi Biotech) (freeze-thawed samples were induced after overnight recovery, i.e. on day 1 of culture), and kept in culture the following five days. Fresh IL-2 (500 U/ml) was added Monday-Friday, and all cultures were induced on a Wednesday. After the initial 5-day incubation, cells were cultured for a total of 20-22 days and cells were scraped off the bottom and suspended in the culture media to obtain a homogenous mix every two to three days to allow for counting. Culture volume was measured with the pipette, and a small fraction was taken for cell counting with trypan blue. During medium replenishment, the cell concentration was adjusted to 0.5-1×10⁶ cells/mL in each culture. Importantly, all parallel cultures were split to the same concentration, it was only the concentration at culture start that differed. Total cell number was calculated from cell count and measured volume, and cell expansion was calculated as the fold increase of total cell numbers.

Cells were also taken from the culture for FACS analysis of cell types or NK cell activity by degranulation assay, and for glucose and lactate measurements.

### Expansion in Bioreactor

Cells for bioreactor cultures were prepared as above, with the supplement of 0.1% Pluronic F68.

Cultures were started in a volume of 500 ml in a 2-litre bioreactor bag (Cellbag, 2 L Perf, pH, DO (GE healthcare) on a Xuri W25 bioreactor (Cytiva). (Settings: 37° C., rocking at 6 rpm, 6 degrees angle, 5% CO₂ mixed with compressed air, flow rate (flow of compressed air/5% CO₂) 0.1 l/min).

Initial bioreactor cultures in Xuri W25 were started at 0.5×10⁶ cells/ml and followed the feeding regime corresponding to “low” perfusion in Table 1. A small-scale culture in T25 tissue culture flask was grown in parallel as described above. For cultures from fresh PBMCs 10 ng/ml anti-CD3 was directly added to the cell suspension, for freeze-thawed material anti-CD3 was added the following day. Anti-CD3 was prepared with 500 U/ml IL2 in a syringe, and injected into the bioreactor bag through the sample port.

After the five-day induction with anti-CD3, cells were counted daily on weekdays. The bioreactor bag was shaken to dissolve cell aggregates. A sample was withdrawn from the sample port using a 3 ml or 5 ml syringe attached through the luer connection. A fraction was used for cell count by trypan blue or nucleocounter NC-200 (ChemoMetec) with Via-1 cassettes (ChemoMetec). One sample was used for glucose and lactate measurements and remaining cells were frozen for sub-sequent FACS analysis. Cells were diluted daily, based on cell count to keep a constant concentration until the maximal culture volume of 1 litre for 2-litre bioreactor bag was obtained. Different perfusion steps were started when pre-set criteria in terms of cell count and lactate levels were met (Table 1).

TABLE 1
Criteria for four perfusion steps in the three different strategies
used. For the “high” perfusion strategy there was also an
upper limit of 25 mM lactate in cultures prompting increased perfusion.

| Perfusion step | low | | medium | | high | |
|---|---|---|---|---|---|---|
| Target | 0.5M | cells/ml | 0.5M | cells/ml | 0.5M | cells/ml |
| concentration | | | | | | |
| during initial | | | | | | |
| volume increase | | | | | | |
| 1 - 300 ml/day | >3M | cells/ml | >1M | cells/ml | — | |
| 2 - 500 ml/day | >7M | cells/ml | >3M | cells/ml | >1M | cells/ml |
| 3 - 750 ml/day | >15M | cells/ml | >9M | cells/ml | >3M | cells/ml |
| 4 - 1000 ml/day | >25M | cells/ml | >20M | cells/ml | >9M | cells/ml |

TABLE 3
Results for the “high” perfusion strategy.

[symbol]

| | Start of manufacturing | | | | At harvest | | | | |
|---|---|---|---|---|---|---|---|---|---|
| | Total cell | Total cell | | | Total cell | | Fold | | Fold |
| Expansion | number | concentration | % NK | Culture | number | | expansion | % NK | expansion |
| culture | (10⁶) | (10⁶/ml) | cells | Perfusion | (10⁶) | Day | (total) | cells | NK cells |
| 5 | 125 | 0.25 | 12.4% | High | 26400 | 15 | 211.2 | 30.8% | 524.6 |
| 6 | 125 | 0.25 | 6.4% | High | 3900 | 23 | 31.2 | 69.0% | 334.8 |
| 7 | 125 | 0.25 | 18.2% | High | 15680 | 20 | 125.4 | 44.5% | 306.7 |
| Val1 | 115 | 0.23 | 16.8% | High | 3021 | 24 | 26.3 | 52.7% | 82.4 |
| Val2 | 125 | 0.25 | 11.2% | High | 14050 | 21 | 112.4 | 92.2% | 952.2 |
| Val3 | 125 | 0.25 | 42.5% | High | 11150 | 20 | 89.2 | 97.9% | 205.5 |
| Val4 | 121 | 0.242 | 13.2% | High | 21700 | 17 | 179.6 | 64.7% | 880.5 |

[symbol]

indicates data missing or illegible when filed

### FACS Analysis

The cell phenotype and percentage of subpopulations was analysed by flow cytometry. All antibody staining for flow cytometry were done according to the following protocol cells were washed in PBS, stained with LIVE/DEAD fixable Aqua dead cell stain (Invitrogen), and washed again with PBS, resuspended in reagent diluent concentrate 2 (R & D systems) before incubation with antibodies for 30 min 4° C. aCD45-PerCP (clone 2D1), aCD3-PE (SK7), aCD56-APC (NCAM16.2) and aCD38-PE-Cy7 (HIT2) CD14-V500, CD19-V500, NKp44-BB515, NKp30-BV421, CD16-APC-H7 purchased from BD Biosciences, San Jose, CA, USA). Then washed in PBS and fixated in CellFix (BD). The samples were run on a FACS Verse (BD Biosciences, San Jose, CA, USA) and analysed by FlowJo according to Manufacturer's instructions.

### Glucose/Lactate Measurements

Glucose/lactate measurements were carried out on a Biosen instrument (EKF Diagnostic), according to manufacturer's instructions.

1. A method for expanding a population of natural killer (NK) cells, the method comprising:
i. providing an initial population of cells comprising NK cells;
ii. introducing the initial population of cells into a closed culture system at a concentration of less than 0.5×10⁶ cells/ml of cell culture medium; and
iii. expanding the initial population of cells in the closed culture system, under perfusion conditions effective to produce an expanded population of NK cells, preferably wherein the number of NK cells in the expanded population has increased 50-fold relative to the number of NK cells in the initial population.

2. The method according to claim 1, wherein step (ii) comprises
(a) introducing the initial population of cells into a closed culture system at a concentration of less than 0.5×10⁶ cells/ml of cell culture medium comprising one or more NK cell activating agent; and
(b) culturing the cells at a concentration that does not exceed about 1×10⁵ cells/ml.

3. The method according to claim 1, wherein the method does not comprise feeder cells.

4. The method according to claim 1, wherein the perfusion conditions in step (iii) comprise a perfusion rate effective to maintain lactate levels at 35 mM or less, such as at 30 mM or less.

5. The method according to claim 1, wherein step (iii) is performed until the total number of cells has expanded at least 10-fold relative to the total number of cells in the initial population.

6. The method according to claim 1, wherein the initial population of cells is selected from the group comprising: a population of peripheral blood mononuclear cells (PBMCs), a population of cells derived from cord blood, a population of cells derived from a cell line, a population of cells derived from primary cells, and a population of cells derived from stem cells.

7. The method according to claim 1, wherein steps (ii) and (iii) comprise culturing the cell population in the presence of Interleukin 2 (IL-2).

8. The method according to claim 2, wherein the NK cell activating agent is an anti-CD3 antibody.

9. The method according to claim 2, wherein step (ii) (b) comprises culturing the initial population of cells for about 4-10 days, optionally 5 days.

10. The method according to claim 1, wherein the perfusion conditions comprise:
a perfusion rate of about 0.4-0.6× maximal culture vessel operating volume per 24 hours when the cell population has a concentration of between I×1O⁶ cells/ml and 3×10⁵ cells/ml; and/or
a perfusion rate of about 0.6-0.9× maximal culture vessel operating volume per 24 hours when the cell population has a concentration of between 3×10⁶ cells/ml and 9×10⁶ cells/ml; and/or
a perfusion rate of about 0.9-1.2× maximal culture vessel operating volume per 24 hours after the cell population has reached a concentration of at least 9×10⁶ cells/ml.

11. The method according to claim 1, wherein step (iii) comprises expanding the initial cell population for at least 9 days.

12. The method according to claim 1, further comprising the step, performed after step (iii), of:
(iv) harvesting the expanded cell population.

13. The method according to claim 1, wherein the closed culture system comprises a culture vessel positioned on a platform capable of rocking.

14. The method according to claim 1, wherein the closed culture system is a non-static perfusion bioreactor.

15. The method according to claim 14, wherein the bioreactor is maintained at a rocking rate of 4-8 rocks per minute (rpm), preferably 6 rpm.

16. The method according to claim 15, wherein the bioreactor is maintained at a rocking rate of 4-8 rpm and at an angle between 4-8 degrees, preferably 6 degrees.

17. The method according to claim 1, wherein the NK cells in the expanded population have increased cytotoxicity relative to NK cells in the initial population of cells.

18. The method according to claim 1, wherein the proportion of NK cells expressing surface CD38 in the expanded population is less than 70%.

19. The method according to claim 1, wherein the proportion of NK cells in the expanded population that express surface CD38 is less than the proportion of cells in the initial population that express surface CD38.

20. A population of activated natural killer (NK) cells with the phenotype CD3 CD56⁺ obtainable or obtained by a method according to claim 1.

21. A population of activated natural killer (NK) cells with the phenotype CD3˜CD56⁺, wherein less than 70% of the NK cells express surface CD38, optionally which are obtainable or obtained by a method according to claim 1.

22. A pharmaceutical composition comprising a population of activated NK cells as defined in claim 20, and a pharmaceutically acceptable, diluent, carrier, or excipient.

23. A method of treating a disorder comprising administering a population of activated NK cells, or the pharmaceutical composition as defined in claim 21—to a subject in need thereof.

24. A method of adoptive cell therapy comprising administering a population of activated NK cells, or the pharmaceutical composition as defined in claim 21.

25. A method of manufacturing a medicament for adoptive cell therapy comprising a population of NK cells, or the pharmaceutical composition as defined in claim 22.

26. A method of treating and/or preventing cancer and/or a viral infection comprising administering a population of NK cells, or the pharmaceutical composition as defined in claim 22.

27. A method of manufacturing a medicament for treating and/or preventing cancer and/or a viral infection in a patient comprising administering Use of a population of NK cells, or the pharmaceutical composition as defined in claim 22 to a patient.

28. A method of treating and/or preventing cancer and/or a viral infection in a patient, comprising administering a population of NK cells, or the pharmaceutical composition as defined in claim 22, to the patient.

29. The method of claim 28, wherein the cancer is a haematological cancer, selected from the group consisting of: multiple myeloma, lymphoma, leukaemia and chronic myeloproliferative diseases.

30. A method, a population of NK cells, a population of NK cells for use, use of a population of NK cells, or pharmaceutical composition, substantially as described herein with reference to the accompanying claims and examples.
